SEQUENCE LISTING (115 sequences)

SEQ ID NO: 1 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 2 - protein, 165 aa, Artificial Sequence
  synthetic polypeptide
MEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 3 - protein, 273 aa, Mus musculus
MKKTQTWIITCIYLQLLLFNPLVKTKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVAASSLRNDSSSSNRKAAKAPEDSGLQWTAMALPALISLVIGFAFGALYWKKKQSSLTRAVENIQINEEDNEISMLQQKEREFQEV

SEQ ID NO: 4 - protein, 165 aa, Artificial Sequence
  synthetic polypeptide
MKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVA

SEQ ID NO: 5 - protein, 273 aa, Rattus norvegicus
MKKTQTWIITCIYLQLLLFNPLVKTQEICRNPVTDNVKDITKLVANLPNDYMITLNYVAGMDVLPSHCWLRDMVTHLSVSLTTLLDKFSNISEGLSNYSIIDKLGKIVDDLVACMEENAPKNVKESLKKPETRNFTPEEFFSIFNRSIDAFKDFMVASDTSDCVLSSTLGPEKDSRVSVTKPFMLPPVAASSLRNDSSSSNRKAAKSPEDPGLQWTAMALPALISLVIGFAFGALYWKKKQSSLTRAVENIQINEEDNEISMLQQKEREFQEV

SEQ ID NO: 6 - protein, 165 aa, Artificial Sequence
  synthetic polypeptide
MQEICRNPVTDNVKDITKLVANLPNDYMITLNYVAGMDVLPSHCWLRDMVTHLSVSLTTLLDKFSNISEGLSNYSIIDKLGKIVDDLVACMEENAPKNVKESLKKPETRNFTPEEFFSIFNRSIDAFKDFMVASDTSDCVLSSTLGPEKDSRVSVTKPFMLPPVA

SEQ ID NO: 7 - protein, 11 aa, Artificial Sequence
  synthetic polypeptide
YGRKKRRQRRR

SEQ ID NO: 8 - protein, 9 aa, Artificial Sequence
  synthetic polypeptide
RKKRRQRRR

SEQ ID NO: 9 - protein, 16 aa, Artificial Sequence
  synthetic polypeptide
RQIKIWFQNRRMKWKK

SEQ ID NO: 10 - protein, 19 aa, Artificial Sequence
  synthetic polypeptide
HHHHRKKRRQRRRRHHHHH

SEQ ID NO: 11 - protein, 30 aa, Artificial Sequence
  synthetic polypeptide
MVKSKIGSWILVLFVAMWSDVGLCKKRPKP

SEQ ID NO: 12 - protein, 27 aa, Artificial Sequence
  synthetic polypeptide
GALFLGFLGAAGSTMGAWSQPKKKRKV

SEQ ID NO: 13 - DNA, 6060 bp, Artificial Sequence
  synthetic polynucleotide
aaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgtatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgactgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaagctcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaagaattttattatatgagagggtgaaacctaaaatgaaatgaaatcagacccttgtcttacaccataaacaaaaataaatttgaatgggttaaagaattaaactaagacctaaaaccataaaaatttttaaagaaatcaaaagaagaaaattctaatattcatgttgcagccgttttttgaatttgatatgagaagcaaaggcaacaaaaggaaaaataaagaagtgaggctacatcaaactaaaaaatttccacacaaaaaagaaaacaatgaacaaatgaaaggtgaaccatgaaatggcatatttgcaaaccaaatatttcttaaatattttggttaatatccaaaatatataagaaacacagatgattcaataacaaacaaaaaattaaaaataggaaaataaaaaaattaaaaagaagaaaatcctgccatttatgcgagaattgatgaacctggaggatgtaaaactaagaaaaataagcctgacacaaaaagacaaatactacacaaccttgctcatatgtgaaacataaaaaagtcactctcatggaaacagacagtagaggtatggtttccaggggttgggggtgggagaatcaggaaactattactcaaagggtataaaatttcagttatgtgggatgaataaattctagatatctaatgtacagcatcgtgactgtagttaattgtactgtaagtatatttaaaatttgcaaagagagtagatttttttgtttttttagatggagttttgctcttgttgtccaggctggagtgcaatggcaagatcttggctcactgcaacctccgcctcctgggttcaagcaaatctcctgcctcagcctcccgagtagctgggattacaggcatgcgacaccatgcccagctaattttgtatttttagtagagacggggtttctccatgttggtcaggctgatccgcctcctcggccaccaaagggctgggattacaggcgtgaccaccgggcctggccgagagtagatcttaaaagcatttaccacaagaaaaaggtaactatgtgagataatgggtatgttaattagcttgattgtggtaatcatttcacaaggtatacatatattaaaacatcatgttgtacaccttaaatatatacaatttttatttgtgaatgatacctcaataaagttgaagaataataaaaaagaatagacatcacatgaattaaaaaactaaaaaataaaaaaatgcatcttgatgattagaattgcattcttgatttttcagatacaaatatccatttgactg

SEQ ID NO: 14 - DNA, 850 bp, Artificial Sequence
  synthetic polynucleotide
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 15 - DNA, 12 bp, Artificial Sequence
  synthetic polynucleotide
gaaagaaagaga

SEQ ID NO: 16 - DNA, 18 bp, Artificial Sequence
  synthetic polynucleotide
tgccctgaaagaaagaga

SEQ ID NO: 17 - DNA, 17 bp, Artificial Sequence
  synthetic polynucleotide
agaatggtgcaaagagg

SEQ ID NO: 18 - DNA, 18 bp, Artificial Sequence
  synthetic polynucleotide
acatgattagcaaaaggg

SEQ ID NO: 19 - DNA, 12 bp, Artificial Sequence
  synthetic polynucleotide
ctttctttctct

SEQ ID NO: 20 - DNA, 12 bp, Artificial Sequence
  synthetic polynucleotide
tctctttctttc

SEQ ID NO: 21 - DNA, 18 bp, Artificial Sequence
  synthetic polynucleotide
tctctttctttcagggca

SEQ ID NO: 22 - DNA, 18 bp, Artificial Sequence
  synthetic polynucleotide
cccttttgctaatcatgt

SEQ ID NO: 23 - DNA, 17 bp, Artificial Sequence
  synthetic polynucleotide
cctctttgcaccattct

SEQ ID NO: 24 - DNA, 12 bp, Artificial Sequence
  synthetic polynucleotide
  misc_feature (1)..(1): n = Pseudoisocytosine
  misc_feature (5)..(5): n = Pseudoisocytosine
  misc_feature (9)..(9): n = Pseudoisocytosine
  misc_feature (11)..(11): n = Pseudoisocytosine
ntttntttntnt

SEQ ID NO: 25 - DNA, 10 bp, Artificial Sequence
  synthetic polynucleotide
  misc_feature (2)..(2): n = Pseudoisocytosine
  misc_feature (7)..(7): n = Pseudoisocytosine
  misc_feature (10)..(10): n = Pseudoisocytosine
tnttttnttn

SEQ ID NO: 26 - DNA, 10 bp, Artificial Sequence
  synthetic polynucleotide
cttcttttct

SEQ ID NO: 27 - DNA, 10 bp, Artificial Sequence
  synthetic polynucleotide
  misc_feature (3)..(3): n = Pseudoisocytosine
  misc_feature (6)..(6): n = Pseudoisocytosine
  misc_feature (10)..(10): n = Pseudoisocytosine
ttnttntttn

SEQ ID NO: 28 - DNA, 10 bp, Artificial Sequence
  synthetic polynucleotide
ctttcttctt

SEQ ID NO: 29 - DNA, 10 bp, Artificial Sequence
  synthetic polynucleotide
  misc_feature (1)..(1): n = Pseudoisocytosine
  misc_feature (2)..(2): n = Pseudoisocytosine
  misc_feature (3)..(3): n = Pseudoisocytosine
  misc_feature (5)..(5): n = Pseudoisocytosine
  misc_feature (6)..(6): n = Pseudoisocytosine
  misc_feature (9)..(9): n = Pseudoisocytosine
nnntnnttnt

SEQ ID NO: 30 - DNA, 10 bp, Artificial Sequence
  synthetic polynucleotide
tcttcctccc

SEQ ID NO: 31 - DNA, 15 bp, Artificial Sequence
  synthetic polynucleotide
cttctccacaggagt

SEQ ID NO: 32 - DNA, 18 bp, Artificial Sequence
  synthetic polynucleotide
cttctccacaggagtcag

SEQ ID NO: 33 - DNA, 22 bp, Artificial Sequence
  synthetic polynucleotide
cttctccacaggagtcaggtgc

SEQ ID NO: 34 - DNA, 15 bp, Artificial Sequence
  synthetic polynucleotide
tctccttaaacctgt

SEQ ID NO: 35 - DNA, 18 bp, Artificial Sequence
  synthetic polynucleotide
tctccttaaacctgtctt

SEQ ID NO: 36 - DNA, 16 bp, Artificial Sequence
  synthetic polynucleotide
tcttctctgtctccac

SEQ ID NO: 37 - DNA, 18 bp, Artificial Sequence
  synthetic polynucleotide
tcttctctgtctccacat

SEQ ID NO: 38 - DNA, 17 bp, Artificial Sequence
  synthetic polynucleotide
tttcctctatgggtaag

SEQ ID NO: 39 - DNA, 17 bp, Artificial Sequence
  synthetic polynucleotide
cttacccatagaggaaa

SEQ ID NO: 40 - DNA, 17 bp, Artificial Sequence
  synthetic polynucleotide
atgccaactagaagagg

SEQ ID NO: 41 - DNA, 17 bp, Artificial Sequence
  synthetic polynucleotide
cctcttctagttggcat

SEQ ID NO: 42 - DNA, 18 bp, Artificial Sequence
  synthetic polynucleotide
ctttcccttgtatctttt

SEQ ID NO: 43 - DNA, 18 bp, Artificial Sequence
  synthetic polynucleotide
aaaagatacaagggaaag

SEQ ID NO: 44 - DNA, 10 bp, Artificial Sequence
  synthetic polynucleotide
gaaggagaaa

SEQ ID NO: 45 - DNA, 10 bp, Artificial Sequence
  synthetic polynucleotide
agaaaaaagg

SEQ ID NO: 46 - DNA, 17 bp, Artificial Sequence
  synthetic polynucleotide
tttcctctatgggtaag

SEQ ID NO: 47 - DNA, 10 bp, Artificial Sequence
  synthetic polynucleotide
cttcctcttt

SEQ ID NO: 48 - DNA, 10 bp, Artificial Sequence
  synthetic polynucleotide
tttctccttc

SEQ ID NO: 49 - DNA, 18 bp, Artificial Sequence
  synthetic polynucleotide
tttctccttcagtgttca

SEQ ID NO: 50 - DNA, 20 bp, Artificial Sequence
  synthetic polynucleotide
tccttttgctcacctgtggt

SEQ ID NO: 51 - DNA, 10 bp, Artificial Sequence
  synthetic polynucleotide
tcttttttcc

SEQ ID NO: 52 - DNA, 10 bp, Artificial Sequence
  synthetic polynucleotide
ccttttttct

SEQ ID NO: 53 - DNA, 18 bp, Artificial Sequence
  synthetic polynucleotide
ccttttttctggctaagt

SEQ ID NO: 54 - DNA, 10 bp, Artificial Sequence
  synthetic polynucleotide
  misc_feature (1)..(1): n = Pseudoisocytosine
  misc_feature (4)..(4): n = Pseudoisocytosine
  misc_feature (5)..(5): n = Pseudoisocytosine
  misc_feature (7)..(7): n = Pseudoisocytosine
nttnntnttt

SEQ ID NO: 55 - DNA, 18 bp, Artificial Sequence
  synthetic polynucleotide
tttttctgtaatttttaa

SEQ ID NO: 56 - DNA, 17 bp, Artificial Sequence
  synthetic polynucleotide
tctttctctgcaaactt

SEQ ID NO: 57 - DNA, 18 bp, Artificial Sequence
  synthetic polynucleotide
tttctttaagaacgagca

SEQ ID NO: 58 - DNA, 10 bp, Artificial Sequence
  synthetic polynucleotide
ctcttcttct

SEQ ID NO: 59 - DNA, 10 bp, Artificial Sequence
  synthetic polynucleotide
tcttcttctc

SEQ ID NO: 60 - DNA, 15 bp, Artificial Sequence
  synthetic polynucleotide
tcttcttctcatttc

SEQ ID NO: 61 - DNA, 12 bp, Artificial Sequence
  SEQ ID NO61
ctgctcggaaga

SEQ ID NO: 62 - DNA, 12 bp, Artificial Sequence
  synthetic polynucleotide
tcttccgagcag

SEQ ID NO: 63 - DNA, 15 bp, Artificial Sequence
  synthetic polynucleotide
ccttcaccaagggga

SEQ ID NO: 64 - DNA, 15 bp, Artificial Sequence
  synthetic polynucleotide
tccccttggtgaagg

SEQ ID NO: 65 - DNA, 22 bp, Artificial Sequence
  synthetic polynucleotide
tctgggttaaggtaatagcaat

SEQ ID NO: 66 - DNA, 30 bp, Artificial Sequence
  synthetic polynucleotide
  misc_feature (1)..(1): n = Pseudoisocytosine
  misc_feature (1)..(1): Linked to lys-lys-lys
  misc_feature (5)..(5): n = Pseudoisocytosine
  misc_feature (9)..(9): n = Pseudoisocytosine
  misc_feature (11)..(11): n = Pseudoisocytosine
  misc_feature (12)..(13): Linked by three 8-amino-2, 6, 10-trioxaoctanoic acid, three 8-amino-3,6-dioxaoctanoic acid, or three 6-aminohexanoic acid molecules
  misc_feature (30)..(30): Linked to lys-lys-lys
ntttntttntnttctctttctttcagggca

SEQ ID NO: 67 - DNA, 25 bp, Artificial Sequence
  synthetic polynucleotide
  misc_feature (1)..(1): Linked to lys-lys-lys
  misc_feature (5)..(5): n = Pseudoisocytosine
  misc_feature (6)..(6): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttttnnncccttttgctaatcatgt

SEQ ID NO: 68 - DNA, 24 bp, Artificial Sequence
  synthetic polynucleotide
  misc_feature (1)..(1): Linked to lys-lys-lys
  misc_feature (4)..(4): n = Pseudoisocytosine
  misc_feature (6)..(6): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tttntnncctctttgcaccattct

SEQ ID NO: 69 - DNA, 20 bp, Artificial Sequence
  synthetic polynucleotide
  misc_feature (1)..(1): Linked to lys-lys-lys
  misc_feature (2)..(2): n = Pseudoisocytosine
  misc_feature (7)..(7): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
tnttttnttncttcttttct

SEQ ID NO: 70 - DNA, 20 bp, Artificial Sequence
  synthetic polynucleotide
  misc_feature (1)..(1): Linked to lys-lys-lys
  misc_feature (3)..(3): n = Pseudoisocytosine
  misc_feature (6)..(6): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
ttnttntttnctttcttctt

SEQ ID NO: 71 - DNA, 20 bp, Artificial Sequence
  synthetic polynucleotide
  misc_feature (1)..(1): Linked to lys-lys-lys
  misc_feature (1)..(1): n = Pseudoisocytosine
  misc_feature (2)..(2): n = Pseudoisocytosine
  misc_feature (3)..(3): n = Pseudoisocytosine
  misc_feature (5)..(5): n = Pseudoisocytosine
  misc_feature (6)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
nnntnnttnttcttcctccc

SEQ ID NO: 72 - DNA, 22 bp, Artificial Sequence
  synthetic polynucleotide
  misc_feature (1)..(1): Linked to lys-lys-lys
  misc_feature (1)..(1): n = Pseudoisocytosine
  misc_feature (2)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
nntnttncttctccacaggagt

SEQ ID NO: 73 - DNA, 22 bp, Artificial Sequence
  synthetic polynucleotide
  misc_feature (1)..(1): Linked to lys-lys-lys
  misc_feature (3)..(3): n = Pseudoisocytosine
  misc_feature (4)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
ttnntnttctccttaaacctgt

SEQ ID NO: 74 - DNA, 25 bp, Artificial Sequence
  synthetic polynucleotide
  misc_feature (1)..(1): Linked to lys-lys-lys
  misc_feature (3)..(3): n = Pseudoisocytosine
  misc_feature (4)..(4): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ttnntnttctccttaaacctgtctt

SEQ ID NO: 75 - DNA, 24 bp, Artificial Sequence
  synthetic polynucleotide
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (24)..(24): Linked to lys-lys-lys
tntnttnttcttctctgtctccac

SEQ ID NO: 76 - DNA, 25 bp, Artificial Sequence
  synthetic polynucleotide
  misc_feature (1)..(1): Linked to lys-lys-lys
  misc_feature (1)..(1): n = Pseudoisocytosine
  misc_feature (2)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
nntnttncttctccacaggagtcag

SEQ ID NO: 77 - DNA, 29 bp, Artificial Sequence
  synthetic polynucleotide
  misc_feature (1)..(1): Linked to lys-lys-lys
  misc_feature (1)..(1): n = Pseudoisocytosine
  misc_feature (2)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (29)..(29): Linked to lys-lys-lys
nntnttncttctccacaggagtcaggtgc

SEQ ID NO: 78 - DNA, 26 bp, Artificial Sequence
  synthetic polynucleotide
  misc_feature (1)..(1): Linked to lys-lys-lys
  misc_feature (2)..(2): n = Pseudoisocytosine
  misc_feature (4)..(4): n = Pseudoisocytosine
  misc_feature (7)..(7): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntnttnttcttctctgtctccacat

SEQ ID NO: 79 - DNA, 25 bp, Artificial Sequence
  synthetic polynucleotide
  misc_feature (1)..(1): Linked to lys-lys-lys
  misc_feature (2)..(2): n = Pseudoisocytosine
  misc_feature (4)..(4): n = Pseudoisocytosine
  misc_feature (5)..(5): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tntnnttttttcctctatgggtaag

SEQ ID NO: 80 - DNA, 25 bp, Artificial Sequence
  synthetic polynucleotide
  misc_feature (1)..(1): Linked to lys-lys-lys
  misc_feature (2)..(2): n = Pseudoisocytosine
  misc_feature (5)..(5): n = Pseudoisocytosine
  misc_feature (7)..(7): n = Pseudoisocytosine
  misc_feature (8)..(8): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttntnncctcttctagttggcat

SEQ ID NO: 81 - DNA, 27 bp, Artificial Sequence
  synthetic polynucleotide
  misc_feature (1)..(1): Linked to lys-lys-lys
  misc_feature (3)..(3): n = Pseudoisocytosine
  misc_feature (4)..(4): n = Pseudoisocytosine
  misc_feature (5)..(5): n = Pseudoisocytosine
  misc_feature (9)..(9): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttnnntttnctttcccttgtatctttt

SEQ ID NO: 82 - DNA, 28 bp, Artificial Sequence
  synthetic polynucleotide
  misc_feature (1)..(1): Linked to lys-lys-lys
  misc_feature (1)..(1): n = Pseudoisocytosine
  misc_feature (4)..(4): n = Pseudoisocytosine
  misc_feature (5)..(5): n = Pseudoisocytosine
  misc_feature (7)..(7): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (28)..(28): Linked to lys-lys-lys
nttnntnttttttctccttcagtgttca

SEQ ID NO: 83 - DNA, 27 bp, Artificial Sequence
  synthetic polynucleotide
  misc_feature (1)..(1): Linked to lys-lys-lys
  misc_feature (5)..(5): n = Pseudoisocytosine
  misc_feature (6)..(6): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
ttttnnttccttttgctcacctgtggt

SEQ ID NO: 84 - DNA, 28 bp, Artificial Sequence
  synthetic polynucleotide
  misc_feature (1)..(1): Linked to lys-lys-lys
  misc_feature (2)..(2): n = Pseudoisocytosine
  misc_feature (9)..(9): n = Pseudoisocytosine
  misc_feature (10)..(10): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (28)..(28): Linked to lys-lys-lys
tnttttttnnccttttttctggctaagt

SEQ ID NO: 85 - DNA, 25 bp, Artificial Sequence
  synthetic polynucleotide
  misc_feature (1)..(1): Linked to lys-lys-lys
  misc_feature (2)..(2): Linked to lys-lys-lys
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tnttttttttttctgtaatttttaa

SEQ ID NO: 86 - DNA, 26 bp, Artificial Sequence
  synthetic polynucleotide
  misc_feature (1)..(1): Linked to lys-lys-lys
  misc_feature (2)..(2): n = Pseudoisocytosine
  misc_feature (4)..(4): n = Pseudoisocytosine
  misc_feature (8)..(8): n = Pseudoisocytosine
  misc_feature (9)..(10): Linked by three 8-amino-2, 6, 10-trioxaoctanoic acid, three 8-amino-3,6-dioxaoctanoic acid, or three 6-aminohexanoic acid molecules
  misc_feature (26)..(26): Linked to lys-lys-lys
tntntttnttctttctctgcaaactt

SEQ ID NO: 87 - DNA, 25 bp, Artificial Sequence
  synthetic polynucleotide
  misc_feature (1)..(1): Linked to lys-lys-lys
  misc_feature (4)..(4): n = Pseudoisocytosine
  misc_feature (7)..(8): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
tttnttttttctttaagaacgagca

SEQ ID NO: 88 - DNA, 27 bp, Artificial Sequence
  synthetic polynucleotide
  misc_feature (1)..(1): Linked to lys-lys-lys
  misc_feature (1)..(1): n = Pseudoisocytosine
  misc_feature (6)..(6): n = Pseudoisocytosine
  misc_feature (7)..(7): n = Pseudoisocytosine
  misc_feature (8)..(8): n = Pseudoisocytosine
  misc_feature (8)..(9): Linked by three 8-amino-2, 6, 10-trioxaoctanoic acid, three 8-amino-3,6-dioxaoctanoic acid, or three 6-aminohexanoic acid molecules
  misc_feature (27)..(27): Linked to lys-lys-lys
nttttnnncccttttcaaggtgagtag

SEQ ID NO: 89 - DNA, 25 bp, Artificial Sequence
  synthetic polynucleotide
  misc_feature (1)..(1): Linked to lys-lys-lys
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
  misc_feature (10)..(11): Linked by three 8-amino-2, 6, 10-trioxaoctanoic acid, three 8-amino-3,6-dioxaoctanoic acid, or three 6-aminohexanoic acid molecules
  misc_feature (25)..(25): Linked to lys-lys-lys
ntnttnttnttcttcttctcatttc

SEQ ID NO: 90 - DNA, 20 bp, Artificial Sequence
  synthetic polynucleotide
  misc_feature (1)..(1): Linked to lys-lys-lys
  misc_feature (1)..(1): n = Pseudoisocytosine
  misc_feature (4)..(4): n = Pseudoisocytosine
  misc_feature (5)..(6): Linked by three 8-amino-2, 6, 10-trioxaoctanoic acid, three 8-amino-3,6-dioxaoctanoic acid, or three 6-aminohexanoic acid molecules
  misc_feature (20)..(20): Linked to lys-lys-lys
nttnttcttcttctcatttc

SEQ ID NO: 91 - DNA, 22 bp, Artificial Sequence
  synthetic polynucleotide
  misc_feature (1)..(1): Linked to lys-lys-lys
  misc_feature (3)..(3): n = Pseudoisocytosine
  misc_feature (4)..(4): n = Pseudoisocytosine
  misc_feature (5)..(5): n = Pseudoisocytosine
  misc_feature (6)..(6): n = Pseudoisocytosine
  misc_feature (6)..(7): Linked by three 8-amino-2, 6, 10-trioxaoctanoic acid, three 8-amino-3,6-dioxaoctanoic acid, or three 6-aminohexanoic acid molecules
  misc_feature (22)..(22): Linked to lys-lys-lys
ttnnnnttccccttggtgaagg

SEQ ID NO: 92 - DNA, 18 bp, Artificial Sequence
  synthetic polynucleotide
  misc_feature (1)..(1): Linked to lys-lys-lys
  misc_feature (1)..(1): n = Pseudoisocytosine
  misc_feature (2)..(2): n = Pseudoisocytosine
  misc_feature (5)..(5): n = Pseudoisocytosine
  misc_feature (6)..(7): Linked by three 8-amino-2, 6, 10-trioxaoctanoic acid, three 8-amino-3,6-dioxaoctanoic acid, or three 6-aminohexanoic acid molecules
  misc_feature (18)..(18): Linked to lys-lys-lys
nnttnttcttccgagcag

SEQ ID NO: 93 - DNA, 60 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (57)..(58): Optional phosphorothiate internucleoside linkage
  misc_feature (58)..(59): Optional phosphorothiate internucleoside linkage
  misc_feature (59)..(60): Optional phosphorothiate internucleoside linkage
aaagaataacagtgataatttctgggttaaggcaatagcaatatctctgcatataaatat

SEQ ID NO: 94 - DNA, 70 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (67)..(68): Optional phosphorothiate internucleoside linkage
  misc_feature (68)..(69): Optional phosphorothiate internucleoside linkage
  misc_feature (69)..(70): Optional phosphorothiate internucleoside linkage
cttgccccacagggcagtaacggcagatttttcttccggcgttaaatgcaccatggtgtctgtttgaggt

SEQ ID NO: 95 - DNA, 51 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (48)..(49): Optional phosphorothiate internucleoside linkage
  misc_feature (49)..(50): Optional phosphorothiate internucleoside linkage
  misc_feature (50)..(51): Optional phosphorothiate internucleoside linkage
acagacaccatggtgcacctgactcctgaggagaagtctgccgttactgcc

SEQ ID NO: 96 - DNA, 65 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (62)..(63): Optional phosphorothiate internucleoside linkage
  misc_feature (63)..(64): Optional phosphorothiate internucleoside linkage
  misc_feature (64)..(65): Optional phosphorothiate internucleoside linkage
ttgccccacagggcagtaacggcagacttctcctcaggagtcaggtgcaccatggtgtctgtttg

SEQ ID NO: 97 - DNA, 61 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (58)..(59): Optional phosphorothiate internucleoside linkage
  misc_feature (59)..(60): Optional phosphorothiate internucleoside linkage
  misc_feature (60)..(61): Optional phosphorothiate internucleoside linkage
ttctgtatctatattcatcataggaaacaccaaagataatgttctccttaatggtgccagg

SEQ ID NO: 98 - DNA, 61 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (58)..(59): Optional phosphorothiate internucleoside linkage
  misc_feature (59)..(60): Optional phosphorothiate internucleoside linkage
  misc_feature (60)..(61): Optional phosphorothiate internucleoside linkage
tcttgggattcaataaccttgcagacagtggaggaaggcctttggcgtgataccacaggtg

SEQ ID NO: 99 - DNA, 63 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (60)..(61): Optional phosphorothiate internucleoside linkage
  misc_feature (61)..(62): Optional phosphorothiate internucleoside linkage
  misc_feature (62)..(63): Optional phosphorothiate internucleoside linkage
tccaagtttgcagagaaagataatatagtccttggagaaggaggaatcaccctgagtggaggt

SEQ ID NO: 100 - DNA, 65 bp, Artificial Sequence; identical to SEQ ID NO: 96
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (62)..(63): Optional phosphorothiate internucleoside linkage
  misc_feature (63)..(64): Optional phosphorothiate internucleoside linkage
  misc_feature (64)..(65): Optional phosphorothiate internucleoside linkage

SEQ ID NO: 101 - DNA, 61 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (58)..(59): Optional phosphorothiate internucleoside linkage
  misc_feature (59)..(60): Optional phosphorothiate internucleoside linkage
  misc_feature (60)..(61): Optional phosphorothiate internucleoside linkage
tcttatatctgtactcatcataggaaacaccaaagataatgttctccttgatagtacccgg

SEQ ID NO: 102 - DNA, 60 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (57)..(58): Optional phosphorothiate internucleoside linkage
  misc_feature (58)..(59): Optional phosphorothiate internucleoside linkage
  misc_feature (59)..(60): Optional phosphorothiate internucleoside linkage
attcccgagtagcagatgaccatgacagcttagggcaggaccagccccaagatgactatc

SEQ ID NO: 103 - DNA, 60 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (57)..(58): Optional phosphorothiate internucleoside linkage
  misc_feature (58)..(59): Optional phosphorothiate internucleoside linkage
  misc_feature (59)..(60): Optional phosphorothiate internucleoside linkage
tttaggattcccgagtagcagatgacccctcagagcagcggcaggaccagccccaagatg

SEQ ID NO: 104 - DNA, 65 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (62)..(63): Optional phosphorothiate internucleoside linkage
  misc_feature (63)..(64): Optional phosphorothiate internucleoside linkage
  misc_feature (64)..(65): Optional phosphorothiate internucleoside linkage
gatgactatctttaatgtctggaaattcttccagaattaattaagactgtatggaaaatgagagc

SEQ ID NO: 105 - DNA, 66 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (63)..(64): Optional phosphorothiate internucleoside linkage
  misc_feature (64)..(65): Optional phosphorothiate internucleoside linkage
  misc_feature (65)..(66): Optional phosphorothiate internucleoside linkage
ccccaagatgactatctttaatgtctggaacgatcatcagaattgatactgactgtatggaaaatg

SEQ ID NO: 106 - DNA, 65 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (62)..(63): Optional phosphorothiate internucleoside linkage
  misc_feature (63)..(64): Optional phosphorothiate internucleoside linkage
  misc_feature (64)..(65): Optional phosphorothiate internucleoside linkage
gatgactatctttaatgtctggaaattctactagaattgatactgactgtatggaaaatgagagc

SEQ ID NO: 107 - DNA, 64 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (61)..(62): Optional phosphorothiate internucleoside linkage
  misc_feature (62)..(63): Optional phosphorothiate internucleoside linkage
  misc_feature (63)..(64): Optional phosphorothiate internucleoside linkage
aggacggtcccggcctgcgacacttccgcccataattgttcttcatctgcggggcggggggggg

SEQ ID NO: 108 - DNA, 67 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (64)..(65): Optional phosphorothiate internucleoside linkage
  misc_feature (65)..(66): Optional phosphorothiate internucleoside linkage
  misc_feature (66)..(67): Optional phosphorothiate internucleoside linkage
gggacggcgcccacataggccaaattcaattgctgatcccagcttaagacgtactggtcagcctggc

SEQ ID NO: 109 - DNA, 10 bp, Artificial Sequence
  synthetic polynucleotide
  misc_feature (2)..(2): n = Pseudoisocytosine
  misc_feature (9)..(9): n = Pseudoisocytosine
  misc_feature (10)..(10): n = Pseudoisocytosine
tnttttttnn

SEQ ID NO: 110 - DNA, 10 bp, Artificial Sequence
  synthetic polynucleotide
  misc_feature (1)..(1): n = Pseudoisocytosine
  misc_feature (3)..(3): n = Pseudoisocytosine
  misc_feature (6)..(6): n = Pseudoisocytosine
  misc_feature (9)..(9): n = Pseudoisocytosine
ntnttnttnt

SEQ ID NO: 111 - DNA, 51 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (48)..(49): Optional phosphorothiate internucleoside linkage
  misc_feature (49)..(50): Optional phosphorothiate internucleoside linkage
  misc_feature (50)..(51): Optional phosphorothiate internucleoside linkage
gttcagcgtgtccggcgagggcgaggtgagtctatgggacccttgatgttt

SEQ ID NO: 112 - DNA, 51 bp, Artificial Sequence
  synthetic polynucleotide
  misc_feature (1)..(2): Optional phosphorothiate internucleoside linkage
  misc_feature (2)..(3): Optional phosphorothiate internucleoside linkage
  misc_feature (3)..(4): Optional phosphorothiate internucleoside linkage
  misc_feature (48)..(49): Optional phosphorothiate internucleoside linkage
  misc_feature (49)..(50): Optional phosphorothiate internucleoside linkage
  misc_feature (50)..(51): Optional phosphorothiate internucleoside linkage
aaacatcaagggtcccatagactcacctcgccctcgccggacacgctgaac

SEQ ID NO: 113 - DNA, 23 bp, Artificial Sequence
  synthetic polynucleotide
accattctaaagaataacagtga

SEQ ID NO: 114 - DNA, 22 bp, Artificial Sequence
  synthetic polynucleotide
cctcttacatcagttacaattt

SEQ ID NO: 115 - DNA, 19 bp, Artificial Sequence
  synthetic polynucleotide
tgggttaaggcaatagcaa

(19) United States
(12) Patent Application Publication
(10) Pub. No.: US 2021/0189431 A1
(43) Pub. Date: Jun. 24, 2021

(54) COMPOSITIONS AND METHODS FOR EMBRYONIC GENE EDITING IN VITRO

(71) Applicant: Yale University, New Haven, CT (US)

(72) Inventors: Rachael Putman, Belton, MO (US); Adele S. Ricciardi, New Haven, CT (US); Peter M. Glazer, Guilford, CT (US); W. Mark Saltzman, New Haven, CT (US)

(21) Appl. No.: 17/267,755

(22) PCT Filed: Aug. 12, 2019
(86) PCT No.: PCT/US2019/046163

§ 371 Date: Feb. 10, 2021

Related U.S. Application Data
(60) Provisional application No. 62/717,439, filed on Aug. 10, 2018.

(51) Int. Cl. C12N 15/873; C12N 15/90; C12N 15/88

(52) U.S. Cl. CPC C12N 15/873; C12N 15/88; C12N 15/907

(57) ABSTRACT

Methods for gene editing of embryos in vitro are provided. The methods typically include contacting an embryo in vitro with an effective amount of non-enzymatic (e.g., non-nuclease) gene editing active agent(s) optionally encapsulated, entrapped, complexed to or dispersed in polymeric particles to induce at least one alteration in the genome of the embryo. The embryo can be a single cell zygote, however, treatment of male and female gametes prior to fertilization, and embryos having 2, 4, 8, or 16 cells, and including not only zygotes, but also morulas and blastocysts are also provided. Typically, the embryo is contacted with the particles on culture days 0-6 during or following in vitro fertilization.

### CROSS-REFERENCE TO RELATED APPLICATIONS

This application claims the benefit of and priority to U.S. Ser. No. 62/717,439, filed Aug. 10, 2018, which is specifically incorporated by reference herein in its entirety.

### STATEMENT REGARDING FEDERALLY SPONSORED RESEARCH

This invention was made with government support under Grant Nos. F30HL134252 and 5R01HL125892 awarded by National Institutes of Health. The government has certain rights in the invention.

### REFERENCE TO THE SEQUENCE LISTING

The Sequence Listing submitted as a text file named “YU_7512_PCT” created on Aug. 12, 2019, and having a size of 73,998 bytes is hereby incorporated by reference pursuant to 37 C.F.R. § 1.52(e)(5).

### FIELD OF THE INVENTION

The field of the invention is generally related to compositions and methods for non-enzymatic gene editing of embryos in vitro.

### BACKGROUND OF THE INVENTION

Genome editing has the potential to treat numerous genetic disorders. Embryonic gene editing may be advantageous due to a small number of cells that are rapidly dividing to form the rest of the organism. Changes made in these stem cells may be heritable and can be passed onto daughter cells that will go on to form the rest of the developing organism, potentially allowing for high editing rates that may be maintained throughout adulthood.

Embryonic gene editing could be used for many applications, such as: the creation of genetic mouse models, improving the output of livestock products, and may have clinical significance during in vitro fertilization (IVF) for the treatment or prevention of human disease.

Millions of children every year are born with genetic disorders (Ricciardi et al., Nat Commun, 9(1): p. 2481 (2018)). Embryonic editing during in vitro fertilization (IVF), with over eight million children born worldwide since its introduction in 1978, could present an opportune time to treat these genetic disorders.

Preimplantation genetic diagnosis (PGD), in which disease-causing genetic mutations can be detected from a single cell removed from an 8-cell stage embryo (Kalma et al., Hum Reprod, 33(1): p. 32-38 (2018)), a few cells removed from the trophectoderm of a developing blastocyst (McArthur et al., Prenat Diagn, 28(5): p. 434-42 (2008)), or from gDNA extracted from cell-free growth media (Shamonki et al., Fertil Steril, 106(6): p. 1312-1318 (2016)), is becoming more commonplace. However, the chance for diagnostic error still exists, mainly due to naturally occurring embryo mosaicism, and PGD cannot test for all inherited genetic disorders (Zeng et al., Mol Ther, 26(11): p. 2631-2637 (2018)). Additionally, due to higher numbers of affected embryos in X-linked and autosomal dominant genetic disorders, families may wish for alternative treatments that could allow for higher numbers of embryos to be transferred to improve the chance of the birth of a healthy child. In addition to couples that are known mutation carriers, this approach may also be particularly applicable to parents undergoing IVF who are both homozygous for a monogenic disease. Embryonic gene editing may also have applications in the prevention of diseases such as Alzheimer's disease, by modifying the ApoE4 risk allele, or in HIV, by editing the CCR5 receptor.

Traditional embryonic gene editing approaches involve pronuclear microinjection or electroporation of engineered nucleases, such as CRISPR/Cas9. These procedures are labor intensive and can result in significant embryo loss (Behringer, New York: Cold Spring Harbor Laboratory Press. xxii, 814 pages).

Thus, it is an object of the invention to provide compositions and methods of in vitro embryonic gene editing.

### SUMMARY OF THE INVENTION

Methods for gene editing of embryos in vitro are provided. The methods typically include contacting an embryo in vitro with an effective amount of non-enzymatic (e.g., non-nuclease) gene editing active agent(s) encapsulated, entrapped, complexed to or dispersed in polymeric particles to induce at least one alteration in the genome of the embryo. In some embodiments, particles are not utilitzed, and thus, the methods can include direclty contacting an embryo in vitro with an effective amount of non-enzymatic (e.g., non-nuclease) gene editing active agent(s) to induce at least one alteration in the genome of the embryo.

Most preferably the embryo is a single cell zygote, however, treatment of male and female gametes prior to and during fertilization, and embryos having 2, 4, 8, or 16 cells and including not only zygotes, but also morulas and blastocytes, are also provided. Typically, the embryo is contacted with the particles on culture days 0-6 during or following in vitro fertilization.

The contacting can be adding the particles, or gene editing active agent(s) or a composition including the particles or active agents, to liquid media bathing the embryo. For example, particles or active agents can be pipetted directly into the embryo culture media, whereupon they are taken up by the embryo.

In some embodiments, nanoparticles are utilized for delivery of the gene editing active agents. Nanoparticles are capable of passing through the zona pellucida, a protective glycoprotein coat surrounding the embryo. The particles then degrade, releasing their active agent cargo within the cells whereupon the active agent can achieve site-specific gene editing. This method of delivery is an improvement over more complicated microinjection techniques.

The genomic alteration can be any desirable genomic change. In some embodiments, the alternation is correction of a mutation associated with a genetic disease or disorder. The genetic disease or disorder can be, for example, a monogenic disease or disorder. Illustrative diseases or disorders include, but are not limited to, hemophilias, hemoglobinopathies, cystic fibrosis, xeroderma pigmentosum, lysosomal storage diseases, Huntington's disease, Duchenne muscular dystrophy, and Alzheimer's disease.

Non-enzymatic gene editing active agents are discussed in more detail below. Most typically, the non-enzymatic gene editing active agent(s) is triplex-forming molecules, donor oligonucleotides, or a combination thereof. For example, the gene editing active agent(s) can include a peptide nucleic acid (PNA) tail clamp that enhances recombination of a donor oligonucleotide in the embryonic genome relative to the donor oligonucleotide alone. The active agent(s) can further include the donor oligonucleotide. The PNA tail clamp can include at least one PNA oligomer. At least one of the PNA oligomers of the PNA tail clamp is a modified PNA oligomer having at least one modification at a gamma position of a backbone carbon. The modified PNA oligomer can include, for example, at least one miniPEG modification at a gamma position of a backbone carbon.

Exemplary polymers, particles formed therefrom, and methods of making particles are discussed in more detail below. In some embodiments, the particles are formed of a polymer, copolymer or polymer blend selected from the group consisting of poly(lactic acid) (PLA), poly(glycolic acid) (PGA), poly(lactide-co-glycolide) (PLGA), polyesters, polyanhydrides, poly(ortho)esters, poly(butic acid), poly(valeric acid), poly(caprolactone), poly(hydroxyalkanoates), poly(lactide-co-caprolactone), poly(beta-amino) esters (PBAEs) and poly(amine-co-ester) polymers (PACE). In particular embodiments, the particles are nanoparticles including PLGA.

Embryonic gene editing may be advantageous over adult and human gene editing due to the small number of cells that are rapidly dividing to form the rest of the organism, potentially allowing for very high editing rates. In addition, the experiments below illustrate that the disclosed technique is safe and effective. The technique appears to exhibit no cytotoxicity over a range of doses, allows for normal cell morphology and division, and allows for hatching of blastocysts from the zona pellucida, all signs of a healthy embryo.

In sum, the disclosed approach provides facile, safe, and highly effective gene editing of embryos in vitro.

### BRIEF DESCRIPTION OF THE DRAWINGS

FIG. 1A is a dot plot showing the percent of editing in murine embryos 120 hours after γPNA/DNA nanoparticle (NP) treatment quantified by droplet digital PCR (ddPCR). Each data point represents gDNA from a single blastocyst, five days after one hour of NP treatment at the single-cell stage. FIG. 1B is a standard curve created by taking known amounts of control genomic DNA mixed in varying ratios and performing whole genome amplification. Control genomic DNA was either from untreated β-thalassemic mice or from untreated sickle cell Townes mice, which have a human β-globin gene but do not contain the β-thalassemia mutation, thus registering as wild-type on a ddPCR assay for β-thalassemia. Whole genome amplified samples then underwent droplet digital PCR for editing readout. Measured values by ddPCR are plotted against expected values.

FIGS. 2A and 2B are bar graphs showing the viability of embryos (% 2 cell development (2A), and % blastocysts (2B)) at various concentrations of γPNA/DNA nanoparticle (NP) treatment.

FIGS. 3A and 3B are male (3A) and female (3B) growth curves for reimplanted mice compared to untreated control mice (“untx”) of similar litter sizes from post-natal day of life p1 to p30. Untreated litter sizes of n<10 were excluded from the study. Mice of similar litter studies were compared to avoid the confounding variable of litter size on individual mouse size. FIGS. 3C and 3D are plots showing the level of gene editing in organs/tissue of heathy pups (3C) and pups undergoing spontaneous resorption in the uterus (3D).

FIG. 4A is a plot comparing gene editing among untreated, naked PNA, naked DNA, naked PNA and DNA, and PNA and DNA packaged in nanoparticles (NPs). FIGS. 4B and 4C are bar graphs showing the % survival of embryos at the 2 cell (4B) and blastocyte (4C) stage following treatment with naked PNA, naked DNA, naked PNA and DNA, and PNA and DNA packaged in nanoparticles (NPs).

### DETAILED DESCRIPTION OF THE INVENTION

### I. Definitions

As used herein, the term “isolated” describes a compound of interest (e.g., either a polynucleotide or a polypeptide) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a nucleic acid can be introduced.

As used herein, “transformed” and “transfected” encompass the introduction of a nucleic acid into a cell.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M⁻¹. Affinities greater than 10⁸ M⁻¹ are preferred.

As used herein, “targeting molecule” is a substance which can direct a particle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

As used herein, the terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes a bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol., 79:315-321 (1990); Kostelny, et al., J. Immunol., 148, 1547-1553 (1992).

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “naked” refers to gene editing active agents such as triplex forming molecule peptide nucleic acid oligomers and/or donor oligonucleotide in the absence of, or otherwise free from, a particle-based delivery system such polymeric nanoparticles.

### II. Methods for Embryonic Gene Editing In Vitro

Methods of embryonic gene editing are provided. The methods most typically include contacting polymeric particle-loaded gene editing technology active agents with an embryo in vitro. In some embodiments, the methods include contacting polymeric particle-loaded gene editing technology active agents with male and/or female gametes in vitro prior to fertilization. In some embodiments, particles are not utilitzed, and thus, the methods include direclty contacting an embryo, or gamates, in vitro with an effective amount of non-enzymatic (e.g., non-nuclease) gene editing active agent(s). The contacting is typically effective to introduce the gene editing technology active agent(s) into the embryo or gamates (i.e., transform or transfect the embryo or gamates).

The gene editing technology active agent is typically non-enzymatic. Although typically discussed herein with respect to triplex-forming molecules in combination with donor oligonucleotides, non-enzymatic gene editing technology active agents include, but are not limited to, triplex-forming molecules, pseudocomplementary oligonucleotides, small fragment homologous replacement (e.g., polynucleotide small DNA fragments (SDFs)), single-stranded oligodeoxynucleotide-mediated gene modification (e.g., ssODN/SSOs) and others described in Sargent, Oligonucleotides, 21(2): 55-75 (2011)), and elsewhere. Some gene editing technologies are used in combination with a donor oligonucleotide. In some embodiments, the gene editing technology is the donor oligonucleotide, which can be used alone to modify genes. Thus, the compositions and methods disclosed herein with respect to triplex-forming molecules and donor oligonucleotides is also expressly disclosed for other non-enzymatic gene editing technologies. Exemplary gene triplex-forming molecule and donor oligonucleotide compositions are disclosed in more detail below.

Off-target sequence modifications are extremely low to undetectable for particle-loaded triplex-forming molecules over nuclease-based gene editing techniques such as zinc-finger nucleases, CRISPR/Cas, and TALENs that have off-target and cytotoxic effects.

The gene editing active agents are typically encapsulated and/or entrapped and/or dispersed in polymeric particle(s). For example, the active agents can be in the same or different particles. Thus, various embodiments can include one, two, three or more active agent nucleic acids encapsulated and/or entrapped and/or dispersed in the same or separate particle(s). When packaged in separate particles, the particles can be of the same or different composition or formulation. Exemplary polymers, particles formed therefrom, and method of making particles are disclosed in more detail blow.

In specific embodiments exemplified below, the nanoparticles are fabricated poly(lactic-co-glycolic acid) (PLGA), which has been used in humans for over 40 years and is well known to be safe. Two active agents are loaded into the nanoparticles: a triplex-forming peptide nucleic acid (PNA) that induces recombination at a specific, targeted site on a chromosome and a donor DNA molecule that contains the desired gene sequence. Once administered, nanoparticles are taken up by cells where they degrade, releasing the loaded PNA and DNA. Once inside a cell, the engineered PNA binds to a specific genomic target site, forming a triplex, which induces DNA repair mechanisms that stimulate the recombination of the short, single stranded donor DNA molecule containing the correct sequence, resulting in site-specific gene correction. Off-target sequence modifications are extremely low to undetectable with PNA technology, which may be an advantage for PLGA PNA/DNA-loaded nanoparticles over nuclease-based gene editing techniques such as zinc-finger nucleases, CRISPR/Cas, and TALENs technologies whose off-target, cytotoxic, and/or immunogenic effects could make them unsafe for in vivo gene editing.

In some embodiments, particles are not utilized. Thus, in some embodiments, the compositions and methods include gene editing active agents in the absence or, or otherwise free from, particles.

The contacting can occur by, for example, introducing the active agent, or active agent-loaded particles into the media bathing the embryo or the male and/or female gametes. Thus, injection, transfection reagents, and other means of facilitating the introduction of nucleic acids into the interior of the embryonic cells is optional and can be completely excluded from the method.

The embryo is typically derived from in vitro fertilization. In vitro fertilization (IVF) is a process of fertilization where an egg is combined with sperm outside the body, in vitro. The process typically involves monitoring and stimulating a woman's ovulatory process, removing an ovum or ova (egg or eggs) from the female's ovaries and letting sperm fertilize them in a liquid in a laboratory. In, for example, human IVF, the fertilized egg (zygote) undergoes in vitro embryo culture for 2-6 days, and is then transferred to the same or another woman's uterus, to establish pregnancy.

In a typical human IVF method, male and female gametes are incubated together at a ratio of about 75,000:1 in a culture media to prompt fertilization. A co-incubation of about 1 to 4 hours is sufficient and perhaps even preferable, though co-incubation periods of as much 16 or 24 hours can also be used. In most cases, the egg will be fertilized during co-incubation and will show two pronuclei. In certain situations, such as low sperm count or motility, a single sperm may be injected directly into the egg using intracytoplasmic sperm injection (ICSI). The fertilized egg is passed to a special growth medium and left for about 48 hours until the egg consists of six to eight cells.

For humans, the main durations of embryo culture are until cleavage stage (day two to four after co-incubation) or the blastocyst stage (day five or six after co-incubation). Embryo culture until the blastocyst stage confers a significant increase in live birth rate per embryo transfer, but also confers a decreased number of embryos available for transfer and embryo cryopreservation, so the cumulative clinical pregnancy rates are increased with cleavage stage transfer. Transfer on day two instead of day three after fertilization has no differences in live birth rate, however, there are significantly higher odds of preterm birth and congenital anomalies among births having from embryos cultured until the blastocyst stage compared with cleavage stage.

In preferred embodiments, the embryo is a single cell embryo (i.e., a zygote), however, the embryo can be contacted with the disclosed compositions any time after fertilization and during the in vitro culture period. Thus, for example, the embryo can be contacted with the disclosed composition on day 0, day 1, day 2, day 3, day 4, day 5, or day 6 after co-incubation. The embryo can have 1, 2, 4, 8, 16, or more cells. The embryo can be a zygote, a morula, or a blastocyst. The embryo can be surrounded by a zona pellucida, or can be hatched through the zona pellucida.

The maternal-to-zygotic transition (MZT, also known as Embryonic Genome Activation) is the stage in embryogensis during which development comes under the exclusive control of the zygotic genome rather than the maternal (egg) genome. The egg contains stored maternal genetic material mRNA which controls embryo development until the onset of MZT. After MZT the diploid embryo takes over genetic control. In some embodiments, the embryo is contacted with the disclosed composition before, during, or after the MZT, or a combination thereof.

In some embodiments, the active agent or active agent-loaded particles are introduced into the media containing the male and/or female gametes prior to fertilization. Thus, in some embodiments, gene editing occurs in male or female gametes prior to fertilization, in the embryo after sperm-egg fusion, or a combination thereof.

The embryo may be from, for example, a human or a domesticated, agricultural, or wild animal. Domesticated animals include, for example, dogs, cats, rabbits, ferrets, guinea pigs, hamsters, pigs, monkeys or other primates, and gerbils. Agricultural animals include, for example, horses, cattle, pigs, sheep, rabbits, chickens, and goats. Cattle includes cows, bulls, steers, and heifers.

The embryo is contacted in vitro with triplex-forming molecules and/or donor oligonucleotides in amounts effective to cause the desired mutations in or adjacent to genes in need of repair or alteration. These cells can be referred to as modified cells or modified embryos.

The results below show that non-enzymatic donor DNA alone and in combination with triplex-forming PNA, either naked (i.e., unpackaged), or packaged in polymeric nanoparticles (NPs) can achieve site-specific, genome editing in single-cell fertilized mouse embryos. At the blastocyst stage five days after treatment, PNA/DNA NP treatment resulted in up to nearly 100% gene correction for some of the doses tested. PNA/DNA NP treatment did not cause significant cytotoxicity and allowed for normal cell morphology, cellular division, and zona hatching at the correct developmental stage.

As illustrated in the experiments below, naked and nanoparticle treatment at the single-cell zygote stage allows for normal physiological cell division with no significant cytotoxicity, and treated embryos can be edited at clinically significant rates. Reimplanted murine embryos are capable of normal growth and division with no observed developmental abnormalities. Therefore, embryonic gene editing with naked donor alone and the combination of PNA/DNA naked, or optionally, but preferably, packaged into NPs is a safe, effective and relatively undemanding alternative that can involve pipetting NPs into the embryo culture media.

Preimplantation genetic screening (PGS) or preimplantation genetic diagnosis (PGD) can be used in combination with the disclosed methods. PGS screens for numeral chromosomal abnormalities while PGD diagnosis the specific molecular defect of the inherited disease. In both PGS and PGD, individual cells from a pre-embryo, or preferably trophectoderm cells biopsied from a blastocyst, are analysed during the IVF process. Before the transfer of a pre-embryo back to a woman's uterus, one or two cells are removed from the pre-embryos (8-cell stage), or preferably from a blastocyst. These cells are then evaluated using, for example, fluorescent in situ hybridization (FISH) for the detection of chromosomal abnormalities (for instance, aneuploidy screening or chromosomal translocations) and polymerase chain reaction (PCR) and/or sequencing to identify genomic mutations or correction thereof. Typically within one to two days, following completion of the evaluation, pre-embryos with the desired genetic characteristics can be transferred back to the woman's uterus. Alternatively, a blastocyst can be cryopreserved via vitrification and transferred at a later date to the uterus. In addition, PGS can significantly reduce the risk of multiple pregnancies because fewer embryos, ideally just one, are needed for implantation.

The methods can be used to induce desired gene modification to, for example, correct disease causing mutations or otherwise change or improve genetically linked characteristics.

For example, embryonic gene editing could be used for the creation of genetic mouse models, improving the output of livestock products, and may have clinical significance during in vitro fertilization (IVF) for the treatment or prevention of human diseases. Target diseases include, but are not limited to monogenic diseases, for example, cystic fibrosis where both parents may be homozygous for a mutation. In some embodiments, the disease is an autosomal dominant disorders such as Huntington's disease or a recessive X-linked disorders such as Duchenne muscular dystrophy or Hemophilia A. Embryonic gene editing may also have applications in the prevention of diseases such as Alzheimer's disease, by modifying the ApoE4 risk allele, or in reduction in susceptibility to HIV infection, by editing the CCR5 receptor. Exemplary preferred target diseases are discussed in more detail below.

Additional compositions and methods that may be useful with the compositions and methods disclosed herein are disclosed in U.S. Published Application No. 2017/0283830, published international application WO 2017/143061, and international application no. PCT/US2018/026116 are all which are specifically incorporated by reference in their entireties.

Much higher editing frequencies were achieved in embryos versus bone marrow stem cells. Results in mouse bone marrow stem cells indicate about 7% gene editing, while in embryos it can be up to nearly 100%.

A. Effective Amounts

The active agent is typically administered to the embryo in an effective amount. Typically, the methods include contacting an embryo with an effective amount of naked or particle-packaged non-enzymatic gene editing active agent such as triplex-forming molecules and/or donor oligonucleotides, alone or in combination with a potentiating agent, to modify the cell's genome. In some embodiments, the method includes contacting multi-cell embryo with an effective amount of naked or particle-packaged triplex-forming molecules and/or donor oligonucleotides, optionally in combination with a potentiating agent, to modify the genomes of a sufficient number of cells to achieve a desired result. The effective amount can be a therapeutically effective amount sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder existing in the embryo or that may or would manifest later in development if not for the composition-mediated gene correction.

Triplex-forming molecules are typically administered in an effective amount to induce formation of a triple helix at a target site in the embryo's genome. An effective amount of triplex-forming molecules may also be an amount effective to increase the rate of recombination of a donor fragment relative to administration of the donor fragment in the absence of the triplex-forming molecules. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids. However, as discussed above, in some embodiments, the active agents or particle-packaged active agents are simply introduced into the media bathing the embryo.

Particle-based compositions can have a defined release profile. When two or more active agents are used, the agents or their particles can have the same targeting agent, or different targeting agents, wherein the different targeting agents are capable of specifically binding to the same or different targets on or in the embryo. When two or more compositions are used, the release profiles for the different active agents can be the same or different.

The compositions can be administered or otherwise contacted with the embryo one or more times. For example, the compositions can be contacted with the embryo one, two, three, four, five, six, seven or more times.

In some embodiments, a potentiating agent is administered to the embryo prior to administration of triplex-forming molecules and/or donor oligonucleotides. The potentiating agent can be administered to the embryo, for example, 1, 2, 3, 4, 5, 6, 8, 10, 12, 18, or 24 hours, or 1, 2, 3, 4, 5, 6, or 7 days, or any combination thereof prior to administration of triplex-forming molecules and/or donor oligonucleotides to the embryo.

In some embodiments, naked or particle-packaged triplex-forming molecules and/or donor oligonucleotides is administered to the embryo prior to administration of a potentiating agent to the embryo. The triplex-forming molecules and/or donor oligonucleotides can be administered to the embryo, for example, 1, 2, 3, 4, 5, 6, 8, 10, 12, 18, or 24 hours, or 1, 2, 3, 4, 5, 6, or 7 days, or any combination thereof prior to administration of the potentiating agent to the embryo.

In some embodiments, the compositions are administered in an amount effective to induce gene modification in at least one target allele to occur at frequency of at least 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% of target cells (e.g., a single cell embryo). In some embodiments, particularly in vitro applications, gene modification occurs in at least one target allele at a frequency of about 0.1-25%, or 0.5-25%, or 1-25% 2-25%, or 3-25%, or 4-25% or 5-25% or 6-25%, or 7-25%, or 8-25%, or 9-25%, or 10-25%, 11-25%, or 12-25%, or 13%-25% or 14%-25% or 15-25%, or 2-20%, or 3-20%, or 4-20% or 5-20% or 6-20%, or 7-20%, or 8-20%, or 9-20%, or 10-20%, 11-20%, or 12-20%, or 13%-20% or 14%-20% or 15-20%, 2-15%, or 3-15%, or 4-15% or 5-15% or 6-15%, or 7-15%, or 8-15%, or 9-15%, or 10-15%, 11-15%, or 12-15%, or 13%-15% or 14%-15%.

In some embodiments, the embryo is a single cell embryo and thus the gene modification can occur in at least one target allele at a frequency of at least 65, 70, 75, 80, 85, 90, 95, or 100%.

In some embodiments, gene modification occurs with low off-target effects. In some embodiments, off-target modification is undetectable using routine analysis. In some embodiments, off-target incidents occur at a frequency of 0-1%, or 0-0.1%, or 0-0.01%, or 0-0.001%, or 0-0.0001%, or 0-0000.1%, or 0-0.000001%. In some embodiments, off-target modification occurs at a frequency that is about 10², 10³, 10⁴, or 10⁵-fold lower than at the target site.

B. Exemplary Dosages

In general, by way of example only, dosage forms useful in the methods can include doses in the range of about 10² to about 10⁵⁰, or about 10⁵ to about 10⁴⁰, or about 10¹⁰ to about 10³⁰, or about 10¹² to about 10²⁰ copies of the gene editing technology active agent per dose. In particular embodiments, about 10¹³, 10¹⁴, 10¹⁵, 10¹⁶, or 10¹⁷ copies of triplex-forming molecules and/or donor oligonucleotides are administered to a subject in need thereof.

In other embodiments, dosages are expressed in moles. For example, in some embodiments, the dose of gene editing technology active agent is about 0.1 nmol to about 100 nmol, or about 0.25 nmol to about 50 nmol, or about 0.5 nmol to about 25 nmol, or about 0.75 nmol to about 7.5 nmol.

In other embodiments, dosages are expressed in molecules per target cells. For example, in some embodiments, the dose of triplex-forming molecules and/or donor oligonucleotides is about 10² to about 10⁵⁰, or about 10⁵ to about 10¹⁵, or about 10⁷ to about 10¹², or about 10⁸ to about 10¹¹ copies of the triplex-forming molecules and/or donor oligonucleotides per target cell.

In other embodiments, dosages are expressed in mg/ml, particularly when expressed as an in vitro dosage of active agent such triplex-forming molecules and/or donor oligonucleotides packaged in a particle with or without functional molecules. Dosages can be, for example 0.005 mg/ml to about 100 mg/ml, or about 0.01 mg/ml to about 50 mg/ml, or about 0.125 mg/ml to about 0.5 mg/ml. The Example below illustrates specific exemplary dosages of 12.5 μg/ml, 25 μg/ml, 50 μg/ml, 100 μg/ml, 200 μg/ml, 300 μg/ml, 400 μg/ml, and 500 μg/ml.

Compositions including a donor oligonucleotide should include an amount of donor fragment effective to recombine at the target site alone or in the presence of triplex-forming molecules.

With respect to potentiating agents, preferably an amount effective to increase gene modification when used in combination with a gene modifying technology, compared to using the gene modifying technology in the absence of the potentiating agent.

Dosage units including an effective amount of the compositions are also provided. The dosage can be lower than the effective dosage of the same composition when administered to treat a fetus, child, or an adult. The dosage can be effective to treat or prevent a disease or disorder in the embryo, or subsequent fetus, or juvenile or adult stemming therefrom.

### III. Particle Delivery Vehicles

The active agents are typically packaged in particle delivery vehicle. Thus, the compositions typically include a biodegradable or bioerodible material in which the active agent nucleic acids are encapsulated and/or entrapped and/or dispersed. Compositions can include a plurality of particles having an active agent encapsulated and/or entrapped and/or dispersed therein, in a pharmaceutically-acceptable carrier.

The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation.

In some embodiments, some of the compositions are packaged in particles and some are not. For example, triplex-forming molecules and/or donor oligonucleotides can be incorporated into particles while a co-administered potentiating factor is not. In some embodiments, triplex-forming molecules and/or donor oligonucleotides and/or a potentiating factor are packaged in particles. Different compositions can be packaged in the same particles or different particles. For example, two or more active agents can be mixed and packaged together. In some embodiments, the different compositions are packaged separately into separate particles wherein the particles are similarly or identically composed and/or manufactured. In some embodiments, the different compositions are packaged separately into separate particles wherein the particles are differentially composed and/or manufactured.

The delivery vehicles can be nanoscale compositions, for example, 0.5 nm up to, but not including, about 1 micron. In some embodiments, and for some uses, the particles can be smaller, or larger. Thus, the particles can be microparticles, supraparticles, etc. For example, particle compositions can be between about 1 micron to about 1000 microns. Such compositions can be referred to as microparticulate compositions.

Nanoparticles generally refers to particles in the range of less than 0.5 nm up to, but not including 1,000 nm. In some embodiments, the nanoparticles have a diameter between 500 nm to less than 0.5 nm, or between 50 and 500 nm, or between 50 and 300 nm. Cellular internalization of polymeric particles can highly dependent upon their size, with nanoparticulate polymeric particles being internalized by cells with much higher efficiency than micoparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

In some embodiments, particularly those in which the particles need not be internalized by cells, the particles can be microparticles. Microparticle generally refers to a particle having a diameter, from about 1 micron to about 100 microns. The particles can also be from about 1 to about 50 microns, or from about 1 to about 30 microns, or from about 1 micron to about 10 microns. The microparticles can have any shape. Microparticles having a spherical shape may be referred to as “microspheres.”

Supraparticles are particles having a diameter above about 100 μm in size. For example, supraparticle may have a diameter of about 100 μm to about 1,000 μm in size.

The particles can have a mean particle size. Mean particle size generally refers to the statistical mean particle size (diameter) of the particles in the composition. Two populations can be said to have a substantially equivalent mean particle size when the statistical mean particle size of the first population of particles is within 20% of the statistical mean particle size of the second population of particles; more preferably within 15%, most preferably within 10%.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

A. Polymer

Particles are can be formed of one or more polymers. Exemplary polymers are discussed below. Copolymers such as random, block, or graft copolymers, or blends of the polymers listed below can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

Copolymers of PEG or derivatives thereof with any of the polymers described below may be used to make the polymeric particles. In certain embodiments, the PEG or derivatives may be located in the interior positions of the copolymer. Alternatively, the PEG or derivatives may locate near or at the terminal positions of the copolymer. For example, one or more of the polymers above can be terminated with a block of polyethylene glycol. In some embodiments, the core polymer is a blend of pegylated polymer and non-pegylated polymer, wherein the base polymer is the same (e.g., PLGA and PLGA-PEG) or different (e.g., PLGA-PEG and PLA). In certain embodiments, the microparticles or nanoparticles are formed under conditions that allow regions of PEG to phase separate or otherwise locate to the surface of the particles. The surface-localized PEG regions alone may perform the function of, or include, the surface-altering agent. In particular embodiments, the particles are prepared from one or more polymers terminated with blocks of polyethylene glycol as the surface-altering material.

Release

In certain embodiments, the particles in amniotic space release therapeutic agents over at least 3 days, 5 days, 1 week, 2 weeks, 3 weeks, 1 month, 2 months, 3 months, or longer.

Surface Charge

In certain embodiments, the particles possess a ζ-potential of between about 50 mV and about −50 mV, between about 30 mV and about −30 mV, or between about 10 mV and about −10 mV.

Some charges on the surface of the particles may facilitate binding with the MMC defect in amniotic space or binding to nucleic acids. Thus, the charge of the particles can be selected based on the active agent to be delivered, the disease to be treated, or a combination thereof.

In some embodiments, it may be desirable for the particles to have a negative charge, for example when delivering proteins such as growth factors. Thus, some or all of the polymers forming the particle can have a terminal moiety that imparts a negative charge to the particle. In some embodiments, the negatively charged moiety is a chemical modification to the polymer itself that imparts a negative charge to the polymer. In some embodiments, the negatively charge moiety is a separate, negatively-charged component that is conjugated to the polymer.

The terminal moiety that imparts a negative charge to the particles can be an acidic group or an anionic group. Examples of acidic groups include, but are not limited to, carboxylic acids, protonated sulfates, protonated sulfonates, protonated phosphates, singly- or doubly protonated phosphonates, and singly- or doubly protonated hydroxamates. The corresponding salts of these acidic groups form anionic groups such as carboxylates, sulfates, sulfonates, singly- or doubly deprotonated phosphates, singly- or doubly deprotonated phosphonates, and hydroxamates.

In some embodiments, the particles may be used as nucleic acid carriers. In these embodiments, the particles can be formed of one or more cationic polymers which complex with one or more nucleic acids which are negatively charged.

The cationic polymer can be any synthetic or natural polymer bearing at least two positive charges per molecule and having sufficient charge density and molecular size to bind to nucleic acid under physiological conditions (i.e., pH and salt conditions encountered within the body or within cells). In certain embodiments, the polycationic polymer contains one or more amine residues.

Suitable cationic polymers include, for example, polyethylene imine (PEI), polyallylamine, polyvinylamine, polyvinylpyridine, aminoacetalized poly(vinyl alcohol), acrylic or methacrylic polymers (for example, poly(N,N-dimethylaminoethylmethacrylate)) bearing one or more amine residues, polyamino acids such as polyornithine, polyarginine, and polylysine, protamine, cationic polysaccharides such as chitosan, DEAE-cellulose, and DEAE-dextran, and polyamidoamine dendrimers (cationic dendrimer), as well as copolymers and blends thereof. In some embodiments, the polycationic polymer is poly(amine-co-ester), poly(amine-co-amide) polymer, or poly(amine-co-ester-co-ortho ester).

Cationic polymers can be either linear or branched, can be either homopolymers or copolymers, and when containing amino acids can have either L or D configuration, and can have any mixture of these features. Preferably, the cationic polymer molecule is sufficiently flexible to allow it to form a compact complex with one or more nucleic acid molecules.

In some embodiments, the cationic polymer has a molecular weight of between about 5,000 Daltons and about 100,000 Daltons, more preferably between about 5,000 and about 50,000 Daltons, most preferably between about 10,000 and about 35,000 Daltons.

In particular embodiments, the particles include a hydrophobic polymer, poly(amine-co-ester), poly(amine-co-amide) polymer, or poly(amine-co-ester-co-ortho ester), and optionally, but a shell of, for example, PEG. The core-shell particles can be formed by a co-block polymer. Exemplary polymers are provided below.

1. Exemplary Polymers Hydrophobic Polymers

The polymer that forms the core of the particle may be any biodegradable or non-biodegradable synthetic or natural polymer. In a preferred embodiment, the polymer is a biodegradable polymer.

Particles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Any number of biocompatible polymers can be used to prepare the particles. In one embodiment, the biocompatible polymer(s) is biodegradable. In another embodiment, the particles are non-degradable. In other embodiments, the particles are a mixture of degradable and non-degradable particles.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11(1):82-90 (2011), Tietjen, et al. Nature Communications, 8:191 (2017) doi:10.1038/s41467-017-00297-x, and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Exemplary polymers include, but are not limited to, cyclodextrin-containing polymers, in particular cationic cyclodextrin-containing polymers, such as those described in U.S. Pat. No. 6,509,323,

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylenes such as polyethylene and polypropylene, polyalkylene glycols such as poly(ethylene glycol) (PEG), polyalkylene oxides (PEO), polyalkylene terephthalates such as poly(ethylene terephthalate) and ethylene vinyl acetate polymer (EVA), polyvinyl alcohols, polyvinyl ethers, polyvinyl esters such as poly(vinyl acetate), polyethylene, polypropylene, poly(vinyl acetate), poly vinyl chloride, polystyrene, polyvinyl halides such as poly(vinyl chloride) (PVC), polyvinylpyrrolidone, polysiloxanes, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, hydroxypropylcellulose, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), Poly(2-hydroxyethyl methacrylate) (pHEMA), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as co-polymers.

The polymer may be a bioadhesive polymer that is hydrophilic or hydrophobic. Hydrophilic polymers include CARBOPOL™ (a high molecular weight, crosslinked, acrylic acid-based polymers such as those manufactured by NOVEON™), polycarbophil, cellulose esters, and dextran. polymers of acrylic acids, include, but are not limited to, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”).

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and 5100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

In certain embodiments, the hydrophobic polymer is an aliphatic polyester. In preferred embodiments, the hydrophobic polymer is polyhydroxyester such as poly(lactic acid), poly(glycolic acid), or poly(lactic acid-co-glycolic acid).

Other polymers include, but are not limited to, polyalkyl cyanoacralate, polyamino acids such as poly-L-lysine (PLL), poly(valeric acid), and poly-L-glutamic acid, hydroxypropyl methacrylate (HPMA), polyorthoesters, poly(ester amides), poly(ester ethers), polydioxanone and its copolymers, polyhydroxyalkanoates, polypropylene fumarate, polyoxymethylene, poly(butyric acid), trimethylene carbonate, and polyphosphazenes.

The particles can be designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. The hydrophobic poly (lactic acid) (PLA), more hydrophilic poly (glycolic acid) PGA and their copolymers, poly (lactide-co-glycolide) (PLGA) have different release rates. The degradation rate of these polymers, and often the corresponding drug release rate, can vary from days (PGA) to months (PLA) and is easily manipulated by varying the ratio of PLA to PGA.

In some preferred embodiments, the particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(8-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. For example, particles can also contain one or more polymer conjugates containing end-to-end linkages between the polymer and a targeting moiety or a detectable label. For example, a modified polymer can be a PLGA-PEG-peptide block polymer.

The in vivo stability/release of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

A shell can also be formed of or contain a hyperbranched polymer (HP) with hydroxyl groups, such as a hyperbranched polyglycerol (HPG), hyperbranched peptides (HPP), hyperbranched oligonucleotides (HON), hyperbranched polysaccharides (HPS), and hyperbranched polyunsaturated or saturated fatty acids (HPF). The HP can be covalently bound to the one or more materials that form the core such that the hydrophilic HP is oriented towards the outside of the particles and the hydrophobic material oriented to form the core.

The HP coating can be modified to adjust the properties of the particles. For example, unmodified HP coatings impart stealth properties to the particles which resist non-specific protein absorption and are referred to as nonbioadhesive nanoparticles (NNPs). Alternatively, the hydroxyl groups on the HP coating can be chemically modified to form functional groups that react with functional groups on tissue or otherwise interact with tissue to adhere the particles to the tissue, cells, or extracellular materials, such as proteins. Such functional groups include, but are not limited to, aldehydes, amines, and O-substituted oximes. Particles with an HP coating chemically modified to form functional groups are referred to as bioadhesive nanoparticles (BNPs). The chemically modified HP coating of BNPs forms a bioadhesive corona of the particle surrounding the hydrophobic material forming the core. See, for example, WO 2015/172149, WO 2015/172153, WO 2016/183209, and U.S. Published Applications 2017/0000737 and 2017/0266119.

Particles can be formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 μm) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

Exemplary particles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

2. Poly(Amine-Co-Esters), Poly(Amine-Co-Amides), and Poly(Amine-Co-Ester-Co-Ortho Esters)

The core of the particles can be formed of or contain one or more poly(amine-co-ester), poly(amine-co-amide), poly(amine-co-ester-co-ortho ester) or a combination thereof. In some embodiments, the particles are polyplexes. In some embodiments, the content of a hydrophobic monomer in the polymer is increased relative the content of the same hydrophobic monomer when used to form polyplexes. Increasing the content of a hydrophobic monomer in the polymer forms a polymer that can form solid core particles in the presence of nucleic acids. Unlike polyplexes, these particles are stable for long periods of time during incubation in buffered water, or serum, or upon administration (e.g., injection) into animals. They also provide for a sustained release of nucleic acids which leads to long term activity. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa.

The polymers can have the general formula:

((A)ₓ-(B)_{y}-(C)_{q}-(D)_{w}-(E)_{f})ₕ,

wherein A, B, C, D, and E independently include monomeric units derived from lactones (such as pentadecalactone), a polyfunctional molecule (such as N-methyldiethanolamine), a diacid or diester (such as diethylsebacate), an ortho ester, or polyalkylene oxide (such as polyethylene glycol). In some aspects, the polymers include at least a lactone, a polyfunctional molecule, and a diacid or diester monomeric units. In some aspects, the polymers include at least a lactone, a polyfunctional molecule, an ortho ester, and a diacid or diester monomeric units. In general, the polyfunctional molecule contains one or more cations, one or more positively ionizable atoms, or combinations thereof. The one or more cations are formed from the protonation of a basic nitrogen atom, or from quaternary nitrogen atoms.

In general, x, y, q, w, and f are independently integers from 0-1000, with the proviso that the sum (x+y+q+w+f) is greater than one. h is an integer from 1 to 1000.

In some forms, the percent composition of the lactone can be between about 30% and about 100%, calculated as the mole percentage of lactone unit vs. (lactone unit+diester/diacid). Expressed in terms of molar ratio, the lactone unit vs. (lactone unit+diester/diacid) content is between about 0.3 and about 1. Preferably, the number of carbon atoms in the lactone unit is between about 10 and about 24. In some embodiments, the number of carbon atoms in the lactone unit is between about 12 and about 16. In some embodiments, the number of carbon atoms in the lactone unit is 12 (dodecalactone), 15 (pentadecalactone), or 16 (hexadecalactone).

The molecular weight of the lactone unit in the polymer, the lactone unit's content of the polymer, or both, influences the formation of solid core particles.

Suitable polymers as well as particles and polyplexes formed therefrom are disclosed in WO 2013/082529, WO 2016/183217, U.S. Published Application No. 2016/0251477, U.S. Published Application No. 2015/0073041, U.S. Published Application No. 2014/0073041, and U.S. Pat. No. 9,272,043, each of which is specifically incorporated by reference in entirety.

For example, in some embodiments, the polymer includes a structure that has the formula:

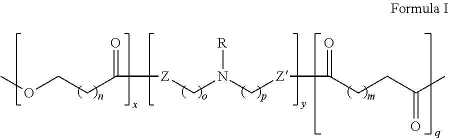

wherein n is an integer from 1-30, m, o, and p are independently an integer from 1-20, x, y, and q are independently integers from 1-1000, Z and Z′ are independently O or NR′, wherein R and R′ are independently hydrogen, substituted or unsubstituted alkyl, or substituted or unsubstituted aryl. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc. In particular embodiments, the values of x, y, and q are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. The polymer can be prepared from one or more lactones, one or more amine-diols, triamines, or hydroxy diamines, and one or more diacids or diesters. In those embodiments where two or more different lactone, diacid or diester, and/or triamine, amine-diol, or hydroxy diamine monomers are used, the values of n, o, p, and/or m can be the same or different.

In some forms, the percent composition of the lactone unit is between about 30% and about 100%, calculated lactone unit vs. (lactone unit+diester/diacid). Expressed in terms of a molar ratio, the lactone unit vs. (lactone unit+diester/diacid) content is between about 0.3 and about 1, i.e; x/(x+q) is between about 0.3 and about 1. Preferably, the number of carbon atoms in the lactone unit is between about 10 and about 24, more preferably the number of carbon atoms in the lactone unit is between about 12 and about 16. Most preferably, the number of carbon atoms in the lactone unit is 12 (dodecalactone), 15 (pentadecalactone), or 16 (hexadecalactone).

In some embodiments, Z and Z′ are O. In some embodiments, Z is O and Z′ is NR′, or Z is NR′ and Z′ is O, wherein R′ is hydrogen, substituted or unsubstituted alkyl, or substituted or unsubstituted aryl. Examples of R′ include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

In some embodiments, Z and Z′ are O and n is an integer from 1-24, such 4, 10, 13, or 14.

In some embodiments, Z and Z′ are O, n is an integer from 1-24, such 4, 10, 13, or 14, and m is an integer from 1-10, such as 4, 5, 6, 7, or 8.

In some embodiments, Z and Z′ are O, n is an integer from 1-24, such 4, 10, 13, or 14, m is an integer from 1-10, such as 4, 5, 6, 7, or 8, and o and p are the same integer from 1-6, such 2, 3, or 4.

In some embodiments, Z and Z′ are O, n is an integer from 1-24, such 4, 10, 13, or 14, m is an integer from 1-10, such as 4, 5, 6, 7, or 8, and R is alkyl, such as methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, or aryl, such as phenyl, naphthalyl, anthracenyl, phenanthryl, chrysenyl, pyrenyl, tolyl, or xylyl.

In certain embodiments, n is 14 (e.g., pentadecalactone, PDL), m is 7 (e.g., diethylsebacate, DES), o and p are 2 (e.g., N-methyldiethanolamine, MDEA). In certain embodiments, n, m, o, and p are as defined above, and PEG is incorporated as a monomer.

In particular embodiments, the values of x, y, and q are such that the weight average molecular weight of the polymer is greater than 5,000 Daltons.

The polymer can be prepared from one or more substituted or unsubstituted lactones, one or more substituted or unsubstituted amine-diols (Z and Z′═O), triamines (Z and Z′═NR′), or hydroxy-diamines (Z═O, and Z′═NR′, or vice versa) and one or more substituted or unsubstituted diacids or diesters. In those embodiments where two or more different lactone, diacid or diester, and/or triamine, amine-diol, or hydroxy diamine monomers are used, than the values of n, o, p, and/or m can be the same or different.

The monomer units can be substituted at one or more positions with one or more substituents. Exemplary substituents include, but are not limited to, alkyl groups, cyclic alkyl groups, alkene groups, cyclic alkene groups, alkynes, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

The polymer is preferably biocompatible. Readily available lactones of various ring sizes are known to possess low toxicity: for example, polyesters prepared from small lactones, such as poly(caprolactone) and poly(p-dioxanone) are commercially available biomaterials which have been used in clinical applications. Large (e.g., C₁₆-C₂₄) lactones and their polyester derivatives are natural products that have been identified in living organisms, such as bees. Lactones containing ring carbon atoms between 16 and 24 are specifically contemplated and disclosed.

In other embodiments, the polymer is biocompatible and biodegradable. The nucleic acid(s) encapsulated by and/or associated with the particles can be released through different mechanisms, including diffusion and degradation of the polymeric matrix. The rate of release can be controlled by varying the monomer composition of the polymer and thus the rate of degradation. For example, if simple hydrolysis is the primary mechanism of degradation, increasing the hydrophobicity of the polymer may slow the rate of degradation and therefore increase the time period of release. In all case, the polymer composition is selected such that an effective amount of nucleic acid(s) is released to achieve the desired purpose/outcome.

The polymers can further include one or more blocks of an alkylene oxide, such as polyethylene oxide, polypropylene oxide, and/or polyethylene oxide-co-polypropylene oxide. The structure of a PEG-containing polymer is shown below:

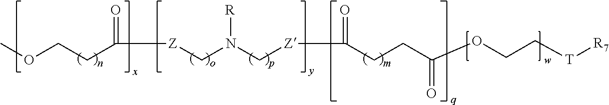

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R7 is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The structure of a PEG-containing copolymer is shown below:

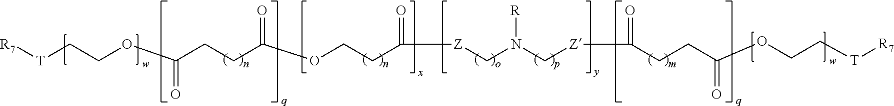

wherein n is an integer from 1-30, m, o, and p are independently an integer from 1-20, x, y, q, and w are independently integers from 1-1000, Z and Z′ are independently O or NR′, wherein R and R′ are independently hydrogen, substituted or unsubstituted alkyl, or substituted or unsubstituted aryl, wherein T is oxygen or is absent, and wherein R7 is hydrogen, alkyl, substituted alkyl, aryl, substituted alkyl, cycloalkyl, substituted cycloalkyl, maleimide, amine, thiol, N-hydroxysuccinimide ester, azide, acrylate, methacrylate, alkyne, hydroxide, or isocynate. In particular embodiments, the values of x, y, q, and w are such that the weight average molecular weight of the polymer is greater than 5,000 Daltons. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa. Examples of R and R′ groups include, but are not limited to, hydrogen, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl, phenyl, naphthalyl, anthracenyl, phenanthryl, chrysenyl, pyrenyl, tolyl, xylyl, etc.

The blocks of polyalkylene oxide can located at the termini of the polymer (i.e., by reacting PEG having one hydroxy group blocked, for example, with a methoxy group), within the polymer backbone (i.e., neither of the hydroxyl groups are blocked), or combinations thereof.

In particular embodiments, the synthetic polymer includes polymers or copolymers of lactic acid, glycolic acid, degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), poly(amine-co-ester) polymers, or a combination of any two or more of the foregoing. In specific embodiments, the particles are nanoparticles formed of PLGA poly(lactic-co-glycolic acid) (PLGA), a blend of PLGA and poly(beta-amino) esters (PBAEs) (e.g., about 5 and about 25 percent PBAE (wt %)), or poly(amine-co-ester) (PACE). In some embodiments, these particles are utilized for intracellular delivery of gene editing compositions such as peptide nucleic acids alone or in combination with donor oligonucleotides.

In other particular embodiments, the particles are formed of hydrogel type materials such as alginate, chitosan, poly(HEMA) and other acrylate polymers and copolymers. In specific embodiments, the particles are microparticles formed of alginate. In some embodiments, these particles are utilized for extracellular delivery (e.g., paracrine delivery) of a growth factor such a FGF.

B. Polycations

In some embodiments, the nucleic acids are complexed to polycations to increase the encapsulation efficiency of the nucleic acids into the particles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In one embodiment, the polycation is a polyamine Polyamines are compounds having two or more primary amine groups. In a preferred embodiment, the polyamine is a naturally occurring polyamine that is produced in prokaryotic or eukaryotic cells. Naturally occurring polyamines represent compounds with cations that are found at regularly-spaced intervals and are therefore particularly suitable for complexing with nucleic acids. Polyamines play a major role in very basic genetic processes such as DNA synthesis and gene expression. Polyamines are integral to cell migration, proliferation and differentiation in plants and animals. The metabolic levels of polyamines and amino acid precursors are critical and hence biosynthesis and degradation are tightly regulated. Suitable naturally occurring polyamines include, but are not limited to, spermine, spermidine, cadaverine and putrescine. In a preferred embodiment, the polyamine is spermidine.

In another embodiment, the polycation is a cyclic polyamine Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane), which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

In some embodiments, the particles themselves are a polycation (e.g., a blend of PLGA and poly(beta amino ester).

C. Coupling Agents or Ligands

The external surface of the polymeric particles may be modified by conjugating to, or incorporating into, the surface of the particle a coupling agent or ligand.

In a preferred embodiment, the coupling agent is present in high density on the surface of the particle. As used herein, “high density” refers to polymeric particles having a high density of ligands or coupling agents, which is preferably in the range of 1,000 to 10,000,000, more preferably 10,000-1,000,000 ligands per square micron of particle surface area. This can be measured by fluorescence staining of dissolved particles and calibrating this fluorescence to a known amount of free fluorescent molecules in solution.

Coupling agents associate with the polymeric particles and provide substrates that facilitate the modular assembly and disassembly of functional elements to the particles. Coupling agents or ligands may associate with particles through a variety of interactions including, but not limited to, hydrophobic interactions, electrostatic interactions and covalent coupling.

In a preferred embodiment, the coupling agents are molecules that match the polymer phase hydrophile-lipophile balance. Hydrophile-lipophile balances range from 1 to 15. Molecules with a low hydrophile-lipophile balance are more lipid loving and thus tend to make a water in oil emulsion while those with a high hydrophile-lipophile balance are more hydrophilic and tend to make an oil in water emulsion. Fatty acids and lipids have a low hydrophile-lipophile balance below 10.

Any amphiphilic polymer with a hydrophile-lipophile balance in the range 1-10, more preferably between 1 and 6, most preferably between 1 and up to 5, can be used as a coupling agent. Examples of coupling agents which may associate with polymeric particles via hydrophobic interactions include, but are not limited to, fatty acids, hydrophobic or amphipathic peptides or proteins, and polymers. These classes of coupling agents may also be used in any combination or ratio. In a preferred embodiment, the association of adaptor elements with particles facilitates a prolonged presentation of functional elements, which can last for several weeks.

Coupling agents can also be attached to polymeric particles through covalent interactions through various functional groups. Functionality refers to conjugation of a molecule to the surface of the particle via a functional chemical group (carboxylic acids, aldehydes, amines, sulfhydryls and hydroxyls) present on the surface of the particle and present on the molecule to be attached.

Functionality may be introduced into the particles in two ways. The first is during the preparation of the particles, for example during the emulsion preparation of particles by incorporation of stabilizers with functional chemical groups. Suitable stabilizers include hydrophobic or amphipathic molecules that associate with the outer surface of the particles.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a molecule such as a protein. The reaction is an N-nucleophilic substitution and results in a stable N-alkylcarbamate linkage of the molecule to the polymer. The “coupling” of the molecule to the “activated” polymer matrix is maximal in the pH range of 9-10 and normally requires at least 24 hrs. The resulting molecule-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of molecules in a totally aqueous environment at the physiological pH of 7.0. Briefly, EDAC and sulfo-NHS form an activated ester with the carboxylic acid groups of the polymer which react with the amine end of a molecule to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the molecule-polymer complex.

By using either of these protocols it is possible to “activate” almost all polymers containing either hydroxyl or carboxyl groups in a suitable solvent system that will not dissolve the polymer matrix.

A useful coupling procedure for attaching molecules with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines Activation and coupling take place at pH 11. The linkage is stable in the pH range from 1-8 and is suitable for transit through the intestine.

Any suitable coupling method known to those skilled in the art for the coupling of molecules and polymers with double bonds, including the use of UV crosslinking, may be used for attachment of molecules to the polymer.

In one embodiment, coupling agents can be conjugated to affinity tags. Affinity tags are any molecular species which form highly specific, noncovalent, physiochemical interactions with defined binding partners. Affinity tags which form highly specific, noncovalent, physiochemical interactions with one another are defined herein as “complementary”. Suitable affinity tag pairs are well known in the art and include epitope/antibody, biotin/avidin, biotin/streptavidin, biotin/neutravidin, glutathione-S-transferase/glutathione, maltose binding protein/amylase and maltose binding protein/maltose. Examples of suitable epitopes which may be used for epitope/antibody binding pairs include, but are not limited to, HA, FLAG, c-Myc, glutatione-S-transferase, His₆, GFP, DIG, biotin and avidin. Antibodies (both monoclonal and polyclonal and antigen-binding fragments thereof) which bind to these epitopes are well known in the art.

Affinity tags that are conjugated to coupling agents allow for highly flexible, modular assembly and disassembly of functional elements which are conjugated to affinity tags which form highly specific, noncovalent, physiochemical interactions with complementary affinity tags which are conjugated to coupling agents. Adaptor elements may be conjugated with a single species of affinity tag or with any combination of affinity tag species in any ratio. The ability to vary the number of species of affinity tags and their ratios conjugated to adaptor elements allows for exquisite control over the number of functional elements which may be attached to the particles and their ratios.

In another embodiment, coupling agents are coupled directly to functional elements in the absence of affinity tags, such as through direct covalent interactions. Coupling agents can be covalently coupled to at least one species of functional element. Coupling agents can be covalently coupled to a single species of functional element or with any combination of species of functional elements in any ratio.

In a preferred embodiment, coupling agents are conjugated to at least one affinity tag that provides for assembly and disassembly of modular functional elements which are conjugated to complementary affinity tags. In a more preferred embodiment, coupling agents are fatty acids that are conjugated with at least one affinity tag. In a particularly preferred embodiment, the coupling agents are fatty acids conjugated with avidin or streptavidin. Avidin/streptavidin-conjugated fatty acids allow for the attachment of a wide variety of biotin-conjugated functional elements.

The coupling agents are preferably provided on, or in the surface of, particles at a high density. This high density of coupling agents allows for coupling of the polymeric particles to a variety of species of functional elements while still allowing for the functional elements to be present in high enough numbers to be efficacious.

1. Fatty Acids

The coupling agents may include fatty acids. Fatty acids may be of any acyl chain length and may be saturated or unsaturated. In a particularly preferred embodiment, the fatty acid is palmitic acid. Other suitable fatty acids include, but are not limited to, saturated fatty acids such as butyric, caproic, caprylic, capric, lauric, myristic, stearic, arachidic and behenic acid. Still other suitable fatty acids include, but are not limited to, unsaturated fatty acids such as oleic, linoleic, alpha-linolenic, arachidonic, eicosapentaenoic, docosahexaenoic and erucic acid.

2. Hydrophobic or Amphipathic Peptides

The coupling agents may include hydrophobic or amphipathic peptides. Preferred peptides should be sufficiently hydrophobic to preferentially associate with the polymeric particle over the aqueous environment Amphipathic polypeptides useful as adaptor elements may be mostly hydrophobic on one end and mostly hydrophilic on the other end. Such amphipathic peptides may associate with polymeric particles through the hydrophobic end of the peptide and be conjugated on the hydrophilic end to a functional group.

3. Hydrophobic Polymers

Coupling agents may include hydrophobic polymers. Examples of hydrophobic polymers include, but are not limited to, polyanhydrides, poly(ortho)esters, and polyesters such as polycaprolactone.

### IV. Non-Enzymatic Gene Editing Active Agents

Non-nuclease based gene editing technologies include, but are not limited to, triplex-forming, pseudocomplementary oligonucleotides, small fragment homologous replacement (e.g., polynucleotide small DNA fragments (SDFs)), single-stranded oligodeoxynucleotide-mediated gene modification (e.g., ssODN/SSOs) and other described in Sargent, Oligonucleotides, 21(2): 55-75 (2011)), and elsewhere. Some gene editing technologies are used in combination with a donor oligonucleotide. In some embodiments, the gene editing technology is the donor oligonucleotide, which can be used alone to modify genes.

A. Triplex-Forming Molecules (TFMs)

1. Compositions

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA). The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids (PNAs). Triplex-forming molecules are described in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011). As discussed in more detail below, triplex-forming molecules are typically single-stranded oligonucleotides that bind to polypyrimidine:polypurine target motif in a double stranded nucleic acid molecule to form a triple-stranded nucleic acid molecule. The single-stranded oligonucleotide/oligomer typically includes a sequence substantially complementary to the polypurine strand of the polypyrimidine:polypurine target motif via Hoogsteen or reverse Hoogsteen binding.

a. Triplex-Forming Oligonucleotides (TFOs)

Triplex-forming oligonucleotides (TFOs) are defined as oligonucleotides which bind as third strands to duplex DNA in a sequence specific manner The oligonucleotides are synthetic or isolated nucleic acid molecules which selectively bind to or hybridize with a predetermined target sequence, target region, or target site within or adjacent to a human gene so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The nucleobase (sometimes referred to herein simply as “base”) composition may be homopurine or homopyrimidine. Alternatively, the nucleobase composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleobase sequence of the oligonucleotides/oligomer is selected based on the sequence of the target sequence, the physical constraints imposed by the need to achieve binding of the oligonucleotide/oligomer within the major groove of the target region, and the need to have a low dissociation constant (K_{d}) for the oligo/target sequence complex. The oligonucleotides/oligomers have a nucleobase composition which is conducive to triple-helix formation and is generated based on one of the known structural motifs for third strand binding (e.g. Hoogsteen binding). The most stable complexes are formed on polypurine:polypyrimidine elements, which are relatively abundant in mammalian genomes. Triplex formation by TFOs can occur with the third strand oriented either parallel or anti-parallel to the purine strand of the nucleic acid duplex. In the anti-parallel, purine motif, the triplets are G.G:C and A.A:T, whereas in the parallel pyrimidine motif, the canonical triplets are C⁺.G:C and T.A:T. The triplex structures can be stabilized by one, two or three Hoogsteen hydrogen bonds (depending on the nucleobase) between the bases in the TFO strand and the purine strand in the duplex. A review of base compositions and binding properties for third strand binding oligonucleotides and/or peptide nucleic acids is provided in, for example, U.S. Pat. No. 5,422,251, Bentin et al., Nucl. Acids Res., 34(20): 5790-5799 (2006), and Hansen et al., Nucl. Acids Res., 37(13): 4498-4507 (2009).

Preferably, the oligonucleotide/oligomer binds to or hybridizes to the target sequence under conditions of high stringency and specificity. Most preferably, the oligonucleotides/oligomers bind in a sequence-specific manner within the major groove of duplex DNA. Reaction conditions for in vitro triple helix formation of an oligonucleotide/oligomer to a double stranded nucleic acid sequence vary from oligo to oligo, depending on factors such as polymer length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex-forming molecule is said to be substantially complementary to a target region when the oligonucleotide has a nucleobase composition which allows for the formation of a triple-helix with the target region. As such, an oligonucleotide/oligomer can be substantially complementary to a target region even when there are non-complementary bases present in the oligonucleotide/oligomer. As stated above, there are a variety of structural motifs available which can be used to determine the nucleobase sequence of a substantially complementary oligonucleotide/oligomer.

b. Peptide Nucleic Acids (PNA)

In another embodiment, the triplex-forming molecules are peptide nucleic acids (PNAs). Peptide nucleic acids can be considered polymeric molecules in which the sugar phosphate backbone of an oligonucleotide has been replaced in its entirety by repeating substituted or unsubstituted N-(2-aminoethyl)-glycine residues that are linked by amide bonds. The various nucleobases are linked to the backbone by methylene carbonyl linkages. PNAs maintain spacing of the nucleobases in a manner that is similar to that of an oligonucleotide (DNA or RNA), but because the sugar phosphate backbone has been replaced, classic (unsubstituted) PNAs are achiral and neutrally charged molecules. Peptide nucleic acids are composed of peptide nucleic acid residues (sometimes referred to as ‘residues’). The nucleobases can be any of the standard bases (uracil, thymine, cytosine, adenine and guanine) or any of the modified heterocyclic nucleobases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin et al., Nucl. Acids Res., 34(20): 5790-5799 (2006) and Hansen et al., Nucl. Acids Res., 37(13): 4498-4507 (2009)), or two PNA molecules linked together by a linker of sufficient flexibility to form a single bis-PNA molecule (See: U.S. Pat. No. 6,441,130). In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker residues in any combination of two or more of the foregoing.

PNAs can also include other positively charged moieties to increase the solubility of the PNA and increase the affinity of the PNA for duplex DNA. Commonly used positively charged moieties include the amino acids lysine and arginine (e.g., as additional substituents attached to the C- or N-terminus of the PNA oligomer (or a segment thereof) or as a side-chain modification of the backbone (see Huang et al., Arch. Pharm. Res. 35(3): 517-522 (2012) and Jain et al., JOC, 79(20): 9567-9577 (2014)), although other positively charged moieties may also be useful (See for Example: U.S. Pat. No. 6,326,479). In some embodiments, the PNA oligomer can have one or more ‘miniPEG’ side chain modifications of the backbone (see, for example, U.S. Pat. No. 9,193,758 and Sahu et al., JOC, 76: 5614-5627 (2011)).

Peptide nucleic acids are unnatural synthetic polyamides, prepared using known methodologies, generally as adapted from peptide synthesis processes.

c. Tail Clamp Peptide Nucleic Acids (tcPNA)

Although polypurine:polypyrimidine stretches do exist in mammalian genomes, it is desirable to target triplex formation in the absence of this requirement. In some embodiments such as PNA, triplex-forming molecules include a “tail” added to the end of the Watson-Crick binding portion. Adding additional nucleobases, known as a “tail” or “tail clamp”, to the Watson-Crick binding portion that bind to the target strand outside the triple helix further reduces the requirement for a polypurine:polypyrimidine stretch and increases the number of potential target sites. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42(47):13996-4003 (2003); Bentin, et al., Biochemistry, 42(47):13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99(26):16695-700 (2002)).

Tails added to clamp PNAs (sometimes referred to as bis-PNAs) form tail-clamp PNAs (referred to as tcPNAs) that have been described by Kaihatsu, et al., Biochemistry, 42(47):13996-4003 (2003); Bentin, et al., Biochemistry, 42(47):13987-95 (2003). tcPNAs are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared to PNA without the tail.

In some embodiments a PNA tail clamp system includes:

a) optionally, a positively charged region having a positively charged amino acid subunit, e.g., a lysine subunit;

b) a first region including a plurality of PNA subunits having Hoogsteen homology with a target sequence;

c) a second region including a plurality of PNA subunits having Watson Crick homology binding with the target sequence;

d) a third region including a plurality of PNA subunits having Watson Crick homology binding with a tail target sequence;

e) optionally, a second positively charged region having a positively charged amino acid subunit, e.g., a lysine subunit.

In some embodiments, a linker is disposed between b) and c). In some embodiments, one or more PNA subunits of the tail clamp is modified as disclosed herein.

d. PNA Modifications

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but often result in reduced binding affinity and/or sequence specificity.

Triplex-forming peptide nucleic acid (PNA) oligomers having a γ (also referred to as “gamma”) modification (also referred to as “substitution”) in one or more PNA residues (also referred to as “subunits”) of the PNA oligomer are also provided. In some embodiments, the some or all of the PNA residues are modified at the gamma position in the polyamide backbone (γPNAs) as illustrated below (wherein “B” is a nucleobase and “R” is a substitution at the gamma position).

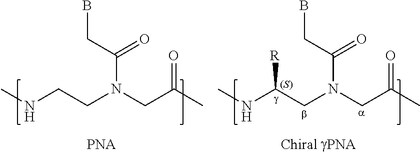

Substitution at the gamma position creates chirality and provides helical pre-organization to the PNA oligomer, yielding substantially increased binding affinity to the target DNA (Rapireddy, et al., Biochemistry, 50(19):3913-8 (2011), He et al., “The Structure of a γ-modified peptide nucleic acid duplex”, Mol. BioSyst. 6:1619-1629 (2010); and Sahu et al., “Synthesis and Characterization of Conformationally Preorganized, (R)-Diethylene Glycol-Containing γ-Peptide Nucleic Acids with Superior Hybridization Properties and Water Solubility”, J. Org. Chem, 76:5614-5627) (2011)). Other advantageous properties can be conferred depending on the chemical nature of the specific substitution at the gamma position (the “R” group in the illustration of the Chiral γPNA, above).

One class of γ substitution, is miniPEG, but other residues and side chains can be considered, and even mixed substitutions can be used to tune the properties of the oligomers. “MiniPEG” and “MP” refers to diethylene glycol. MiniPEG-containing γPNAs are conformationally preorganized PNAs that exhibit superior hybridization properties and water solubility as compared to the original PNA design and other chiral γPNAs. Sahu et al., describes γPNAs prepared from L-amino acids that adopt a right-handed helix, and γPNAs prepared from D-amino acids that adopt a left-handed helix. Only the right-handed helical γPNAs hybridize to DNA or RNA with high affinity and sequence selectivity. In the most preferred embodiments, some or all of the PNA residues are miniPEG-containing γPNAs (Sahu, et al., J. Org. Chem., 76, 5614-5627 (2011). In some embodiments, tcPNAs are prepared wherein every other PNA residue on the Watson-Crick binding side of the linker is a miniPEG-containing γPNA. Accordingly, for these embodiments, the tail clamp side of the PNA has alternating classic PNA and miniPEG-containing γPNA residues.

In some embodiments PNA-mediated gene editing are achieved via additional or alternative γ substitutions or other PNA chemical modifications including but limited to those introduced above and below. Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In addition to γPNAs showing consistently improved gene editing potency the level of off-target effects in the genome remains extremely low. This is in keeping with the lack of any intrinsic nuclease activity in the PNAs (in contrast to ZFNs or CRISPR/Cas9 or TALENS), and reflects the mechanism of triplex-induced gene editing, which acts by creating an altered helix at the target-binding site that engages endogenous high fidelity DNA repair pathways. As discussed above, the SCF/c-Kit pathway also stimulates these same pathways, providing for enhanced gene editing without increasing off-target risk or cellular toxicity.

Additionally, any of the triplex-forming sequences can be modified to include guanidine-G-clamp (“G-clamp”) PNA residues(s) to enhance PNA binding, wherein the G-clamp is linked to the backbone as any other nucleobase would be. γPNAs with substitution of cytosine by clamp-G (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single clamp-G substitution for C can substantially enhance the binding of a PNA-DNA duplex by 23° C. (Kuhn, et al., Artificial DNA, PNA & XNA, 1(1):45-53(2010)). As a result, γPNAs containing G-clamp substitutions can have further increased activity.

The structure of a clamp-G monomer-to-G base pair (clamp-G indicated by the “X”) is illustrated below in comparison to C-G base pair.

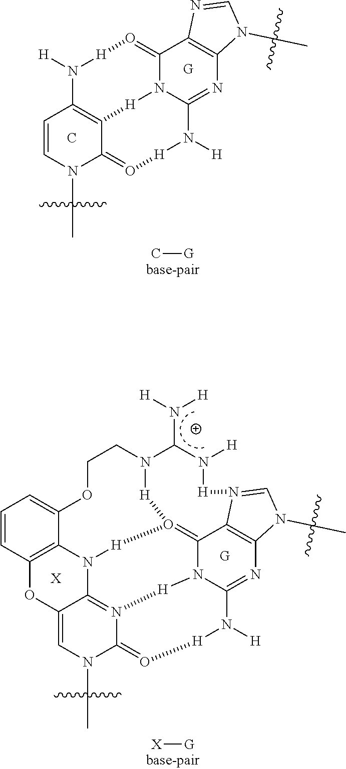

Some studies have shown improvements using D-amino acids in peptide synthesis.

In particular embodiments, the gene editing composition includes at least one peptide nucleic acid (PNA) oligomer. The at least one PNA oligomer can be a modified PNA oligomer including at least one modification at a gamma position of a backbone carbon. The modified PNA oligomer can include at least one miniPEG modification at a gamma position of a backbone carbon. The gene editing composition can include at least one donor oligonucleotide. The gene editing composition can modify a target sequence within a fetal genome.

The PNA can include a Hoogsteen binding peptide nucleic acid (PNA) segment and a Watson-Crick binding PNA segment collectively totaling no more than 50 nucleobases in length, wherein the two segments bind or hybridize to a target region of a genomic DNA comprising a polypurine stretch to induce strand invasion, displacement, and formation of a triple-stranded composition among the two PNA segments and the polypurine stretch of the genomic DNA, wherein the Hoogsteen binding segment binds to the target region by Hoogsteen binding for a length of least five nucleobases, and wherein the Watson-Crick binding segment binds to the target region by Watson-Crick binding for a length of least five nucleobases.

The PNA segments can include a gamma modification of a backbone carbon. The gamma modification can be a gamma miniPEG modification. The Hoogsteen binding segment can include one or more chemically modified cytosines selected from the group consisting of pseudocytosine, pseudoisocytosine, and 5-methylcytosine. The Watson-Crick binding segment can include a sequence of up to fifteen nucleobases that binds to the target duplex by Watson-Crick binding outside of the triplex. The two segments can be linked by a linker. In some embodiments, all of the peptide nucleic acid residues in the Hoogsteen-binding segment only, in the Watson-Crick-binding segment only, or across the entire PNA oligomer include a gamma modification of a backbone carbon. In some embodiments, one or more of the peptide nucleic acid residues in the Hoogsteen-binding segment only or in the Watson-Crick-binding segment only of the PNA oligomer include a gamma modification of a backbone carbon. In some embodiments, alternating peptide nucleic acid residues in the Hoogsteen-binding portion only, in the Watson-Crick-binding portion only, or across the entire PNA oligomer include a gamma modification of a backbone carbon.

In some embodiments, least one gamma modification of the backbone carbon is a gamma miniPEG modification. In some embodiments, at least one gamma modification is a side chain of an amino acid selected from the group consisting of alanine, serine, threonine, cysteine, valine, leucine, isoleucine, methionine, phenylalanine, tyrosine, aspartic acid, glutamic acid, asparagine, glutamine, histidine, lysine, arginine, and the derivatives thereof. In some embodiments, all gamma modifications are gamma miniPEG modifications. Optionally, at least one PNA segment comprises a clamp-G (9-(2-guanidinoethoxy) phenoxazine).

2. Triplex-Forming Target Sequence Considerations

The triplex-forming molecules bind to a predetermined target region referred to herein as the “target sequence,” “target region,” or “target site.” The target sequence for the triplex-forming molecules can be within or adjacent to a human gene encoding, for example the beta globin, cystic fibrosis transmembrane conductance regulator (CFTR) or other gene discussed in more detail below, or an enzyme necessary for the metabolism of lipids, glycoproteins, or mucopolysaccharides, or another gene in need of correction. The target sequence can be within the coding DNA sequence of the gene or within an intron. The target sequence can also be within DNA sequences which regulate expression of the target gene, including promoter or enhancer sequences or sites that regulate RNA splicing.

The nucleotide sequences of the triplex-forming molecules are selected based on the sequence of the target sequence, the physical constraints, and the preference for a low dissociation constant (K_{d}) for the triplex-forming molecules/target sequence. As used herein, triplex-forming molecules are said to be substantially complementary to a target region when the triplex-forming molecules has a nucleobase composition which allows for the formation of a triple-helix with the target region. A triplex-forming molecule can be substantially complementary to a target region even when there are non-complementary nucleobases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

a. Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

TFOs are preferably generated using known DNA and/or PNA synthesis procedures. In one embodiment, oligonucleotides are generated synthetically. Oligonucleotides can also be chemically modified using standard methods that are well known in the art.

b. Target Sequence Considerations for PNAs

Some triplex-forming molecules, such as PNA, PNA clamps and tail clamp PNAs (tcPNAs) invade the target duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex-forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleobase-containing residues in length. The Watson-Crick portion should be 9 or more nucleobase-containing residues in length, optionally including a tail sequence. More preferably, the Watson-Crick binding portion is between about 9 and 30 nucleobase-containing residues in length, optionally including a tail sequence of between 0 and about 15 nucleobase-containing residues. More preferably, the Watson-Crick binding portion is between about 10 and 25 nucleobase-containing residues in length, optionally including a tail sequence of between 0 and about 10 nucleobase-containing residues in length. In the most preferred embodiment, the Watson-Crick binding portion is between 15 and 25 nucleobase-containing residues in length, optionally including a tail sequence of between 5 and 10 nucleobase-containing residues in length. The Hoogsteen binding portion should be 6 or more nucleobase residues in length. Most preferably, the Hoogsteen binding portion is between about 6 and 15 nucleobase-containing residues in length, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobase-containing residues, known as a “tail,” to the Watson-Crick binding portion of the triplex-forming molecules allows the Watson-Crick binding portion to bind/hybridize to the target strand outside the site of polypurine sequence for triplex formation. These additional bases further reduce the requirement for the polypurine:polypyrimidine stretch in the target duplex and therefore increase the number of potential target sites. Triplex-forming molecules (TFMs) including, e.g., triplex-forming oligonucleotides (TFOs) and helix-invading peptide nucleic acids (bis-PNAs and tcPNAs), also generally utilize a polypurine:polypyrimidine sequence to a form a triple helix. Traditional nucleic acid TFOs may need a stretch of at least 15 and preferably 30 or more nucleobase-containing residues. Peptide nucleic acids need fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. In one embodiment, triplex-forming molecules are generated synthetically. Triplex-forming molecules can also be chemically modified using standard methods that are well known in the art.

B. Pseudocomplementary Oligonucleotides/PNAs

The gene editing technology can be pseudocomplementary oligonucleotides such as those disclosed in U.S. Pat. No. 8,309,356. “Double duplex-forming molecules,” are oligonucleotides that bind to duplex DNA in a sequence-specific manner to form a four-stranded structure. Double duplex-forming molecules, such as a pair of pseudocomplementary oligonucleotides/PNAs, can induce recombination with a donor oligonucleotide at a chromosomal site in mammalian cells. Pseudocomplementary oligonucleotides/PNAs are complementary oligonucleotides/PNAs that contain one or more modifications such that they do not recognize or hybridize to each other, for example due to steric hindrance, but each can recognize and hybridize to its complementary nucleic acid strands at the target site. As used herein the term ‘pseudocomplementary oligonucleotide(s)’ include pseudocomplementary peptide nucleic acids (pcPNAs). A pseudocomplementary oligonucleotide is said to be substantially complementary to a target region when the oligonucleotide has a base composition which allows for the formation of a double duplex with the target region. As such, an oligonucleotide can be substantially complementary to a target region even when there are non-complementary bases present in the pseudocomplementary oligonucleotide.

This strategy can be more efficient and provides increased flexibility over other methods of induced recombination such as triple-helix oligonucleotides and bis-peptide nucleic acids which prefer a polypurine sequence in the target double-stranded DNA. The design ensures that the pseudocomplementary oligonucleotides do not pair with each other but instead bind the cognate nucleic acids at the target site, inducing the formation of a double duplex.

The predetermined region that the double duplex-forming molecules bind to can be referred to as a “double duplex target sequence,” “double duplex target region,” or “double duplex target site.” The double duplex target sequence (DDTS) for the double duplex-forming molecules can be, for example, within or adjacent to a human gene in need of induced gene correction. The DDTS can be within the coding DNA sequence of the gene or within introns. The DDTS can also be within DNA sequences which regulate expression of the target gene, including promoter or enhancer sequences.

The nucleotide/nucleobase sequence of the pseudocomplementary oligonucleotides is selected based on the sequence of the DDTS. Therapeutic administration of pseudocomplementary oligonucleotides involves two single stranded oligonucleotides unlinked, or linked by a linker. One pseudocomplementary oligonucleotide strand is complementary to the DDTS, while the other is complementary to the displaced DNA strand. The use of pseudocomplementary oligonucleotides, particularly pcPNAs are not subject to limitation on sequence choice and/or target length and specificity as are triplex-forming oligonucleotides, helix-invading peptide nucleic acids (bis-PNAs and tcPNAs) and side-by-side minor groove binders. Pseudocomplementary oligonucleotides do not require third-strand Hoogsteen-binding, and therefore are not restricted to homopurine targets. Pseudocomplementary oligonucleotides can be designed for mixed, general sequence recognition of a desired target site. Preferably, the target site contains an A:T base pair content of about 40% or greater. Preferably pseudocomplementary oligonucleotides are between about 8 and 50 nucleobase-containing residues in length, more preferably 8 to 30, even more preferably between about 8 and 20 nucleobase-containing residues in length.

The pseudocomplementary oligonucleotides should be designed to bind to the target site (DDTS) at a distance of between about 1 to 800 bases from the target site of the donor oligonucleotide. More preferably, the pseudocomplementary oligonucleotides bind at a distance of between about 25 and 75 bases from the donor oligonucleotide. Most preferably, the pseudocomplementary oligonucleotides bind at a distance of about 50 bases from the donor oligonucleotide. Preferred pcPNA sequences for targeted repair of a mutation in the β-globin intron IVS2 (G to A) are described in U.S. Pat. No. 8,309,356.

Preferably, the pseudocomplementary oligonucleotides bind/hybridize to the target nucleic acid molecule under conditions of high stringency and specificity. Most preferably, the oligonucleotides bind in a sequence-specific manner and induce the formation of double duplex. Specificity and binding affinity of the pseudocomplemetary oligonucleotides may vary from oligomer to oligomer, depending on factors such as length, the number of G:C and A:T base pairs, and the formulation.

### V. Donor Oligonucleotides

In some embodiments, the gene editing composition includes or is administered in combination with a donor oligonucleotide. The donor oligonucleotide can be encapsulated or entrapped in the same or different particles from other active agents such as the triplex-forming composition. Generally, in the case of gene therapy, the donor oligonucleotide includes a sequence that can correct a mutation(s) in the host genome, though in some embodiments, the donor introduces a mutation that can, for example, reduce expression of an oncogene or a receptor that facilitates HIV infection. In addition to containing a sequence designed to introduce the desired correction or mutation, the donor oligonucleotide may also contain synonymous (silent) mutations (e.g., 7 to 10). The additional silent mutations can facilitate detection of the corrected target sequence using allele-specific PCR of genomic DNA isolated from treated cells. Triplex-forming composition and other gene editing compositions such as those discussed above can increase the rate of recombination of the donor oligonucleotide in the target cells relative to administering donor alone.

The triplex-forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence targets a region between 0 to 800 bases from the target binding site of the triplex-forming molecules. More preferably the donor oligonucleotide sequence targets a region between 25 to 75 bases from the target binding site of the triplex-forming molecules. Most preferably that the donor oligonucleotide sequence targets a region about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to herein as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex-forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the oligonucleotide donor is between 25 and 80 nucleobases. In a further embodiment, the non-tethered donor oligonucleotide is about 50 to 60 nucleotides in length.

The donor oligonucleotides contain at least one mutated, inserted or deleted nucleotide relative to the target DNA sequence. Target sequences can be within the coding DNA sequence of the gene or within introns. Target sequences can also be within DNA sequences which regulate expression of the target gene, including promoter or enhancer sequences or sequences that regulate RNA splicing.

The donor oligonucleotides can contain a variety of mutations relative to the target sequence. Representative types of mutations include, but are not limited to, point mutations, deletions and insertions. Deletions and insertions can result in frameshift mutations or deletions. Point mutations can cause missense or nonsense mutations. These mutations may disrupt, reduce, stop, increase, improve, or otherwise alter the expression of the target gene.

Compositions including triplex-forming molecules such as tcPNA may include one or more than one donor oligonucleotides. More than one donor oligonucleotides may be administered with triplex-forming molecules in a single transfection, or sequential transfections. Use of more than one donor oligonucleotide may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic nucleobases, deoxyribose as the sugar moiety, and phosphate ester linkages. Donor oligonucleotides may include modifications to nucleobases, sugar moieties, or backbone/linkages, as described above, depending on the desired structure of the replacement sequence at the site of recombination or to provide some resistance to degradation by nucleases. One exemplary modification is a thiophosphate ester linkage. Modifications to the donor oligonucleotide should not prevent the donor oligonucleotide from successfully recombining at the recombination target sequence in the presence of triplex-forming molecules.

### VI. Nucleobase, Sugar, and Linkage Modifications

Any of the gene editing technologies, components thereof, donor oligonucleotides, or other nucleic acids disclosed herein can include one or more modifications or substitutions to the nucleobases or linkages. Although modifications are particularly preferred for use with triplex-forming technologies and typically discussed below with reference thereto, any of the modifications can be utilized in the construction of any of the gene editing compositions, donor, nucleotides, etc. Modifications should not prevent, and preferably enhance the activity, persistence, or function of the gene editing technology. For example, modifications to oligonucleotides for use as triplex-forming molecules should not prevent, and preferably enhance duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in the molecules disclosed herein. Several preferred oligonucleotide compositions including PNA, and modification thereof to include MiniPEG at the γ position in the PNA backbone, are discussed above. Additional modifications are discussed in more detail below.

A. Nucleobases

The principal naturally-occurring nucleotides include uracil, thymine, cytosine, adenine and guanine as the heterocyclic nucleobases. Gene editing molecules can include chemical modifications to their nucleotide constituents. For example, target sequences with adjacent cytosines can be problematic. Triplex stability is greatly compromised by runs of cytosines, thought to be due to repulsion between the positive charge resulting from the N³ protonation or perhaps because of competition for protons by the adjacent cytosines. Chemical modification of nucleotides including triplex-forming molecules such as PNAs may be useful to increase binding affinity of triplex-forming molecules and/or triplex stability under physiologic conditions.

Chemical modifications of nucleobases or nucleobase analogs may be effective to increase the binding affinity of a nucleotide or its stability in a triplex. Chemically-modified nucleobases include, but are not limited to, inosine, 5-(1-propynyl) uracil (pU), 2-thio uracil, 5-(1-propynyl) cytosine (pC), 5-methylcytosine, 8-oxo-adenine, 2,6-diaminopurine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines.

B. Backbone

The nucleotide residues of the triplex-forming molecules such as PNAs are connected by an internucleotide bond that refers to a chemical linkage between two nucleoside moieties. Unmodified peptide nucleic acids (PNAs) are synthetic DNA mimics in which the phosphate backbone of the oligonucleotide is replaced in its entirety by repeating N-(2-aminoethyl)-glycine units that are linked by amide bonds. The various nucleobases are linked to the backbone by methylene carbonyl bonds, which allow them to form PNA-DNA or PNA-RNA duplexes via Watson-Crick base pairing with high affinity and sequence-specificity. PNAs maintain spacing of nucleobases that is similar to conventional DNA oligonucleotides, but are achiral and neutrally charged molecules. Peptide nucleic acids are composed of peptide nucleic acid residues.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications used to generate triplex-forming molecules should not prevent the molecules from binding with high specificity to the target site and creating a triplex with the target duplex nucleic acid by displacing one strand of the target duplex and forming a clamp around the other strand of the target duplex.

C. Modified Nucleic Acids

Modified nucleic acids in addition to peptide nucleic acids are also useful as triplex-forming molecules. Oligonucleotides are composed a chain of nucleotides which are linked to one another. Canonical nucleotides typically are composed of a nucleobase (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic nucleobases, and ribose or deoxyribose sugar linked by phosphodiester bonds. As used herein “modified nucleotide” or “chemically modified nucleotide” defines a nucleotide that has a chemical modification of one or more of the nucleobase, sugar moiety or phosphate moiety constituents. Preferably the charge of the modified nucleotide is reduced compared to DNA or RNA oligonucleotides of the same nucleobase sequence. Most preferably the triplex-forming molecules have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be except they have a negatively charged backbone, whereas PNAs generally have a neutrally charged backbone (although certain amino acid side chain modifications can alter the backbone charge). LNA binding efficiency can be increased in some embodiments by adding positive charges to it. Commercial nucleic acid synthesizers and standard phosphoramidite chemistry can be used to make LNAs.

Molecules may also include nucleotides with modified nucleobases, sugar moieties or sugar moiety analogs. Modified nucleotides may include modified nucleobases or base analogs as described above with respect to peptide nucleic acids. Sugar moiety modifications include, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O—(N-(methyl)acetamido) (2′-OMA). 2′-O-aminoethyl sugar moiety substitutions are especially preferred because they are protonated at neutral pH and thus suppress the charge repulsion between the triplex-forming molecule and the target duplex. This modification stabilizes the C3′-endo conformation of the ribose or deoxyribose and also forms a bridge with the i-1 phosphate in the purine strand of the duplex.

### VII. Gene Editing Potentiating Factors

In some embodiments, the compositions and methods include a potentiating factor. For example, certain potentiating factors can be used to increase the efficacy of gene editing technologies. Gene expression profiling on SCF-treated CD117+ cells versus untreated CD117+ cells discussed in the Examples below showed additional up-regulation of numerous DNA repair genes including RAD51 and BRCA2. These results and others discussed below indicate that a functional c-Kit signaling pathway mediates increased HDR and promotes gene editing, rather than CD117 simply being a phenotypic marker. When CD117+ cells were treated with SCF, expression of these DNA repair genes was increased even more, correlating with a further increase in gene editing.

Accordingly, compositions and methods of increasing the efficacy of gene editing technology are provided. As used herein a “gene editing potentiating factor” or “gene editing potentiating agent” or “potentiating factor or “potentiating agent” refers a compound that increases the efficacy of editing (e.g., mutation, including insertion, deletion, substitution, etc.) of a gene, genome, or other nucleic acid) by a gene editing technology relative to use of the gene editing technology in the absence of the compound. Preferred gene editing technologies suitable for use alone or more preferably in combination with the potentiating factors are discussed in more detail below. In some embodiments, the potentiating factor is administered as a nucleic acid encoding the potentiating factor. In certain preferred embodiments, the gene editing technology is a triplex-forming γPNA and donor DNA, optionally, but preferably in a particle composition.

Potentiating factors include, for example, DNA damage or repair-stimulating or -potentiating factors. Preferably the factor is one that engages one or more endogenous high fidelity DNA repair pathways. In some embodiments, the factor is one that increases expression of Rad51, BRCA2, or a combination thereof.

As discussed in more detail below, the preferred methods typically include contacting cells with an effective amount of a gene editing potentiating factor. The contacting can occur ex vivo, for example isolated cells, or in vivo following, for example, administration of the potentiating factor to a subject. Examplary gene editing potentiating agents include receptor tyrosine kinase C-kit ligands, ATR-Chk1 cell cycle checkpoint pathway inhibitors, a DNA polymerase alpha inhibitors, and heat shock protein 90 inhibitors (HSP90i).

A. C-Kit Ligands

In some embodiments, the factor is an activator of the receptor tyrosine kinase c-Kit. CD117 (also known as mast/stem cell growth factor receptor or proto-oncogene c-Kit protein) is a receptor tyrosine kinase expressed on the surface of hematopoietic stem and progenitor cells as well as other cell types. Stem cell factor (SCF), the ligand for c-Kit, causes dimerization of the receptor and activates its tyrosine kinase activity to trigger downstream signaling pathways that can impact survival, proliferation, and differentiation. SCF and c-Kit are reviewed in Lennartsson and Ronnstrand, Physiological Reviews, 92(4):1619-1649 (2012)). Thus, in some embodiments, the C-kit ligand is stem factor protein or fragment thereof sufficient to causes dimerization of C-kit and activates its tyrosine kinase activity. The C-kit ligand can be a nucleic acid encoding a stem factor protein or fragment thereof sufficient to causes dimerization of C-kit and activates its tyrosine kinase activity. The nucleic acid can be an mRNA or an expression vector.

The human SCF gene encodes for a 273 amino acid transmembrane protein, which contains a 25 amino acid N-terminal signal sequence, a 189 amino acid extracellular domain, a 23 amino acid transmembrane domain, and a 36 amino acid cytoplasmic domain. A canonical human SCF amino acid sequence is:

(SEQ ID NO: 1, UniProtKB - P21583 (SCF_HUMAN))
MKKTQTWILTCIYLQLLLFNPLVKTEGICRNRVTNNVKDVTKLVANL
PKDYMITLKYVPGMDVLPSHCWISEMVVQLSDSLTDLLDKFSNISEG
LSNYSIIDKLVNIVDDLVECVKENSSKDLKKSFKSPEPRLFTPEEFFRIF
NRSIDAFKDFVVASETSDCVVSSTLSPEKDSRVSVTKPFMLPPVAASS
LRNDSSSSNRKAKNPPGDSSLHWAAMALPALFSLIIGFAFGALYWKK
RQPSLTRAVENIQINEEDNEISMLQEKEREFQEV.

The secreted soluble form of SCF is generated by proteolytic processing of the membrane-anchored precursor. A cleaved, secreted soluble form of human SCF is underlined in SEQ ID NO:1, which corresponds to SEQ ID NO:2 without the N-terminal methionine.

(SEQ ID NO: 2, Preprotech Recombinant Human
SCF Catalog Number: 300-07)
MEGICRNRVTNNVKDVTKLVANLPKDYMITLKYVPGMDVLPSHCWI
SEMVVQLSDSLTDLLDKFSNISEGLSNYSIIDKLVNIVDDLVECVKEN
SSKDLKKSFKSPEPRLFTPEEFFRIFNRSIDAFKDFVVASETSDCVVSST
LSPEKD SRVSVTKPFMLPPVA.

Murine and rat SCF are fully active on human cells. A canonical mouse SCF amino acid sequence is:

(SEQ ID NO: 3, UniProtKB - P20826 (SCF_MOUSE))
MKKTQTWIITCIYLQLLLFNPLVKTKEICGNPVTDNVKDITKLVANLP
NDYMITLNYVAGMDVLPSHCWLRDMVIQLSLSLTTLLDKFSNISEGL
SNYSIIDKLGKIVDDLVLCMEENAPKNIKESPKRPETRSFTPEEFFSIFN
RSIDAFKDFMVASDTSDCVLSSTLGPEKDSRVSVTKPFMLPPVAASSL
RNDSSSSNRKAAKAPEDSGLQWTAMALPALISLVIGFAFGALYWKK
KQSSLTRAVENIQINEEDNEISMLQQKEREFQEV.

A cleaved, secreted soluble form of mouse SCF is underlined in SEQ ID NO:3, which corresponds to SEQ ID NO:4 without the N-terminal methionine.

(SEQ ID NO: 4, Preprotech Recombinant
Murine SCF Catalog Number: 250-03)
MKEICGNPVTDNVKDITKLVANLPNDYMITLNYVAGMDVLPSHCWL
RDMVIQLSLSLTTLLDKFSNISEGLSNYSIIDKLGKIVDDLVLCMEENA
PKNIKESPKRPETRSFTPEEFFSIFNRSIDAFKDFMVASDTSDCVLSSTL
GPEKDSRVSVTKPFMLPPVA

A canonical mouse SCF amino acid sequence is:

(SEQ ID NO: 5, UniProtKB - P21581 (SCF_RAT))
MKKTQTWIITCIYLQLLLFNPLVKTQEICRNPVTDNVKDITKLVANLP
NDYMITLNYVAGMDVLPSHCWLRDMVTHLSVSLTTLLDKFSNISEG
LSNYSIIDKLGKIVDDLVACMEENAPKNVKESLKKPETRNFTPEEFFSI
FNRSIDAFKDFMVASDTSDCVLSSTLGPEKDSRVSVTKPFMLPPVAAS
SLRNDSSSSNRKAAKSPEDPGLQWTAMALPALISLVIGFAFGALYWK
KKQSSLTRAVENIQINEEDNEISMLQQKEREFQEV.

A cleaved, secreted soluble form of rat SCF is underlined in SEQ ID NO:5, which corresponds to SEQ ID NO:6 without the N-terminal methionine.

MQEICRNPVTDNVKDITKLVANLPNDYMITLNYVAGMDVLPSHCWL RDMVTHLSVSLTTLLDKFSNISEGLSNYSIIDKLGKIVDDLVACMEEN APKNVKESLKKPETRNFTPEEFFSIFNRSIDAFKDFMVASDTSDCVLSS TLGPEKDSRVSVTKPFMLPPVA (SEQ ID NO:6, Shenandoah Biotechnology, Inc., Recombinant Rat SCF (Stem Cell Factor) Catalog Number: 300-32).

In some embodiments, the factor is a SCF such as any of SEQ ID NO:1-6, with or without the N-terminal methionine, or a functional fragment thereof, or a variant thereof with at least 60, 65, 70, 75, 80, 85, 90, 95, 96, 97, 98, 99, or more sequence identity to any one of SEQ ID NO:1-6.

It will be appreciated that SCF can be administered to cells or a subject as SCF protein, or as a nucleic acid encoding SCF (transcribed RNA, DNA, DNA in an expression vector). Accordingly, nucleic acid sequences, including RNA (e.g., mRNA) and DNA sequences, encoding SEQ ID NOS:1-6 are also provided, both alone and inserted into expression cassettes and vectors. For example, a sequence encoding SCF can be incorporated into an autonomously replicating plasmid, a virus (e.g., a retrovirus, lentivirus, adenovirus, or herpes virus), or into the genomic DNA of a prokaryote or eukaryote.

The observed effect of SCF indicates that other cytokines or growth factors including, but not limited to, erythropoietin, GM-CSF, EGF (especially for epithelial cells; lung epithelia for cystic fibrosis), hepatocyte growth factor etc., could similarly serve to boost gene editing potential in bone marrow cells or in other tissues. In some embodiments, gene editing is enhanced in specific cell types using cytokines targeted to these cell types.

B. Replication Modulators

In some embodiments, the potentiating factor is a replication modulator that can, for example, manipulate replication progression and/or replication forks. For example, the ATR-Chk1 cell cycle checkpoint pathway has numerous roles in protecting cells from DNA damage and stalled replication, one of the most prominent being control of the cell cycle and prevention of premature entry into mitosis (Thompson and Eastman, Br J Clin Pharmacol., 76(3): 358-369 (2013), Smith, et al., Adv Cancer Res., 108:73-112 (2010)). However, Chk1 also contributes to the stabilization of stalled replication forks, the control of replication origin firing and replication fork progression, and homologous recombination. DNA polymerase alpha also known as Pol α is an enzyme complex found in eukaryotes that is involved in initiation of DNA replication. Hsp90 (heat shock protein 90) is a chaperone protein that assists other proteins to fold properly, stabilizes proteins against heat stress, and aids in protein degradation.

Inhibitors of CHK1 and ATR in the DNA damage response pathway, as well as DNA polymerase alpha inhibitors and HSP90 inhibitors, substantially boost gene editing by triplex-forming PNAs and single-stranded donor DNA oligonucleotides. Accordingly, in some embodiments, the potentiating factor is a CHK1 or ATR pathway inhibitor, a DNA polymerase alpha inhibitor, or an HSP90 inhibitor. The inhibitor can be a functional nucleic acid, for example siRNA, miRNA, aptamers, ribozymes, triplex-forming molecules, RNAi, or external guide sequences that targets CHK1, ATR, or another molecule in the ATR-Chk1 cell cycle checkpoint pathway; DNA polymerase alpha; or HSP90 and reduces expression or active of ATR, CHK1, DNA polymerase alpha, or HSP90.

Preferably, the inhibitor is a small molecule. For example, the potentiating factor can be a small molecule inhibitor of ATR-Chk1 Cell Cycle Checkpoint Pathway Inhibitor. Such inhibitors are known in the art, and many have been tested in clinical trials for the treatment of cancer. Exemplary CHK1 inhibitors include, but are not limited to, AZD7762, SCH900776/MK-8776, IC83/LY2603618, LY2606368, GDC-0425, PF-00477736, XL844, CEP-3891, SAR-020106, CCT-244747, Arry-575 (Thompson and Eastman, Br J Clin Pharmacol., 76(3): 358-369 (2013)), and SB218075. Exemplary ATR pathway inhibitors include, but are not limited to Schisandrin B, NU6027, NVP-BEZ235, VE-821, VE-822 (VX-970), AZ20, AZD6738, MIRIN, KU5593, VE-821, NU7441, LCA, and L189 (Weber and Ryan, Pharmacology & Therapeutics, 149:124-138 (2015)).

In some embodiments, the potentiating factor is a DNA polymerase alpha inhibitor, such as aphidicolin.

In some embodiments, the potentiating factor is a heat shock protein 90 inhibitor (HSP90i) such as STA-9090 (ganetespib). Other HSP90 inhibitors are known in the art and include, but are not limited to, benzoquinone ansamycin antibiotics such as geldanamycin (GA); 17-AAG (17-Allylamino-17-demethoxy-geldanamycin); 17-DMAG (17-dimethylaminoethylamino-17-demethoxy-geldanamycin) (Alvespimycin); IPI-504 (Retaspimycin); and AUY922 (Tatokoro, et al., EXCLI J., 14:48-58 (2015)).

### VIII. Functional Molecules

Functional molecules can be associated with, linked, conjugated, or otherwise attached directly or indirectly gene editing technology, potentiating agents, or particles utilized for delivery thereof. For example, the composition can include a targeting agent, a cell penetrating peptide, or a combination thereof. In some embodiments, two or more targeting molecules are used. The targeting agent is typically capable of specifically binding to a target on or in the embryo. Target agents can be bound or conjugated to particles (e.g., a polymer of the particle).

A. Targeting Molecules

One class of functional elements is targeting molecules. Targeting molecules can be associated with, linked, conjugated, or otherwise attached directly or indirectly to the gene editing molecule, or to a particle or other delivery vehicle thereof.

Targeting molecules can be proteins, peptides, nucleic acid molecules, saccharides or polysaccharides that bind to a receptor or other molecule on the surface of a targeted cell. The degree of specificity and the avidity of binding to the graft can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34⁺ cells, T cells or any other preferred cell type, as well as receptor and ligands expressed on the preferred cell type. Preferably, the moieties target hematopoeitic stem cells.

In some embodiments, the targeting molecule targets a cell surface protein.

Examples of molecules targeting extracellular matrix (“ECM”) include glycosaminoglycan (“GAG”) and collagen. In one embodiment, the external surface of polymer particles may be modified to enhance the ability of the particles to interact with selected cells or tissue. The method described above wherein an adaptor element conjugated to a targeting molecule is inserted into the particle is preferred. However, in another embodiment, the outer surface of a polymer micro- or nanoparticle having a carboxy terminus may be linked to targeting molecules that have a free amine terminus.

Other useful ligands attached to polymeric micro- and nanoparticles include pathogen-associated molecular patterns (PAMPs). PAMPs target Toll-like Receptors (TLRs) on the surface of the cells or tissue, or signal the cells or tissue internally, thereby potentially increasing uptake. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the particle may be treated using a mannose amine, thereby mannosylating the outer surface of the particle. This treatment may cause the particle to bind to the target cell or tissue at a mannose receptor on the antigen presenting cell surface. Alternatively, surface conjugation with an immunoglobulin molecule containing an Fc portion (targeting Fc receptor), heat shock protein moiety (HSP receptor), phosphatidylserine (scavenger receptors), and lipopolysaccharide (LPS) are additional receptor targets on cells or tissue.

Lectins that can be covalently attached to micro- and nanoparticles to render them target specific to the mucin and mucosal cell layer include lectins isolated from Abrus precatroius, Agaricus bisporus, Anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The choice of targeting molecule will depend on the method of administration of the particle composition and the cells or tissues to be targeted. The targeting molecule may generally increase the binding affinity of the particles for cell or tissues or may target the particle to a particular tissue in an organ or a particular cell type in a tissue. Avidin increases the ability of polymeric particles to bind to tissues. While the exact mechanism of the enhanced binding of avidin-coated particles to tissues has not been elucidated, it is hypothesized it is caused by electrostatic attraction of positively charged avidin to the negatively charged extracellular matrix of tissue. Non-specific binding of avidin, due to electrostatic interactions, has been previously documented and zeta potential measurements of avidin-coated PLGA particles revealed a positively charged surface as compared to uncoated PLGA particles.

The attachment of any positively charged ligand, such as polyethyleneimine or polylysine, to any polymeric particle may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most particles with the appropriate chemistry and be expected to influence the binding of particles to the gut. For example, polyclonal antibodies raised against components of mucin or else intact mucin, when covalently coupled to particles, would provide for increased bioadhesion. Similarly, antibodies directed against specific cell surface receptors exposed on the lumenal surface of the intestinal tract would increase the residence time of beads, when coupled to particles using the appropriate chemistry. The ligand affinity need not be based only on electrostatic charge, but other useful physical parameters such as solubility in mucin or else specific affinity to carbohydrate groups.

The covalent attachment of any of the natural components of mucin in either pure or partially purified form to the particles would decrease the surface tension of the bead-gut interface and increase the solubility of the bead in the mucin layer. The list of useful ligands includes, but is not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range yields chains of 120 to 425 amino acid residues attached to the surface of the particles. The polyamino chains increase bioadhesion by means of chain entanglement in mucin strands as well as by increased carboxylic charge.

The efficacy of the particles is determined in part by their route of administration into the body. For orally and topically administered particles, epithelial cells constitute the principal barrier that separates an organism's interior from the outside world. Epithelial cells such as those that line the gastrointestinal tract form continuous monolayers that simultaneously confront the extracellular fluid compartment and the extracorporeal space.

Adherence to cells is an essential first step in crossing the epithelial barrier by any of these mechanisms. Therefore, in one embodiment, the particles herein further include epithelial cell targeting molecules. Epithelial cell targeting molecules include monoclonal or polyclonal antibodies or bioactive fragments thereof that recognize and bind to epitopes displayed on the surface of epithelial cells. Epithelial cell targeting molecules also include ligands which bind to a cell surface receptor on epithelial cells. Ligands include, but are not limited to, molecules such as polypeptides, nucleotides and polysaccharides.

A variety of receptors on epithelial cells may be targeted by epithelial cell targeting molecules. Examples of suitable receptors to be targeted include, but are not limited to, IgE Fc receptors, EpCAM, selected carbohydrate specificites, dipeptidyl peptidase, and E-cadherin.

B. Protein Transduction Domains and Fusogenic Peptides

Other functional elements that can be associated with, linked, conjugated, or otherwise attached directly or indirectly to the gene editing molecule, potentiating agent, or to a particle or other delivery vehicle thereof, include protein transduction domains and fusogenic peptides.

For example, the efficiency of particle delivery systems can also be improved by the attachment of functional ligands to the particle surface. Potential ligands include, but are not limited to, small molecules, cell-penetrating peptides (CPPs), targeting peptides, antibodies or aptamers (Yu, et al., PLoS One., 6:e24077 (2011), Cu, et al., J Control Release, 156:258-264 (2011), Nie, et al., J Control Release, 138:64-70 (2009), Cruz, et al., J Control Release, 144:118-126 (2010)). Attachment of these moieties serves a variety of different functions; such as inducing intracellular uptake, endosome disruption, and delivery of the plasmid payload to the nucleus. There have been numerous methods employed to tether ligands to the particle surface. One approach is direct covalent attachment to the functional groups on PLGA NPs (Bertram, Acta Biomater. 5:2860-2871 (2009)). Another approach utilizes amphiphilic conjugates like avidin palmitate to secure biotinylated ligands to the NP surface (Fahmy, et al., Biomaterials, 26:5727-5736 (2005), Cu, et al., Nanomedicine, 6:334-343 (2010)). This approach produces particles with enhanced uptake into cells, but reduced pDNA release and gene transfection, which is likely due to the surface modification occluding pDNA release. In a similar approach, lipid-conjugated polyethylene glycol (PEG) is used as a multivalent linker of penetratin, a CPP, or folate (Cheng, et al., Biomaterials, 32:6194-6203 (2011)).

These methods, as well as other methods discussed herein, and others methods known in the art, can be combined to tune particle function and efficacy. In some preferred embodiments, PEG is used as a linker for linking functional molecules to particles. For example, DSPE-PEG(2000)-maleimide is commercially available and can be used utilized for covalently attaching functional molecules such as CPP.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. PTA can be short basic peptide sequences such as those present in many cellular and viral proteins. Exemplary protein transduction domains that are well-known in the art include, but are not limited to, the Antennapedia PTD and the TAT (transactivator of transcription) PTD, poly-arginine, poly-lysine or mixtures of arginine and lysine, HIV TAT (YGRKKRRQRRR (SEQ ID NO:7) or RKKRRQRRR (SEQ ID NO:8), 11 arginine residues, VP22 peptide, and an ANTp peptide (RQIKIWFQNRRMKWKK) (SEQ ID NO:9) or positively charged polypeptides or polynucleotides having 8-15 residues, preferably 9-11 residues. Short, non-peptide polymers that are rich in amines or guanidinium groups are also capable of carrying molecules crossing biological membranes. Penetratin and other derivatives of peptides derived from antennapedia (Cheng, et al., Biomaterials, 32(26):6194-203 (2011) can also be used. Results show that penetratin in which additional Args are added, further enhances uptake and endosomal escape, and IKK NBD, which has an antennapedia domain for permeation as well as a domain that blocks activation of NFkB and has been used safely in the lung for other purposes (von Bismarck, et al., Pulmonary Pharmacology & Therapeutics, 25(3):228-35 (2012), Kamei, et al., Journal Of Pharmaceutical Sciences, 102(11):3998-4008 (2013)).

A “fusogenic peptide” is any peptide with membrane destabilizing abilities. In general, fusogenic peptides have the propensity to form an amphiphilic alpha-helical structure when in the presence of a hydrophobic surface such as a membrane. The presence of a fusogenic peptide induces formation of pores in the cell membrane by disruption of the ordered packing of the membrane phospholipids. Some fusogenic peptides act to promote lipid disorder and in this way enhance the chance of merging or fusing of proximally positioned membranes of two membrane enveloped particles of various nature (e.g. cells, enveloped viruses, liposomes). Other fusogenic peptides may simultaneously attach to two membranes, causing merging of the membranes and promoting their fusion into one. Examples of fusogenic peptides include a fusion peptide from a viral envelope protein ectodomain, a membrane-destabilizing peptide of a viral envelope protein membrane-proximal domain from the cytoplasmic tails.

Other fusogenic peptides often also contain an amphiphilic-region. Examples of amphiphilic-region containing peptides include: melittin, magainins, the cytoplasmic tail of HIV1 gp41, microbial and reptilian cytotoxic peptides such as bomolitin 1, pardaxin, mastoparan, crabrolin, cecropin, entamoeba, and staphylococcal .alpha.-toxin; viral fusion peptides from (1) regions at the N terminus of the transmembrane (TM) domains of viral envelope proteins, e.g. HIV-1, SIV, influenza, polio, rhinovirus, and coxsackie virus; (2) regions internal to the TM ectodomain, e.g. semliki forest virus, sindbis virus, rota virus, rubella virus and the fusion peptide from sperm protein PH-30: (3) regions membrane-proximal to the cytoplasmic side of viral envelope proteins e.g. in viruses of avian leukosis (ALV), Feline immunodeficiency (FIV), Rous Sarcoma (RSV), Moloney murine leukemia virus (MoMuLV), and spleen necrosis (SNV).

In particular embodiments, a functional molecule such as a CPP is covalently linked to DSPE-PEG-maleimide functionalized particles such as PBAE/PLGA blended particles using known methods such as those described in Fields, et al., J Control Release, 164(1):41-48 (2012). For example, DSPE-PEG-function molecule can be added to the 5.0% PVA solution during formation of the second emulsion. In some embodiments, the loading ratio is about 5 nmol/mg ligand-to-polymer ratio.

In some embodiments, the functional molecule is a CPP such as those above, or mTAT (HIV-1 (with histidine modification) HHHHRKKRRQRRRRHHHHH (SEQ ID NO:10) (Yamano, et al., J Control Release, 152:278-285 (2011)); or bPrPp (Bovine prion)

(SEQ ID NO: 11)
MVKSKIGSWILVLFVAMWS DVGLCKKRPKP

(Magzoub, et al., Biochem Biophys Res Commun., 348:379-385 (2006)); or MPG (Synthetic chimera: SV40 Lg T. Ant.+HIV gb41 coat) GALFLGFLGAAGSTMGAWSQPKKKRKV (SEQ ID NO:12) (Endoh, et al., Adv Drug Deliv Rev., 61:704-709 (2009)).

### IX. Methods of Manufacture

A. Methods of Making Particles

The particle compositions described herein can be prepared by a variety of methods.

1. Polycations

In some embodiments, the nucleic acid is first complexed to a polycation. Complexation can be achieved by mixing the nucleic acids and polycations at an appropriate molar ratio. When a polyamine is used as the polycation species, it is useful to determine the molar ratio of the polyamine nitrogen to the polynucleotide phosphate (N/P ratio). In a preferred embodiment, nucleic acids and polyamines are mixed together to form a complex at an N/P ratio of between approximately 8:1 to 15:1. The volume of polyamine solution required to achieve particular molar ratios can be determined according to the following formula:

$${V}_{NH 2} = \frac{{C}_{nucacid , final} x {M}_{w , nucacid} / {C}_{nucacid , final} x {M}_{w , PXΦN : PXΦ} {V}_{final}}{{C}_{NH2} / {M}_{w , NH2}}$$

where M_{w, nucacid}=molecular weight of nucleic acid, M_{w,P}=molecular weight of phosphate groups of the nucleic acid, Φ_{N:P}=N:P ratio (molar ratio of nitrogens from polyamine to the ratio of phosphates from the nucleic acid), C_{NH2}, stock=concentration of polyamine stock solution, and M_{w,NH2}=molecular weight per nitrogen of polyamine

Polycation complexation with nucleic acids can be achieved by mixing solutions containing polycations with solutions containing nucleic acids. The mixing can occur at any appropriate temperature. In one embodiment, the mixing occurs at room temperature. The mixing can occur with mild agitation, such as can be achieved through the use of a rotary shaker.

2. Exemplary Preferred Methods of Manufacture

In preferred embodiments, the particles are formed by a double-emulsion solvent evaporation technique, such as is disclosed in U.S. Published Application No. 2011/0008451 or U.S. Published Application No. 2011/0268810, each of which is a specifically incorporated by reference in its entirety, or Fahmy, et al., Biomaterials, 26:5727-5736, (2005), or McNeer, et al., Mol. Ther. 19, 172-180 (2011)). In this technique, the nucleic acids or nucleic acid/polycation complexes are reconstituted in an aqueous solution. Nucleic acid and polycation amounts are discussed in more detail below and can be chosen, for example, based on amounts and ratios disclosed in U.S. Published Application No. 2011/0008451 or U.S. Published Application No. 2011/0268810, or used by McNeer, et al., (McNeer, et al., Mol. Ther. 19, 172-180 (2011)), or by Woodrow et al. for small interfering RNA encapsulation (Woodrow, et al., Nat Mater, 8:526-533 (2009)). This aqueous solution is then added dropwise to a polymer solution of a desired polymer dissolved in an organic solvent to form the first emulsion.

This mixture is then added dropwise to solution containing a surfactant, such as polyvinyl alcohol (PVA) and sonicated to form the double emulsion. The final emulsion is then poured into a solution containing the surfactant in an aqueous solution and stirred for a period of time to allow the dichloromethane to evaporate and the particles to harden. The concentration of the surfactant used to form the emulsion, and the sonication time and amplitude can been optimized according to principles known in the art for formulating particles with a desired diameter. The particles can be collected by centrifugation. If it is desirable to store the particles for later use, they can be rapidly frozen, and lyophilized.

In preferred embodiments the particles are PLGA particles. In a particular exemplary protocol, nucleic acid (such as PNA, DNA, or PNA-DNA) with or without a polycation (such as spermidine) are dissolved in DNAse/RNAse free H₂O. Encapsulant in H₂O can be added dropwise to a polymer solution of 50:50 ester-terminated PLGA dissolved in dichloromethane (DCM), then sonicated to form the first emulsion. This emulsion can then be added dropwise to 5% polyvinyl alcohol, then sonicated to form the second emulsion. This mixture can be poured into 0.3% polyvinyl alcohol, and stirred at room temperature to form particles. Particles can then be collected and washed with, for example H₂O, collected by centrifugation, and then resuspended in H₂O, frozen at −80° C., and lyophilized Particles can be stored at −20° C. following lyophilization.

Additional techniques for encapsulating the nucleic acid and polycation complex into polymeric particles are described below.

3. Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting particles are washed with water and dried overnight in a lyophilizer. Particles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which can be performed in completely anhydrous organic solvents, are more useful.

4. Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

5. Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble active agent particles in a polymeric solution for a period of time ranging from 0.5 hours to several months. Stabilizing an insoluble pigment and polymer within the dispersed phase (typically a volatile organic solvent) can be useful for most methods of microencapsulation that are dependent on a dispersed phase, including film casting, solvent evaporation, solvent removal, spray drying, phase inversion, and many others.

The stabilization of insoluble active agent particles within the polymeric solution could be critical during scale-up. By stabilizing suspended active agent particles within the dispersed phase, the particles can remain homogeneously dispersed throughout the polymeric solution as well as the resulting polymer matrix that forms during the process of microencapsulation.

Solvent evaporation microencapsulation (SEM) have several advantages. SEM allows for the determination of the best polymer-solvent-insoluble particle mixture that will aid in the formation of a homogeneous suspension that can be used to encapsulate the particles. SEM stabilizes the insoluble particles or pigments within the polymeric solution, which will help during scale-up because one will be able to let suspensions of insoluble particles or pigments sit for long periods of time, making the process less time-dependent and less labor intensive. SEM allows for the creation of particles that have a more optimized release of the encapsulated material.

6. Hot Melt Microencapsulation

In this method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between 0.5 to 1000 microns are obtained with this method. The external surfaces of spheres prepared with this technique are usually smooth and dense. This procedure is used to prepare particles made of polyesters and polyanhydrides. However, this method is limited to polymers with molecular weights between 1,000-50,000.

7. Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

8. Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

9. Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

10. Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

11. Solvent removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

12. Spray-Drying

In this method, the polymer is dissolved in organic solvent. A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm Particles ranging between 1-10 microns are obtained with a morphology which depends on the type of polymer used.

13. Nanoprecipitation

In nanoprecipitation, the polymer and nucleic acids are co-dissolved in a selected, water-miscible solvent, for example DMSO, acetone, ethanol, acetone, etc. In a preferred embodiment, nucleic acids and polymer are dissolved in DMSO. The solvent containing the polymer and nucleic acids is then drop-wise added to an excess volume of stirring aqueous phase containing a stabilizer (e.g., poloxamer, Pluronic®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer particles are collected and optionally filtered. Nanoprecipitation-based techniques are discussed in, for example, U.S. Pat. No. 5,118,528.

Advantages to nanoprecipitation include: the method can significantly increase the encapsulation efficiency of drugs that are polar yet water-insoluble, compared to single or double emulsion methods (Alshamsan, Saudi Pharmaceutical Journal, 22(3):219-222 (2014)). No emulsification or high shear force step (e.g., sonication or high-speed homogenization) is involved in nanoprecipitation, therefore preserving the conformation of nucleic acids. Nanoprecipitation relies on the differences in the interfacial tension between the solvent and the nonsolvent, rather than shear stress, to produce particles. Hydrophobicity of the drug will retain it in the instantly-precipitating particles; the un-precipitated polymer due to equilibrium is “lost” and not in the precipitated particle form.

B. Molecules to be Encapsulated or Attached to the Surface of the Particles

There are two principle groups of molecules to be encapsulated or attached to the polymer, either directly or via a coupling molecule: targeting molecules, attachment molecules and therapeutic, nutritional, diagnostic or prophylactic agents. These can be coupled using standard techniques. The targeting molecule or therapeutic molecule to be delivered can be coupled directly to the polymer or to a material such as a fatty acid which is incorporated into the polymer.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in two ways. The first is during the preparation of the particles, for example during the emulsion preparation of particles by incorporation of stabilizers with functional chemical groups. Example 1 demonstrates this type of process whereby functional amphiphilic molecules are inserted into the particles during emulsion preparation.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This second class also includes a process whereby amphiphilic molecules such as fatty acids, lipids or functional stabilizers may be passively adsorbed and adhered to the particle surface, thereby introducing functional end groups for tethering to ligands.

In the preferred embodiment, the surface is modified to insert amphiphilic polymers or surfactants that match the polymer phase HLB or hydrophile-lipophile balance, as demonstrated in the following example. HLBs range from 1 to 15. Surfactants with a low HLB are more lipid loving and thus tend to make a water in oil emulsion while those with a high HLB are more hydrophilic and tend to make an oil in water emulsion. Fatty acids and lipids have a low HLB below 10. After conjugation with target group (such as hydrophilic avidin), HLB increases above 10. This conjugate is used in emulsion preparation. Any amphiphilic polymer with an HLB in the range 1-10, more preferably between 1 and 6, most preferably between 1 and up to 5, can be used. This includes all lipids, fatty acids and detergents.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

The molecules to be delivered can also be encapsulated into the polymer using double emulsion solvent evaporation techniques, such as that described by Luo et al., Controlled DNA delivery system, Phar. Res., 16: 1300-1308 (1999).

C. Exemplary Particle Formulations

The particle formulation can be selected based on the considerations including the targeted tissue or cells. For example, in embodiments directed to treatment of treating or correcting beta-thalassemia (e.g. when the target cells are, for example, hematopoietic stem cells), a preferred particle formulation is PLGA or PACE.

Other preferred particle formulations, particularly preferred for treating cystic fibrosis, are described in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Such particles are composed of a blend of Poly(beta-amino) esters (PBAEs) and poly(lactic-co-glycolic acid) (PLGA). Poly(beta-amino) esters (PBAEs) are degradable, cationic polymers synthesized by conjugate (Michael-like) addition of bifunctional amines to diacrylate esters (Lynn, Langer R, editor. J Am Chem Soc. 2000. pp. 10761-10768). PBAEs appear to have properties that make them efficient vectors for gene delivery. These cationic polymers are able to condense negatively charged pDNA, induce cellular uptake, and buffer the low pH environment of endosomes leading to DNA escape (Lynn, Langer R, editor. J Am Chem Soc. 2000. pp. 10761-10768, and Green, Acc Chem Res., 41(6):749-759 (2008)). PBAEs have the ability to form hybrid particles with other polymers, which allows for production of solid, stable and storable particles. For example, blending cationic PBAE with PLGA produced highly loaded pDNA particles. The addition of PBAE to PLGA resulted in an increase in gene transfection in vitro and induced antigen-specific tumor rejection in a murine model (Little, et al. Proc Natl Acad Sci USA., 101:9534-9539 (2004), Little, et al., J Control Release, 107:449-462 (2005)).

Therefore, in some embodiments, the particles utilized to deliver the compositions are composed of a blend of PBAE and a second polymer one of those discussed above. In some embodiments, the particles are composed of a blend of PBAE and PLGA.

PLGA and PBAE/PLGA blended particles loaded with gene editing technology can be formulated using a double-emulsion solvent evaporation technique such as that described in detail above, and in McNeer, et al., Nature Commun., 6:6952. doi: 10.1038/ncomms7952 (2015), and Fields, et al., Adv Healthc Mater., 4(3):361-6 (2015). doi: 10.1002/adhm.201400355 (2015) Epub 2014. Poly(beta amino ester) (PBAE) can synthesized by a Michael addition reaction of 1,4-butanediol diacrylate and 4,4′-trimethylenedipiperidine as described in Akinc, et al., Bioconjug Chem., 14:979-988 (2003). In some embodiments, PBAE blended particles such as PLGA/PBAE blended particles, contain between about 1 and 99, or between about 1 and 50, or between about 5 and 25, or between about 5 and 20, or between about 10 and 20, or about 15 percent PBAE (wt %). In particular embodiments, PBAE blended particles such as PLGA/PBAE blended particles, contain about 50, 45, 40, 35, 30, 25, 20, 15, 10, or 5% PBAE (wt %). Solvent from these particles in PVA as discussed above, and in some cases may continue overnight. PLGA/PBAE/MPG nanoparticles was shown to produce significantly greater nanoparticle association with airway epithelial cells than PLGA nanoparticles (Fields, et al., Advanced Healthcare Materials, 4:361-366 (2015)).

### X. Diseases to Be Treated

Therapy including, but not limited to, genetic correction during in vitro embryonic development could provide treatment or cure of numerous diseases and allow normal fetal development. Thus, the method can treat or prevent a disease or disorder in an embryo. The disease or disorder is typically tied to a genetic defect that can be corrected using the non-enzymatic gene editing technology. The disease or disorder can be a fetal disease or disorder.

Suitable subjects include, but are not limited to embryos of mammals such as a human or other primate, a rodent such as a mouse or rat, or an agricultural or domesticated animal such as a dog, cat, cow, horse, pig, or sheep.

Candidates for embryonic gene therapy include diseases corrected by replacement of an inactive or absent protein. Monogenic diseases that pose the risk of serious fetal, neonatal, and pediatric morbidity or mortality are particularly attractive targets for embryonic gene editing. Exemplary disease targets include, but are not limited to, cystic fibrosis, Tay-Sachs disease, hematopoietic stem cell disorders (e.g., sickle cell, thalassemia), and others disclosed herein.

Attractive targets for embryonic gene therapy also include those discussed in Schneider & Coutelle, Nature Medicine, 5, 256-257 (1999), a Table from which is reproduced below.

TABLE 1
Examplary Targets for Gene Editing
Exemplary candidate diseases for prenatal gene therapy

| | Therapeutic gene | |
|---|---|---|
| Disease | product | Target cells |
| Cystic fibrosis | Cystic fibrosis | Bronchial and |
| | transmembrane | intestinal |
| | conductance regulator | epithelial cells |
| Ornithine transcarbanylas | Ornithine | Hepatocytes |
| deficiency | transcarbaronylase | |
| Glycogen storage | α1, 4-glucosidase | muscle cells, |
| disorders: | | hepatocytes, |
| Pompe disease | | neurons |
| Sphingolipid storage | Glucocerebrosidase | Hematopietic |
| disorders: | ß-N- | stem cells, |
| Gaucher disease | acetylhexosaminidase | muscle cells, |
| Tay-Sachs disease | cerebroxide sulfatase | fibroblasts, |
| Metachromatic | | neurons |
| leucodystrophy | | |
| Mucopolysaccharide | | |
| storage disorders: | α-L-idoronidase | hematopoietic |
| Hurler disease | iduronate-2 sulfatase | stem cells, |
| Hunter disease | ß-glucoronidase | fibroblasts, |
| Sky disease | | neurons |
| Muscular dystrophy | Dystrophin | Muscle cells |
| type Duchenne | | |
| α2-Thalassemia | α-globin chains of | fetal blood cells |
| | hemoglobin | |
| Crigler-Najar disease | billrubine | hepatocytes |
| type 1 | glucuronyltransferase | |
| Tyrosinemia type 1 | fumaryfacetoacetate | hepatocytes |
| | lyase | |
| Junctional epidermolysis | laminin-5 chains | keratinocytes |
| bullosa type Herlitz | | |
| Spinal Muscular atrophy | Survival motor neuron | neurons |
| type Werdnig-Hoffmann | protein or neurotrophin-3 | |

Gene therapy is apparent when studied in the context of human genetic diseases, for example, cystic fibrosis, hemophilia, globinopathies such as sickle cell anemia and beta-thalassemia, xeroderma pigmentosum, and lysosomal storage diseases, though the strategies are also useful for treating non-genetic disease such as HIV, in the context of in vitro-based cell modification. The compositions are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

If the target gene is an oncogene causing unregulated proliferation, such as in a cancer cell, then the oligonucleotide is useful for causing a mutation that inactivates the gene and terminates or reduces the uncontrolled proliferation of the cell. The oligonucleotide is also a useful anti-cancer agent for activating a repressor gene that has lost its ability to repress proliferation. The target gene can also be a gene that encodes an immune regulatory factor, such as PD-1, in order to enhance the host's immune response to a cancer.

The compositions can be used as antiviral agents, for example, when designed to modify a specific a portion of a viral genome necessary for proper proliferation or function of the virus.

In some embodiments, the embryo has a genetic mutation that causes hemophilia, a hemoglobinopaty, cystic fibrosis, xeroderma pigmentosum, or a lysosomal storage disease, and the methods include administering an effective amount of a particle-loaded non-enzymatic gene editing composition to correct to the mutation in the embryo.

A. Variants, Substitutions, and Exemplary PNAs

Preferred diseases and sequences of exemplary targeting sites, triplex-forming molecules, and donor oligonucleotides are discussed in more detail below. Any of the sequences can also be modified as disclosed herein or otherwise known in the art. For example, in some embodiments, any of the triplex-forming molecules herein can have one or more mutations (e.g., substitutions, deletions, or insertions), such that the triplex-forming molecules still bind to the target sequence.

Any of the triplex-forming molecules herein can be manufactured using canonical nucleic acids or other suitable substitutes including those disclosed herein (e.g., PNAs), without or without any of the base, sugar, or backbone modifications discussed herein or in WO 1996/040271, WO/2010/123983, and U.S. Pat. No. 8,658,608.

Any of the triplex-forming molecules herein can be peptide nucleic acids. In some embodiments, one or more of the cytosines of any of triplex-forming molecules herein is substituted with a pseudoisocytosine. In some embodiments, all of the cytosines in the Hoogsteen-binding portion of a triplex-forming molecule are substituted with pseudoisocytosine. In some embodiments, any of the triplex-forming molecules herein, includes one or more of peptide nucleic acid residues substituted with side chain (for example: amino acid side chain or miniPEG side chain) at the alpha, beta and/or gamma position of the backbone. For example, the PNA oligomer can comprise at least one residue comprising a gamma modification/substitution of a backbone carbon atom. In some embodiments all of the peptide nucleic acid residues in the Hoogsteen-binding portion only, the Watson-Crick-binding portion only, or across the entire PNA are substituted with γPNA residues. In particular embodiments, alternating residues are PNA and γPNA in the Hoogsteen-binding portion only, the Watson-Crick-binding portion only, or across the entire PNA are substituted. In some embodiments, the modifications are miniPEG γPNA residues, methyl γPNA residues, or another γ substitution discussed above. In some embodiments, the PNA oligomer includes two or more different modifications of the backbone (e.g. two different types of gamma side chains).

In some embodiments, (1) some or all of the residues in the Watson-Crick binding portion are γPNA residues (e g, miniPEG-containing γPNA residues); (2) some or all of the residues in the Hoogsteen binding portion are γPNA residues (e.g., miniPEG-containing γPNA residues); or (3) some or all of the residues (in the Watson-Crick and/or Hoogsteen binding portions) are γPNA residues (e.g., miniPEG-containing γPNA residues). Therefore, in some embodiments any of the triplex-forming molecules herein is a peptide nucleic acid wherein (1) all of the residues in the Watson-Crick binding portion are γPNA residues (e g, miniPEG-containing γPNA residues) and none of the residues is in Hoogsteen binding portion are γPNA residues (e g, miniPEG-containing γPNA residues); (2) all of the residues in the Hoogsteen binding portion are γPNA residues (e g, miniPEG-containing γPNA residues) and none of the residues is in Watson-Crick binding portion are γPNA residues (e.g., miniPEG-containing γPNA residues); or (3) all of the residues (in the Watson-Crick and Hoogsteen binding portions) are γPNA residues (e.g., miniPEG-containing γPNA residues).

In some embodiments, the triplex-forming molecules are bis-peptide nucleic acids or tail-clamp PNAs with pseudoisocytosine substituted for one or more cytosines, particularly in the Hoogsteen-binding portion, and wherein some or all of the PNA residues are γPNA residues.

Any of the triplex-forming molecules herein can have one or more G-clamp-containing residues. For example, one or more cytosines or variant thereof such as pseudoisocytosine in any of the triplex-forming molecules herein can be substituted or otherwise modified to be a clamp-G (9-(2-guanidinoethoxy) phenoxazine).

Any of the triplex-forming molecules herein can include a flexible linker, linking, for example, a Hoogsteen-binding domain and a Watson-Crick binding domain to form a bis-PNA or tcPNA. The sequences can be linked with a flexible linker. For example, in some embodiments the flexible linker includes about 1-10, more preferably 2-5, most preferably about 3 units such as 8-amino-2, 6, 10-trioxaoctanoic acid residues. Some molecules include N-terminal or C-terminal non-binding residues, preferably positively charged residues. For example, some molecules include 1-10, preferably 2-5, most preferably about 3 lysines at the N-terminus, the C-terminus, or at both the N-terminus and the C-terminus.

For the disclosed sequences, “J” is pseudoisocytosine, “O” can be a flexible 8-amino-3,6-dioxaoctanoic acid, 6-aminohexanoic acid, or 8-amino-2, 6, 10-trioxaoctanoic acid moiety, “K” and “lys” (or “Lys”) are lysine.

PNA sequence are generally presented in N-terminal-to-C-terminal orientation.

In some embodiments, PNA sequences can be presented in the form: H-“nucleobase sequence”-NH₂ orientation, wherein the H represents the N-terminal hydrogen atom of an unmodified PNA oligomer and the —NH₂ represents the C-terminal amide of the polymer. For bis-PNA and tcPNA, the Hoosten-binding portion can be oriented up stream (e.g., at the “H” or N-terminal end of the polyamide) of the linker, while the Watson-Crick-binding portion can be oriented downstream (e.g., at the NH₂ (C-terminal) end) of the polymer/linker.

Any of the donor oligonucleotides can include optional phosphorothiate internucleoside linkages, particular between the two, three or four terminal 5′ and two, three or four terminal 3′ nucleotides. In some embodiments, the phosphorothioate internucleotide linkages need not be sequential and can be dispersed within the donor oligonucleotide. Nevertheless, the phosphorothioate internucleotide linkages can be oriented primarily near each termini of the donor oligonucleotide. Thus, each of the donor oligonucleotide sequences disclosed herein is expressly disclosed without any phosphorothiate internucleoside linkages, and with phosphorothiate internucleoside linkages, preferably between the two, three or four terminal 5′ and two, three or four terminal 3′ nucleotides.

1. Globinopathies

Worldwide, globinopathies account for significant morbidity and mortality. Over 1,200 different known genetic mutations affect the DNA sequence of the human alpha-like (HBZ, HBA2, HBA1, and HBQ1) and beta-like (HBE1, HBG1, HBD, and HBB) globin genes. Two of the more prevalent and well-studied globinopathies are sickle cell anemia and β-thalassemia. Substitution of valine for glutamic acid at position 6 of the β-globin chain in patients with sickle cell anemia predisposes to hemoglobin polymerization, leading to sickle cell rigidity and vasoocclusion with resulting tissue and organ damage. In patients with β-thalassemia, a variety of mutational mechanisms results in reduced synthesis of β-globin leading to accumulation of aggregates of unpaired, insoluble α-chains that cause ineffective erythropoiesis, accelerated red cell destruction, and severe anemia.

Together, globinopathies represent the most common single-gene disorders in man. Triplex-forming molecules are particularly well suited to treat globinopathies, as they are single gene disorders caused by point mutations. Triplex-forming molecules are effective at binding to the human β-globin both in vitro and in living cells, both ex vivo and in vivo in animals. Experimental results also demonstrate correction of a thalassemia-associated mutation in vivo in a transgenic mouse carrying a human beta globin gene with the IVS2-654 thalassemia mutation (in place of the endogenous mouse beta globin) with correction of the mutation in 4% of the total bone marrow cells, cure of the anemia with blood hemoglobin levels showing a sustained elevation into the normal range, reversal of extramedullary hematopoiesis and reversal of splenomegaly, and reduction in reticulocyte counts, following systemic administration of PNA and DNA containing nanoparticles.

β-thalassemia is an unstable hemoglobinopathy leading to the precipitation of α-hemoglobin within RBCs resulting in a severe hemolytic anemia. Patients experience jaundice and splenomegaly, with substantially decreased blood hemoglobin concentrations necessitating repeated transfusions, typically resulting in severe iron overload with time. Cardiac failure due to myocardial siderosis is a major cause of death from (3-thalassemia by the end of the third decade. Reduction of repeated blood transfusions in these patients is therefore of primary importance to improve patient outcomes.

a. Exemplary β-Globin Gene Target Sites

In the β-globin gene sequence, particularly in the introns, there are many good third-strand binding sites that may be utilized in the methods disclosed herein. A portion of the GenBank sequence of the chromosome-11 human-native hemoglobin-gene cluster (GenBank: U01317.1—Human beta globin region on chromosome 11—LOCUS HUMHBB, 73308 bp ds-DNA) from base 60001 to base 66060 is presented below. The start of the gene coding sequence at position 62187-62189 (or positions 2187-2189 of SEQ ID NO:13) is indicated by wave underlining. This portion of the GenBank sequence contains the native 13 globin gene sequence. In sickle cell hemoglobin the adenine base at position 62206 (or position 2206 as listed in SEQ ID NO:13, indicated in bold and heavy underlining) is mutated to a thymine. Other common point mutations occur in intron 2 (IVS2), which is highlighted in the sequence below by italics (SEQ ID NO:14) and corresponds with nucleotides 2,632-3,481 of SEQ ID NO:13. Mutations include IVS2-1, IVS2-566, IVS2-654, IVS2-705, and IVS2-745, which are also shown in bold and heavy underlining; numbering relative to the start of intron 2.

Exemplary triplex-forming molecule binding sites, are provided in, for example, WO 1996/040271, WO/2010/123983, and U.S. Pat. No. 8,658,608, and in the working Examples below. Target regions can be reference based on the coding strand of genomic DNA, or the complementary non-coding sequence thereto (e.g., the Watson or Crick stand). Exemplary target regions are identified with reference to the coding sequence of the 13 globin gene sequence in the sequence below by double underlining and a combination of underlining and double underlining (wherein the underlining is optional additional binding sequence). Additionally, for each targeting sequence identified, the complementary target sequence on the reverse non-coding strand is also explicitly disclosed as a triplex-forming molecule binding sequence.

Accordingly, triplex-forming molecules can be designed to bind a target region on either the coding or non-coding strand. However, as discussed above, triplex-forming molecules, such as PNA and tcPNA preferably invade the target duplex, displace the polypyrimidine strand, and induce triplex formation with the polypurine strand.

(SEQ ID NO: 13 - full sequence;
SEQ ID NO: 14 - sequence in italics).
AAAGCTCTTGCTTTGACAATTTTGGTCTTTCAGAATACTATAAATATAACC
TATATTATAATTTCATAAAGTCTGTGCATTTTCTTTGACCCAGGATATTTG
CAAAAGACATATTCAAACTTCCGCAGAACACTTTATTTCACATATACATGC
CTCTTATATCAGGGATGTGAAACAGGGTCTTGAAAACTGTCTAAATCTAAA
ACAATGCTAATGCAGGTTTAAATTTAATAAAATAAAATCCAAAATCTAACA
GCCAAGTCAAATCTGTATGTTTTAACATTTAAAATATTTTAAAGACGTCTT
TTCCCAGGATTCAACATGTGAAATCTTTTCTCAGGGATACACGTGTGCCTA
GATCCTCATTGCTTTAGTTTTTTACAGAGGAATGAATATAAAAAGAAAATA
CTTAAATTTTATCCCTCTTACCTCTATAATCATACATAGGCATAATTTTTT
AACCTAGGCTCCAGATAGCCATAGAAGAACCAAACACTTTCTGCGTGTGTG
AGAATAATCAGAGTGAGATTTTTTCACAAGTACCTGATGAGGGTTGAGACA
GGTAGAAAAAGTGAGAGATCTCTATTTATTTAGCAATAATAGAGAAAGCAT
TTAAGAGAATAAAGCAATGGAAATAAGAAATTTGTAAATTTCCTTCTGATA
ACTAGAAATAGAGGATCCAGTTTCTTTTGGTTAACCTAAATTTTATTTCAT
TTTATTGTTTTATTTTATTTTATTTTATTTTATTTTGTGTAATCGTAGTTT
CAGAGTGTTAGAGCTGAAAGGAAGAAGTAGGAGAAACATGCAAAGTAAAAG
TATAACACTTTCCTTACTAAACCGACTGGGTTTCCAGGTAGGGGCAGGATT
CAGAATGACTGACAGGGCCCTTAGGGAACACTGAGACCCTACGCTGACCTC
ATAAATGCTTGCTACCTTTGCTGTTTTAATTACATCTTTTAATAGCAGGAA
GCAGAACTCTGCACTTCAAAAGTTTTTCCTCACCTGAGGAGTTAATTTAGT
ACAAGGGGAAAAAGTACAGGGGGATGGGAGAAAGGCGATCACGTTGGGAAG
CTATAGAGAAAGAAGAGTAAATTTTAGTAAAGGAGGTTTAAACAAACAAAA
TATAAAGAGAAATAGGAACTTGAATCAAGGAAATGATTTTAAAACGCAGTA
TTCTTAGTGGACTAGAGGAAAAAAATAATCTGAGCCAAGTAGAAGACCTTT
TCCCCTCCTACCCCTACTTTCTAAGTCACAGAGGCTTTTTGTTCCCCCAGA
CACTCTTGCAGATTAGTCCAGGCAGAAACAGTTAGATGTCCCCAGTTAACC
TCCTATTTGACACCACTGATTACCCCATTGATAGTCACACTTTGGGTTGTA
AGTGACTTTTTATTTATTTGTATTTTTGACTGCATTAAGAGGTCTCTAGTT
TTTTATCTCTTGTTTCCCAAAACCTAATAAGTAACTAATGCACAGAGCACA
TTGATTTGTATTTATTCTATTTTTAGACATAATTTATTAGCATGCATGAGC
AAATTAAGAAAAACAACAACAAATGAATGCATATATATGTATATGTATGTG
TGTATATATACACATATATATATATATTTTTTTTCTTTTCTTACCAGAAGG
TTTTAATCCAAATAAGGAGAAGATATGCTTAGAACTGAGGTAGAGTTTTCA
TCCATTCTGTCCTGTAAGTATTTTGCATATTCTGGAGACGCAGGAAGAGAT
CCATCTACATATCCCAAAGCTGAATTATGGTAGACAAAGCTCTTCCACTTT
TAGTGCATCAATTTCTTATTTGTGTAATAAGAAAATTGGGAAAACGATCTT
CAATATGCTTACCAAGCTGTGATTCCAAATATTACGTAAATACACTTGCAA
AGGAGGATGTTTTTAGTAGCAATTTGTACTGATGGTATGGGGCCAAGAGAT
ATATCTTAGAGGGAGGCCTGAGCCTTTGAAGTCCAACTCCTAAGCCAGTGC
CAGAAGAGCCAAGGACAGGTACGGCTGTCATCACTTAGACCTCACCCTGTG
GAGCCACACCCTAGGGTTGGCCAATCTACTCCCAGGAGCAGGGAGGGCAGG
AGCCAGGGCTGGGCATAAAAGTCAGGGCAGAGCCATCTATTGCTTACATTT
GCTTCTGACACAACTGTGTTCACTAGCAACCTCAAACAGACACC [symbol] GTGC
ACCTGACTCCTG [symbol] GGAGAAGTCTGCCGTTACTGCCCTGTGGGGCAAGGTGA
ACGTGGATGAAGTTGGTGGTGAGGCCCTGGGCAGGTTGGTATCAAGGTTAC
AAGACAGGTTTAAGGAGACCAATAGAAACTGGGCATGTGGAGACAGAGAAG
ACTCTTGGGTTTCTGATAGGCACTGACTCTCTCTGCCTATTGGTCTATTTT
CCCACCCTTAGGCTGCTGGTGGTCTACCCTTGGACCCAGAGGTTCTTTGAG
TCCTTTGGGGATCTGTCCACTCCTGATGCTGTTATGGGCAACCCTAAGGTG
AAGGCTCATGGCAAGAAAGTGCTCGGTGCCTTTAGTGATGGCCTGGCTCAC
CTGGACAACCTCAAGGGCACCTTTGCCACACTGAGTGAGCTGCACTGTGAC
AAGCTGCACGTGGATCCTGAGAACTTCAGG [symbol] TGAGTCTATGGGACCCTTGA
TG [symbol] ATGGTTAAGTTCATGTCATAGG [symbol] [symbol]
TAACAGGGTACATTTTAGAATGGGAAACAGACGAATGATTGCATCA
GTGTGGAAGTCTCAGGATCGTTTTAGTTTCTTTTATTTGCTGTTCATAACA
ATTGTTTTCTTTTGTTTAA [symbol] GCAAT
TTTTACTATTATACTTAATGCCTTAACATTGTGTATAACAAAAGGAAATAT
CTCTGAGATACATTAAGTAACTTAAAAAAAAACTTTACACAGTCTGCCTAG
TACATTACTATTTGGAATATATGTGTGCTTATTTGCATATTCATAATCTCC
CTACTTTATTTTCTTTTATTTTTAATTGATACATAATCATTATACATATTT
ATGGGTTAAAGTGTAATGTTTTAATATGTGTACACATATTGACCAAATCAG
GGTAATTTTGCATTTGTAATTTTAAAAAATG [symbol] AATATACT
TTTTTGTTTATCTTATTTCTAATA [symbol]
AATAATGATACAATGTATCATG [symbol] AAAGAATAACAG
TGATAATTTCTGGGTTAAGG [symbol] AATAGCAATATTTCTGCATATAAATATTTC
TGCATATAAATTGTAACTGA [symbol] GTAAGAGGTTTCATATTGCTAATAGCAGCT
ACAATCCAG [symbol] TACCATTCTGCTTTTATTTTATGGTTGGGATAAGGCTGGAT
TATTCTGAGTCCAACTAGG [symbol] TCATACCTCTTTA [symbol] [symbol]
ACAGCTCCTGGGCAACGTGCTGGTCTGTGTGCTGGCCCATCA
CTTTGGCAAAGAATTCACCCCACCAGTGCAGGCTGCCTATCAGAAAGTGGT
GGCTGGTGTGGCTAATGCCCTGGCCCACAAGTATCACTAAGCTCGCTTTCT
TGCTGTCCAATTTCTATTAAAGGTTCCTTTGTTCCCTAAGTCCAACTACTA
AACTGGGGGATATTATCAAGGGCCTTGAGCATCTGGATTCTGCCTAATAAA
AAACATTTATTTTCATTGCAATGATGTATTTAAATTATTTCTGAATATTTT
ACTAAAAAGGGAATGTGGGAGGTCAGTGCATTTAAAACATAAAGAAATGAA
GAGCTAGTTCAAACCTTGGGAAAATACACTATATCTTAAACTCCATGAAAG
AAGGTGAGGCTGCAAACAGCTAATGCACATTGGCAACAGCCCTGATGCCTA
TGCCTTATTCATCCCTCAGAAAAGGATTCAAGTAGAGGCTTGATTTGGAGG
TTAAAGTTTTGCTATGCTGTATTTTACATTACTTATTGTTTTAGCTGTCCT
CATGAATGTCTTTTCACTACCCATTTGCTTATCCTGCATCTCTCAGCCTTG
ACTCCACTCAGTTCTCTTGCTTAGAGATACCACCTTTCCCCTGAAGTGTTC
CTTCCATGTTTTACGGCGAGATGGTTTCTCCTCGCCTGGCCACTCAGCCTT
AGTTGTCTCTGTTGTCTTATAGAGGTCTACTTGAAGAAGGAAAAACAGGGG
GCATGGTTTGACTGTCCTGTGAGCCCTTCTTCCCTGCCTCCCCCACTCACA
GTGACCCGGAATCTGCAGTGCTAGTCTCCCGGAACTATCACTCTTTCACAG
TCTGCTTTGGAAGGACTGGGCTTAGTATGAAAAGTTAGGACTGAGAAGAAT
TTGAAAGGGGGCTTTTTGTAGCTTGATATTCACTACTGTCTTATTACCCTA
TCATAGGCCCACCCCAAATGGAAGTCCCATTCTTCCTCAGCATGTTTAAGA
TTAGCATTCAGGAAGAGATCAGAGGTCTGCTGGCTCCCTTATCATGTCCCT
TATGGTGCTTCTGGCTCTGCAGTTATTAGCATAGTGTTACCATCAACCACC
TTAACTTCATTTTTCTTATTCAATACCTAGGTAGGTAGATGCTAGATTCTG
GAAATAAAATATGAGTCTCAAGTGGTCCTTGTCCTCTCTCCCAGTCAAATT
CTGAATCTAGTTGGCAAGATTCTGAAATCAAGGCATATAATCAGTAATAAG
TGATGATAGAAGGGTATATAGAAGAATTTTATTATATGAGAGGGTGAAACC
TAAAATGAAATGAAATCAGACCCTTGTCTTACACCATAAACAAAAATAAAT
TTGAATGGGTTAAAGAATTAAACTAAGACCTAAAACCATAAAAATTTTTAA
AGAAATCAAAAGAAGAAAATTCTAATATTCATGTTGCAGCCGTTTTTTGAA
TTTGATATGAGAAGCAAAGGCAACAAAAGGAAAAATAAAGAAGTGAGGCTA
CATCAAACTAAAAAATTTCCACACAAAAAAGAAAACAATGAACAAATGAAA
GGTGAACCATGAAATGGCATATTTGCAAACCAAATATTTCTTAAATATTTT
GGTTAATATCCAAAATATATAAGAAACACAGATGATTCAATAACAAACAAA
AAATTAAAAATAGGAAAATAAAAAAATTAAAAAGAAGAAAATCCTGCCATT
TATGCGAGAATTGATGAACCTGGAGGATGTAAAACTAAGAAAAATAAGCCT
GACACAAAAAGACAAATACTACACAACCTTGCTCATATGTGAAACATAAAA
AAGTCACTCTCATGGAAACAGACAGTAGAGGTATCCTTTCCAGGGGTTGGG
GGTGGGAGAATCAGGAAACTATTACTCAAAGGGTATAAAATTTCAGTTATG
TGGGATGAATAAATTCTAGATATCTAATGTACAGCATCGTGACTGTAGTTA
ATTGTACTGTAAGTATATTTAAAATTTGCAAAGAGAGTAGATTTTTTTGTT
TTTTTAGATGGAGTTTTGCTCTTGTTGTCCAGGCTGGAGTGCAATGGCAAG
ATCTTGGCTCACTGCAACCTCCGCCTCCTGGGTTCAAGCAAATCTCCTGCC
TCAGCCTCCCGAGTAGCTGGGATTACAGGCATGCGACACCATGCCCAGCTA
ATTTTGTATTTTTAGTAGAGACGGGGTTTCTCCATGTTGGTCAGGCTGATC
CGCCTCCTCGGCCACCAAAGGGCTGGGATTACAGGCGTGACCACCGGGCCT
GGCCGAGAGTAGATCTTAAAAGCATTTACCACAAGAAAAAGGTAACTATGT
GAGATAATGGGTATGTTAATTAGCTTGATTGTGGTAATCATTTCACAAGGT
ATACATATATTAAAACATCATGTTGTACACCTTAAATATATACAATTTTTA
TTTGTGAATGATACCTCAATAAAGTTGAAGAATAATAAAAAAGAATAGACA
TCACATGAATTAAAAAACTAAAAAATAAAAAAATGCATCTTGATGATTAGA
ATTGCATTCTTGATTTTTCAGATACAAATATCCATTTGACTG

b. Exemplary Triplex-Forming Sequences

i. Beta Thalassemia

Gene editing molecules can be designed based on the guidance provided herein and otherwise known in the art. Exemplary triplex-forming molecule and donor sequences, are provided in, for example, WO 1996/040271, WO/2010/123983, and U.S. Pat. No. 8,658,608, and in the working Examples below, and can be altered to include one or more of the modifications disclosed herein.

Triplex-forming molecules can include a sequence substantially complementary to the polypurine strand of the polypyrimidine:polypurine target motif. In some embodiments, the triplex-forming molecules target a region corresponding to nucleotides 566-577, optionally 566-583 or more of SEQ ID NO:14; a region corresponding to nucleotides 807-813, optionally 807-824 or more of SEQ ID NO:14; or a region corresponding to nucleotides 605-611, optionally 605-621 of SEQ ID NO:14. Therefore in some embodiments, the triplex-forming molecules can form a triple-stranded molecule with the sequence including GAAAGAAAGAGA (SEQ ID NO:15) or TGCCCTGAAAGAAAGAGA (SEQ ID NO:16) or GGAGAAA or AGAATGGTGCAAAGAGG (SEQ ID NO:17) or AAAAGGG or ACATGATTAGCAAAAGGG (SEQ ID NO:18).

Accordingly, in some embodiments, the triplex-forming molecule includes the nucleic acid sequence CTTTCTTTCTCT (SEQ ID NO:19), preferably includes the sequence CTTTCTTTCTCT (SEQ ID NO:19) linked to the sequence TCTCTTTCTTTC (SEQ ID NO:20), or more preferably includes the sequence CTTTCTTTCTCT (SEQ ID NO:19) linked to the sequence TCTCTTTCTTTCAGGGCA (SEQ ID NO:21).

In some embodiments, the triplex-forming molecule includes the nucleic acid sequence TTTCCC, preferably includes the sequence TTTCCC linked to the sequence CCCTTTT, or more preferably includes the sequence TTTCCC linked to the sequence CCCTTTTGCTAATCATGT (SEQ ID NO:22).

In some embodiments, the triplex-forming molecule includes the nucleic acid sequence TTTCTCC, preferably includes the sequence TTTCTCC linked to the sequence CCTCTTT, or more preferably includes the sequence TTTCTCC linked to the sequence CCTCTTTGCACCATTCT (SEQ ID NO:23).

In some preferred embodiments, the triplex-forming nucleic acid is a peptide nucleic acid including the sequence JTTTJTTTJTJT (SEQ ID NO:24) linked to the sequence TCTCTTTCTTTC (SEQ ID NO:20) or TCTCTTTCTTTCAGGGCA (SEQ ID NO:21); or

a peptide nucleic acid including the sequence TTTTJJJ linked to the sequence CCCTTTT or CCCTTTTGCTAATCATGT (SEQ ID NO:22);

or a peptide nucleic acid including the sequence TTTJTJJ linked to the sequence CCTCTTT or CCTCTTTGCACCATTCT (SEQ ID NO:23),

optionally, but preferably wherein one or more of the PNA residues is a γPNA.

In specific embodiments, the triplex-forming molecule is a peptide nucleic acid including the sequence lys-lys-lys-JTTTJTTTJTJT-OOO-TCTCTTTCTTTCAGGGCA-lys-lys-lys (SEQ ID NO:66), or

lys-lys-lys-TTTTJJJ-OOO-CCCTTTTGCTAATCATGT-lys-lys-lys (SEQ ID NO:67), or

lys-lys-lys-TTTJTJJ-OOO-CCTCTTTGCACCATTCT-lys-lys-lys (SEQ ID NO:68);

optionally, but preferably wherein one or more of the PNA residues is a γPNA. In even more specific embodiments, the bolded and underlined residues are miniPEG-containing γPNA.

In other embodiments, the triplex-forming nucleic acid is a peptide nucleic acid including the sequence TJTTTTJTTJ (SEQ ID NO:25) linked to the sequence CTTCTTTTCT (SEQ ID NO:26); or

TTJTTJTTTJ (SEQ ID NO:27) linked to the sequence CTTTCTTCTT (SEQ ID NO:28); or

JJJTJJTTJT (SEQ ID NO:29) linked to the sequence TCTTCCTCCC (SEQ ID NO:30); or

optionally, but preferably wherein one or more of the PNA residues is a γPNA.

In specific embodiments, the triplex-forming nucleic acid is a peptide nucleic acid including the sequence lys-lys-lys-TJTTTTJTTJ-OOO-CTTCTTTTCT-lys-lys-lys (SEQ ID NO:69) (IVS2-24); or

lys-lys-lys-TTJTTJTTTJ-OOO-CTTTCTTCTT-lys-lys-lys (SEQ ID NO:70) (IVS2-512); or

lys-lys-lys-JJJTJJTTJT-OOO-TCTTCCTCCC-lys-lys-lys (SEQ ID NO:71) (IVS2-830);

optionally, but preferably wherein one or more of the PNA residues is a γPNA. In even more specific embodiments, the bolded and underlined residues are miniPEG-containing γPNA.

ii. Sickle Cell Disease

Preferred sequences that target the sickle cell disease mutation (20) in the beta globin gene are also provided. In some embodiments, the triplex-forming molecule includes the nucleic acid sequence CCTCTTC, preferably includes the sequence CCTCTTC linked to the sequence CTTCTCC, or more preferably includes the sequence CCTCTTC linked to the sequence CTTCTCCACAGGAGT (SEQ ID NO:31) or CTTCTCCACAGGAGTCAG (SEQ ID NO:32) or CTTCTCCACAGGAGTCAGGTGC (SEQ ID NO:33).

In some embodiments, the triplex-forming molecule includes the nucleic acid sequence TTCCTCT, preferably includes the sequence TTCCTCT linked to the sequence TCTCCTT, or more preferably includes the sequence TTCCTCT linked to the sequence TCTCCTTAAACCTGT (SEQ ID NO:34) or TCTCCTTAAACCTGTCTT (SEQ ID NO:35).

In some embodiments, the triplex-forming molecule includes the nucleic acid sequence TCTCTTCT, preferably includes the sequence TCTCTTCT linked to the sequence TCTTCTCT, or more preferably includes the sequence TCTCTTCT linked to the sequence TCTTCTCTGTCTCCAC (SEQ ID NO:36) or TCTTCTCTGTCTCCACAT (SEQ ID NO:37).

In some preferred embodiments for correction of Sickle Cell Disease Mutation, the triplex-forming nucleic acid is a peptide nucleic acid including the sequence JJTJTTJ linked to the sequence CTTCTCC or CTTCTCCACAGGAGT (SEQ ID NO:31) or CTTCTCCACAGGAGTCAG (SEQ ID NO:32) or CTTCTCCACAGGAGTCAGGTGC (SEQ ID NO:33);

or a peptide nucleic acid including the sequence TTJJTJT linked to the sequence TCTCCTT or TCTCCTTAAACCTGT (SEQ ID NO:34) or TCTCCTTAAACCTGTCTT (SEQ ID NO:35);

or a peptide nucleic acid including the sequence TJTJTTJT linked to the sequence TCTTCTCT or TCTTCTCTGTCTCCAC (SEQ ID NO:36) or TCTTCTCTGTCTCCACAT (SEQ ID NO:37);

optionally, but preferably wherein one or more of the PNA residues is a γPNA.

In specific embodiments for correction of Sickle Cell Disease Mutation, the triplex-forming nucleic acid is a peptide nucleic acid including the sequence

(SEQ ID NO: 72)
lys-lys-lys-JJTJTTJ-OOO-CTTCTCCACAGGAGT-lys-lys-
lys;
or
(SEQ ID NO: 73)
lys-lys-lys-TTJJTJT-OOO-TCTCCTTAAACCTGT-lys-lys-
lys;
or
(SEQ ID NO: 74)
lys-lys-lys-TTJJTJT-OOO-TCTCCTTAAACCTGTCTT-lys-
lys-lys
(SEQ ID NO: 75)
lys-lys-lys-TJTJTTJT-OOO-TCTTCTCTGTCTCCAC-lys-
lys-lys (tc816);
or
(SEQ ID NO: 76)
lys-lys-lys-JJTJTTJ-OOO-CTTCTCCACAGGAGTCAG-lys-
lys-lys;
or
(SEQ ID NO: 76)
lys-lys-lys-JJTJTTJ-OOO-CTTCTCCACAGGAGTCAG-lys-
lys-lys (SCD-tcPNA 1A);
or
(SEQ ID NO: 76)
lys-lys-lys-JJTJTTJ-OOO-CTTCTCCACAGGAGTCAG-lys-
lys-lys (SCD-tcPNA 1B);
or
(SEQ ID NO: 76)
lys-lys-lys-JJTJTTJ-OOO-CTTCTCCACAGGAGTCAG-lys-
lys-lys (SCD-tcPNA 1C);
or
(SEQ ID NO: 77)
1ys-1ys-1ys-JJTJTTJ-OOO-CTTCTCCACAGGAGTCAGGTGC-
1ys-lys-lys (SCD-tcPNA 1D);
or
(SEQ ID NO: 77)
lys-lys-lys-JJTJTTJ-OOO-CTTCTCCACAGGAGTCAGGTGC-
lys-lys-lys (SCD-tcPNA 1E);
or
(SEQ ID NO: 77)
lys-lys-lys-JJTJTTJ-OOO-CTTCTCCACAGGAGTCAGGTGC-
lys-lys-lys (SCD-tcPNA 1F);
or
(SEQ ID NO: 78)
lys-lys-lys-TJTJTTJT-OOO-TCTTCTCTGTCTCCACAT-lys-
lys-lys;

optionally, but preferably wherein one or more of the PNA residues is a γPNA. In even more specific embodiments, the bolded and underlined residues are miniPEG-containing γPNA.

c. Exemplary Donors

In some embodiments, the triplex-forming molecules are used in combination with a donor oligonucleotide for correction of IVS2-654 mutation that includes the sequence 5′AAAGAATAACAGTGATAATTTCTGGGTTAAGGCAATAGCAATA TCTCTGCATATAAATAT 3′ (SEQ ID NO:93) with the correcting IVS2-654 nucleotide underlined, or a functional fragment thereof that is suitable and sufficient to correct the IVS2-654 mutation.

Other exemplary donor sequences include, but are not limited to, DonorGFP-IVS2-1 (Sense) 5′-GTTCAGCGTGTCCGGCGAGGGCG AGGTGAGTCTATGGGACCCTTGATGTTT-3′ (SEQ ID NO:111), DonorGFP-IVS2-1 (Antisense) 5′-AAACATCAAGGGTCCCATA GACTCACCTCGCCCTCGCCGGACACGCTGAAC-3′ (SEQ ID NO:112), and, or a functional fragment thereof that is suitable and sufficient to correct a mutation.

In some embodiments, a Sickle Cells Disease mutation can be corrected using a donor having the sequence

5′CTTGCCCCACAGGGCAGTAACGGCAGATTTTTC [symbol] CGG CGTTAAATGCACCATGGTGTCTGTTTGAGGT 3′ (SEQ ID NO:94), or a functional fragment thereof that is suitable and sufficient to correct a mutation, wherein the three boxed nucleotides represent the corrected codon 6 which reverts the mutant Valine (associated with human sickle cell disease) back to the wildtype Glutamic acid and nucleotides in bold font (without underlining) represent changes to the genomic DNA but not to the encoded amino acid; or

5′ACAGACACCATGGTGCACCTGACTCCTGAGGAGAAGTCT GCCGTTACTGCC 3′ (SEQ ID NO:95), or a functional fragment thereof that is suitable and sufficient to correct a mutation, wherein the bolded and underlined residue is the correction, or

5′T(s)T(s)G(s)CCCCACAGGGCAGTAACGGCAGACTTCTCCTC AGGAGTCAGGTGCACCATGGTGTCTGT(s)T(s)T(s)G3′ (SEQ ID NO:96), or a functional fragment thereof that is suitable and sufficient to correct a mutation, wherein the bolded and underlined residue is the correction and “(s)” indicates an optional phosphorothiate internucleoside linkage.

2. Cystic Fibrosis

The disclosed compositions and methods can be used to treat cystic fibrosis. Cystic fibrosis (CF) is a lethal autosomal recessive disease caused by defects in the cystic fibrosis transmembrane conductance regulator (CFTR), an ion channel that mediates Cl— transport. Lack of CFTR function results in chronic obstructive lung disease and premature death due to respiratory failure, intestinal obstruction syndromes, exocrine and endocrine pancreatic dysfunction, and infertility (Davis, et al., Pediatr Rev., 22(8):257-64 (2001)). The most common mutation in CF is a three base-pair deletion (F508del) resulting in the loss of a phenylalanine residue, causing intracellular degradation of the CFTR protein and lack of cell surface expression (Davis, et al., Am J Respir Crit Care Med., 173(5):475-82 (2006)). In addition to this common mutation there are many other mutations that occur and lead to disease including a class of mutations due to premature stop codons, nonsense mutations. In fact nonsense mutations account for approximately 10% of disease causing mutations. Of the nonsense mutations G542X and W1282X are the most common with frequencies of 2.6% and 1.6% respectfully.

Although CF is one of the most rigorously characterized genetic diseases, current treatment of patients with CF focuses on symptomatic management rather than primary correction of the genetic defect. Gene therapy has remained an elusive target in CF, because of challenges of in vivo delivery to the lung and other organ systems (Armstrong, et al., Archives of disease in childhood (2014) doi: 10.1136/archdischild-2012-302158. PubMed PMID: 24464978). In recent years, there have been many advances in gene therapy for treatment of diseases involving the hematolymphoid system, where harvest and ex vivo manipulation of cells for autologous transplantation is possible: some examples include the use of zinc finger nucleases targeting CCR5 to produce HIV-1 resistant cells (Holt, et al., Nature biotechnology, 28(8):839-47 (2010)) correction of the ABCD1 gene by lentiviral vectors for treatment of adrenoleukodystrophy (Cartier, et al., Science, 326(5954):818-23 (2009)) and correction of SCID due to ADA deficiency using retroviral gene transfer (Aiuti, et al., The New England Journal Of Medicine, 360(5):447-58 (2009).

Harvest and autologous transplant is not an option in CF, due to the involvement of the lung and other internal organs. As one approach, the UK Cystic Fibrosis Gene Therapy Consortium has tested liposomes to deliver plasmids containing cDNA encoding CFTR to the lung (Alton, et al., Thorax, 68(11):1075-7 (2013)), Alton, et al., The Lancet Respiratory Medicine, (2015). doi: 10.1016/S2213-2600(15)00245-3. PubMed PMID: 26149841.) other clinical trials have used viral vectors for delivery of the CFTR gene or CFTR expression plasmids that are compacted by polyethylene glycol-substituted lysine 30-mer peptides with limited success (Konstan, et al., Human Gene Therapy, 15(12):1255-69 (2004)). Moreover, delivery of plasmid DNA for gene addition without targeted insertion does not result in correction of the endogenous gene and is not subject to normal CFTR gene regulation, and virus-mediated integration of the CFTR cDNA could introduce the risk of non-specific integration into important genomic sites.

In CF, for example, there is evidence of significant multisystem organ damage at birth. The compositions can be administered in an effective amount to induce or enhance gene correction in an amount effective to reduce one or more symptoms of cystic fibrosis.

Sequences for the human cystic fibrosis transmembrane conductance regulator (CFTR) are known in the art, see, for example, GenBank Accession number: AH006034.1, and compositions and methods of targeted correction of CFTR are described in McNeer, et al., Nature Communications, 6:6952, (DOI 10.1038/ncomms7952), 11 pages.

a. Exemplary F508del Target Sites

In some embodiments, the triplex-forming molecules are designed to target the CFTR gene at nucleotides 9,152-9,159 (TTTCCTCT) or 9,159-9,168 (TTTCCTCTATGGGTAAG (SEQ ID NO:38)) of accession number AH006034.1, or the non-coding strand (e.g., 3′-5′ complementary sequence) corresponding to nucleotides 9,152-9,159 or 9,152-9,168 (e.g., 5′-AGAGGAAA-3′, or 5′-CTTACCCATAGAGGAAA-3′ (SEQ ID NO:39)).

In some embodiments, the triplex-forming molecules are designed to target the CFTR gene at nucleotides 9,039-9,046 (5′-AGAAGAGG-3′, or 9,030-9,046 (5′-ATGCCAACTAGAAGAGG-3′ (SEQ ID NO:40)) of accession number AH006034.1, or the non-coding strand (e.g., 3′-5′ complementary sequence) corresponding to nucleotides (5′ CCTCTTCT 3′) or (5′ CCTCTTCTAGTTGGCAT 3′ (SEQ ID NO:41).

In some embodiments, the triplex-forming molecules are designed to target the CFTR gene at nucleotides 8,665-8,683 (CTTTCCCTT) or 8,665-8,682 (CTTTCCCTTGTATCTTTT (SEQ ID NO:42) of accession number AH006034.1, or the non-coding strand (e.g., 3′-5′ complementary sequence) corresponding to nucleotides 8,665-8,683 or 8,665-8,682 (e.g., 5′-AAGGGAAAG-3′, or 5′-AAAAGATACAAGGGAAAG-3′ (SEQ ID NO:43)).

In some embodiments, the triplex-forming molecules are designed to target the W1282X mutation in CFTR gene at the sequence GAAGGAGAAA (SEQ ID NO:44), AAAAGGAA, or AGAAAAAAGG (SEQ ID NO:45), or the inverse complement thereof.

In some embodiments, the triplex-forming molecules are designed to target the G542X mutation in CFTR gene at the sequence AGAAAAA, AGAGAAAGA, or AAAGAAA, or the inverse complement thereof.

b. Exemplary Triplex-Forming Sequences and Donors

i. F508del

In some embodiments, the triplex-forming molecule includes the nucleic acid sequence includes TCTCCTTT, preferably linked to the sequence TTTCCTCT or more preferably includes TCTCCTTT linked to the sequence TTTCCTCTATGGGTAAG (SEQ ID NO:46); or

includes TCTTCTCC preferably linked to the sequence CCTCTTCT, or more preferably includes TCTTCTCC linked to CCTCTTCTAGTTGGCAT (SEQ ID NO:41); or

includes TTCCCTTTC, preferably includes the sequence TTCCCTTTC linked to the sequence CTTTCCCTT, or more preferably includes the sequence TTCCCTTTC linked to the sequence CTTTCCCTTGTATCTTTT (SEQ ID NO:42).

In some preferred embodiments, the triplex-forming nucleic acid is a peptide nucleic acid including the sequence TJTJJTTT, linked to the sequence TTTCCTCT or TTTCCTCTATGGGTAAG (SEQ ID NO:46); or

TJTTJTJJ linked to the sequence CCTCTTCT, or CCTCTTCTAGTTGGCAT (SEQ ID NO:41); or

TTJJJTTTJ linked to the sequence CTTTCCCTT, or CTTTCCCTTGTATCTTTT (SEQ ID NO:42);

optionally, but preferably wherein one or more of the PNA residues is a γPNA.

In specific embodiments the triplex-forming nucleic acid is a peptide nucleic acid including the sequence is

(SEQ ID NO: 79)
lys-lys-lys-TJTJJTTT-OOO-
TTTCCTCTATGGGTAAG-lys-lys-lys (hCFPNA2);
or
(SEQ ID NO: 79)
lys-lys-lys-TJTJJTTT-OOO-TTTCCTCTATGGGTAAG-lys-lys-
lys;
or
(SEQ ID NO: 80)
lys-lys-lys-TJTTJTJJ-OOO-CCTCTTCTAGTTGGCAT-lys-lys-
lys (hCFPNA1);
or
(SEQ ID NO: 81)
lys-lys-lys-TTJJJTTTJ-OOO-CTTTCCCTTGTATCTTTT-lys-
lys-lys (hCFPNA3);

optionally, but preferably wherein one or more of the PNA residues is a γPNA. In even more specific embodiments, the bolded and underlined residues are miniPEG-containing γPNA.

In some embodiments, a donor that can be used for CFTR gene correction, particularly in combination with the foregoing triplex-forming molecules, includes the sequence 5′TTCTGTATCTATATTCATCATAGGAAACACCAAAGATAATGTTCT CCTTAATGGTGCCAGG3′ (SEQ ID NO:97), or a functional fragment thereof that is suitable and sufficient to correct the F508del mutation in the cystic fibrosis transmembrane conductance regulator (CFTR) gene.

ii. W1282 Mutation Site

In some embodiments, the triplex-forming molecule includes the nucleic acid sequence CTTCCTCTTT (SEQ ID NO:47), preferably includes the sequence CTTCCTCTTT (SEQ ID NO:47) linked to the sequence TTTCTCCTTC (SEQ ID NO:48), or more preferably includes the sequence CTTCCTCTTT (SEQ ID NO:47) linked to the sequence TTTCTCCTTCAGTGTTCA (SEQ ID NO:49); or

the triplex-forming molecule includes the nucleic acid sequence TTTTCCT, preferably includes the sequence TTTTCCT linked to the sequence TCCTTTT, or more preferably includes the sequence TTTTCCT linked to the sequence TCCTTTTGCTCACCTGTGGT (SEQ ID NO:50); or

the triplex-forming molecule includes the nucleic acid sequence TCTTTTTTCC (SEQ ID NO:51), preferably includes the sequence TCTTTTTTCC (SEQ ID NO:51) linked to the sequence CCTTTTTTCT (SEQ ID NO:52), or more preferably includes the sequence TCTTTTTTCC (SEQ ID NO:51) linked to the sequence CCTTTTTTCTGGCTAAGT (SEQ ID NO:53).

In preferred embodiments, the triple forming nucleic acid is a peptide nucleic acid including the sequence JTTJJTJTTT (SEQ ID NO:54) linked to the sequence TTTCTCCTTC (SEQ ID NO:48) or TTTCTCCTTCAGTGTTCA (SEQ ID NO:49); or

a peptide nucleic acid including the sequence TTTTJJT linked to the sequence TCCTTTT or linked to the sequence TCCTTTTGCTCACCTGTGGT (SEQ ID NO:50); or

a peptide nucleic acid including the sequence TJTTTTTTJJ (SEQ ID NO:109) linked to the sequence CCTTTTTTCT (SEQ ID NO:52) or linked to the sequence CCTTTTTTCTGGCTAAGT (SEQ ID NO:53);

optionally, but preferably wherein one or more of the PNA residues is a γPNA.

In specific embodiments, the triplex-forming nucleic acid is a peptide nucleic acid including the sequence

(SEQ ID NO: 82)
lys-lys-lys-JTTJJTJTTT-OOO-
TTTCTCCTTCAGTGTTCA- lys-lys-lys (tcPNA-1236);
or
(SEQ ID NO: 83)
lys-lys-lys-TTTTJJT-OOO-TCCTTTTGCTCACCTGTGGT- lys-
lys-lys (tcPNA-1314);
or
(SEQ ID NO: 84)
lys-lys-lys-TJTTTTTTJJ-OOO-CCTTTTTTCTGGCTAAGT-lys-
lys-lys (tcPNA-1329);

optionally, but preferably wherein one or more of the PNA residues is a γPNA. In even more specific embodiments, the bolded and underlined residues are miniPEG-containing γPNA.

In some embodiments, a donor that can be used for CFTR gene correction, particularly in combination with the foregoing triplex-forming molecules, includes the sequence T(s)C(s)T(s)TGGGATTCAATAACCTTGCAGACAGTGGAGGAAGGCC TTTGGCGTGATACCACAG(s) G(s)T(s)G (SEQ ID NO:98) or a functional fragment thereof that is suitable and sufficient to correct a mutation in CFTR, wherein the bolded and underlined nucleotides are inserted mutations for gene correction, and “(s)” indicates an optional phosphorothiate internucleoside linkage.

iii. G542X Mutation Site

In some embodiments, the triplex-forming molecule includes the nucleic acid sequence TCTTTTT, preferably includes the sequence TCTTTTT linked to the sequence TTTTTCT, or more preferably includes the sequence TCTTTTT linked to the sequence TTTTTCTGTAATTTTTAA (SEQ ID NO:55); or

the triplex-forming molecule includes the nucleic acid sequence TCTCTTTCT, preferably includes the sequence TCTCTTTCT linked to the sequence TCTTTCTCT, or more preferably includes the sequence TCTCTTTCT linked to the sequence TCTTTCTCTGCAAACTT (SEQ ID NO:56); or

the triplex-forming molecule includes the nucleic acid sequence TTTCTTT, preferably includes the sequence TTTCTTT linked to the sequence TTTCTTT, or more preferably includes the sequence TTTCTTT linked to the sequence TTTCTTTAAGAACGAGCA (SEQ ID NO:57).

In preferred embodiments, the triple forming nucleic acid is a peptide nucleic acid including the sequence TJTTTTT linked to the sequence TTTTTCT or TTTTTCTGTAATTTTTAA (SEQ ID NO:55); or

a peptide nucleic acid including the sequence TJTJTTTJT linked to the sequence TCTTTCTCT or linked to the sequence TCTTTCTCTGCAAACTT (SEQ ID NO:56); or

a peptide nucleic acid including the sequence TTTJTTT linked to the sequence TTTCTTT or linked to the sequence TTTCTTTAAGAACGAGCA (SEQ ID NO:57);

optionally, but preferably wherein one or more of the PNA residues is a γPNA.

In specific embodiments, the triplex-forming nucleic acid is a peptide nucleic acid including the sequence

(SEQ ID NO: 85)
lys-lys-lys-TJTTTTT-OOO-
TTTTTCTGTAATTTTTAA-lys-lys-lys (tcPNA-302);
or
(SEQ ID NO: 86)
lys-lys-lys-TJTJTTTJT-OOO-TCTTTCTCTGCAAACTT-lys-
lys-lys (tcPNA-529);
or
(SEQ ID NO: 87)
lys-lys-lys-TTTJTTT-OOO-TTTCTTTAAGAACGAGCA-lys-
lys-lys (tcPNA-586);

optionally, but preferably wherein one or more of the PNA residues is a γPNA. In even more specific embodiments, the bolded and underlined residues are miniPEG-containing γPNA.

In some embodiments, a donor that can be used for CFTR gene correction, particularly in combination with the foregoing triplex-forming molecules, includes the sequence T(s)C(s)C(s)AAGTTTGCAGAGAAAGATAATATAGTCCTTGGAGAAG GAGGAATCACCCTGAGTGGA(s) G(s)G(s)T (SEQ ID NO:99), or a functional fragment thereof that is suitable and sufficient to correct a mutation in CFTR, wherein the bolded and underlined nucleotides are inserted mutations for gene correction, and “(s)” indicates an optional phosphorothiate internucleoside linkage.

Another exemplary donor oligonucleotide is

(SEQ ID NO: 100)
5′-T(s)T(s)G(s)CCCCACAGGGCAGTAACGGCAGACTTCTCCTCAGG
AGTCAGGTGCACCATGGTGTCTGT(s)T(s)T(s)G-3′,

wherein the bolded and underlined residue is the correction and “(s)” indicates a phosphorothiate internucleoside linkage.

In an additional example, (5′-KKK-JTTTTJJJ-OOO-CCCTTTTCAAGGTGAGTAG-KKK-3′) (SEQ ID NO:88) binds in the murine CFTR gene and can be used in combination with donor DNA (5′-TCTTATATCTGTACTCATCATAGGAAACACCAAAGATAATGTTCT CCTTGATAGTACCCGG-3′) (SEQ ID NO:101).

3. HIV

The gene editing compositions can be used to treat or prevent infections, for example those caused by HIV.

a. Exemplary Target Sites

The target sequence for the triplex-forming molecules is within or adjacent to a human gene that encodes a cell surface receptor for human immunodeficiency virus (HIV). Preferably, the target sequence of the triplex-forming molecules is within or is adjacent to a portion of a HIV receptor gene important to its function in HIV entry into cells, such as sequences that are involved in efficient expression of the receptor, transport of the receptor to the cell surface, stability of the receptor, viral binding by the receptor, or endocytosis of the receptor. Target sequences can be within the coding DNA sequence of the gene or within introns. Target sequences can also be within DNA sequences that regulate expression of the target gene, including promoter or enhancer sequences.

The target sequence can be within or adjacent to any gene encoding a cell surface receptor that facilitates entry of HIV into cells. The molecular mechanism of HIV entry into cells involves specific interactions between the viral envelope glycoproteins (env) and two target cell proteins, CD4 and the chemokine receptors. HIV cell tropism is determined by the specificity of the env for a particular chemokine receptor, a 7 transmembrane-spanning, G protein-coupled receptor (Steinberger, et al., Proc. Natl. Acad. Sci. USA. 97: 805-10 (2000)). The two major families of chemokine receptors are the CXC chemokine receptors and the CC chemokine receptors (CCR) so named for their binding of CXC and CC chemokines, respectively. While CXC chemokine receptors traditionally have been associated with acute inflammatory responses, the CCRs are mostly expressed on cell types found in connection with chronic inflammation and T-cell-mediated inflammatory reactions: eosinophils, basophils, monocytes, macrophages, dendritic cells, and T cells (Nansen, et al. 2002, Blood 99:4). In one embodiment, the target sequence is within or adjacent to the human genes encoding chemokine receptors, including, but not limited to, CXCR4, CCR5, CCR2b, CCR3, and CCR1.

In a preferred embodiment, the target sequence is within or adjacent to the human CCR5 gene. The CCR5 chemokine receptor is the major co-receptor for RS-tropic HIV strains, which are responsible for most cases of initial, acute HIV infection. Individuals who possess a homozygous inactivating mutation, referred to as the Δ32 mutation, in the CCR5 gene are almost completely resistant to infection by RS-tropic HIV-1 strains. The Δ32 mutation produces a 32 base pair deletion in the CCR5 coding region.

Another naturally occurring mutation in the CCR5 gene is the m303 mutation, characterized by an open reading frame single T to A base pair transversion at nucleotide 303 which indicates a cysteine to stop codon change in the first extracellular loop of the chemokine receptor protein at amino acid 101 (C101X) (Carrington et al. 1997). Mutagenesis assays have not detected the expression of the m303 co-receptor on the surface of CCR5 null transfected cells which were found to be non-susceptible to HIV-1 R5-isolates in infection assays (Blanpain, et al. (2000).

Compositions and methods for targeted gene therapy using triplex-forming oligonucleotides and peptide nucleic acids for treating infectious diseases such as HIV are described in U.S. Application No. 2008/050920 and WO 2011/133803. Each provides sequences of triplex-forming molecules, target sequences, and donor oligonucleotides that can be utilized in the compositions and methods provided herein.

For example, individuals having the homozygous M2 inactivating mutation in the CCR5 gene display no significant adverse phenotypes, suggesting that this gene is largely dispensable for normal human health. This makes the CCR5 gene a particularly attractive target for targeted mutagenesis using the triplex-forming molecules disclosed herein. The gene for human CCR5 is known in the art and is provided at GENBANK accession number NM_000579. The coding region of the human CCR5 gene is provided by nucleotides 358 to 1416 of GENBANK accession number NM_000579.

In some embodiments, the target region is a polypurine site within or adjacent to a gene encoding a chemokine receptor including CXCR4, CCR5, CCR2b, CCR5, and CCR1. In a preferred embodiment, the target region is a polypurine or homopurine site within the coding region of the human CCR5 gene. Three homopurine sites in the coding region of the CCR5 gene that are especially useful as target sites for triplex-forming molecules are from positions 509-518, 679-690 and 900-908 relative to the ATG start codon. The homopurine site from 679-690 partially encompasses the site of the nonsense mutation created by the M2 mutation. Triplex-forming molecules that bind to this target site are particularly useful.

b. Exemplary Triplex-Forming Sequences

In some embodiments, the triplex-forming molecule includes the nucleic acid sequence CTCTTCTTCT (SEQ ID NO:58), preferably includes the sequence CTCTTCTTCT (SEQ ID NO:58) linked to the sequence TCTTCTTCTC (SEQ ID NO:59), or more preferably includes the sequence CTCTTCTTCT (SEQ ID NO:58) linked to the sequence TCTTCTTCTCATTTC (SEQ ID NO:60).

In some embodiments, the triplex-forming molecule includes the nucleic acid sequence CTTCT, preferably includes the sequence CTTCT linked to the sequence TCTTC or TCTTCTTCTC (SEQ ID NO:59), or more preferably includes the sequence CTTCT linked to the sequence TCTTCTTCTCATTTC (SEQ ID NO:60).

In preferred embodiments, the triplex-forming nucleic acid is a peptide nucleic acid including the sequence JTJTTJTTJT (SEQ ID NO:110) linked to the sequence TCTTCTTCTC (SEQ ID NO:59) or TCTTCTTCTCATTTC (SEQ ID NO:60);

or JTTJT linked to the sequence TCTTC or TCTTCTTCTC (SEQ ID NO:59) or more preferably TCTTCTTCTCATTTC (SEQ ID NO:60);

optionally, but preferably wherein one or more of the PNA residues is a γPNA.

In specific embodiments, the triplex-forming nucleic acid is a peptide nucleic acid including the sequence Lys-Lys-Lys-JTJTTJTTJT-OOO-TCTTCTTCTCATTTC-Lys-Lys-Lys (SEQ ID NO:89) (PNA-679);

or Lys-Lys-Lys-JTTJT-OOO-TCTTCTTCTCATTTC-Lys-Lys-Lys (SEQ ID NO:90) (tcPNA-684) optionally, but preferably wherein one or more of the PNA residues is a γPNA. In even more specific embodiments, the bolded and underlined residues are miniPEG-containing γPNA.

c. Exemplary Donor Sequences

In some embodiments, the triplex-forming molecules are used in combination with one or more donor oligonucleotides such as donor 591 having the sequence: 5′ AT TCC CGA GTA GCA GAT GAC CAT GAC AGC TTA GGG CAG GAC CAG CCC CAA GAT GAC TAT C 3′ (SEQ ID NO:102), or donor 597 having the sequence 5′ TT TAG GAT TCC CGA GTA GCA GAT GAC CCC TCA GAG CAG CGG CAG GAC CAG CCC CAA GAT G 3′ (SEQ ID NO:103), which can be used in combination to induce two different non-sense mutations, one in each allele of the CCR5 gene, in the vicinity of the Δ32 deletion (mutation sites are bolded); or a functional fragment thereof that is suitable and sufficient to introduce a non-sense mutation in at least one allele of the CCR5 gene.

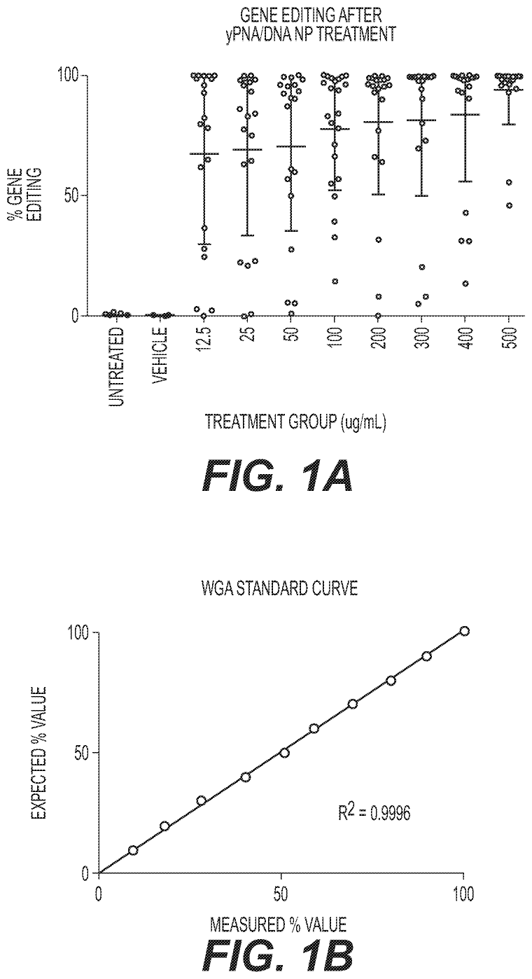

In another preferred embodiment, donor oligonucleotides are designed to span the Δ32 deletion site (see, e.g., FIG. 1 of WO 2011/133803) and induce changes into a wildtype CCR5 allele that mimic the Δ32 deletion. Donor sequences designed to target the Δ32 deletion site may be particularly usefully to facilitate knockout of the single wildtype CCR5 allele in heterozygous cells.

Preferred donor sequences designed to target the Δ32 deletion site include, but are not limited to,

Donor DELTA32JDC:
(SEQ ID NO: 104)
5′GATGACTATCTTTAATGTCTGGAAATTCTTCCAGAATTAA
TTAAGACTGTATGGAAAATGAGAGC 3′;
Donor DELTAJDC2:
(SEQ ID NO: 105)
5′CCCCAAGATGACTATCTTTAATGTCTGGAACGATCATCAG
AATTGATACTGACTGTATGGAAAATG 3′;
and
Donor DELTA32RSB:
(SEQ ID NO: 106)
5′GATGACTATCTTTAATGTCTGGAAATTCTACTAGAATTGA
TACTGACTGTATGGAAAATGAGAGC 3′,

or a functional fragment of SEQ ID NO:104, 105, or 106 that is suitable and sufficient to introduce mutation CCR5 gene.

4. Lysosomal Storage Diseases

The compositions and methods compositions can also be used to treat lysosomal storage diseases. Lysosomal storage diseases (LSDs) are a group of more than 50 clinically-recognized, rare inherited metabolic disorders that result from defects in lysosomal function (Walkley, J. Inherit. Metab. Dis., 32(2):181-9 (2009)). Lysosomal storage disorders are caused by dysfunction of the cell's lysosome orangelle, which is part of the larger endosomal/lysosomal system. Together with the ubiquitin-proteosomal and autophagosomal systems, the lysosome is essential to substrate degradation and recycling, homeostatic control, and signaling within the cell. Lysosomal dysfunction is usually the result of a deficiency of a single enzyme necessary for the metabolism of lipids, glycoproteins (sugar containing proteins) or mucopolysaccharides (long unbranched polysaccharides consisting of a repeating disaccharide unit; also known as glycosaminoglycans, or GAGs) which are fated for breakdown or recycling. Enzyme deficiency reduces or prevents break down or recycling of the unwanted lipids, glycoproteins, and GAGs, and results in buildup or “storage” of these materials within the cell. Most lysosomal diseases show widespread tissue and organ involvement, with brain, viscera, bone and connective tissues often being affected. More than two-thirds of lysosomal diseases affect the brain. Neurons appear particularly vulnerable to lysosomal dysfunction, exhibiting a range of defects from specific axonal and dendritic abnormalities to neuron death.

Individually, LSDs occur with incidences of less than 1:100,000, however, as a group the incidence is as high as 1 in 1,500 to 7,000 live births (Staretz-Chacham, et al., Pediatrics, 123(4):1191-207 (2009)). LSDs are typically the result of inborn genetic errors. Most of these disorders are autosomal recessively inherited, however a few are X-linked recessively inherited, such as Fabry disease and Hunter syndrome (MPS II). Affected individuals generally appear normal at birth, however the diseases are progressive. Develop of clinical disease may not occur until years or decades later, but is typically fatal. Lysosomal storage diseases affect mostly children and they often die at a young and unpredictable age, many within a few months or years of birth. This makes these types of lysosomal storage diseases attractive for pre-natal intervention. Many other children die of this disease following years of suffering from various symptoms of their particular disorder. Clinical disease may be manifest as mental retardation and/or dementia, sensory loss including blindness or deafness, motor system dysfunction, seizures, sleep and behavioral disturbances, and so forth. Some people with Lysosomal storage disease have enlarged livers (hepatomegaly) and enlarged spleens (splenomegaly), pulmonary and cardiac problems, and bones that grow abnormally.

Treatment for many LSDs is enzyme replacement therapy (ERT) and/or substrate reduction therapy (SRT), as wells as treatment or management of symptoms. The average annual cost of ERT in the United States ranges from $90,000 to $565,000. While ERT has significant systemic clinical efficacy for a variety of LSDs, little or no effects are seen on central nervous system (CNS) disease symptoms, because the recombinant proteins cannot penetrate the blood-brain barrier. Allogeneic hematopoietic stem cell transplantation (HSCT) represents a highly effective treatment for selected LSDs. It is currently the only means to prevent the progression of associated neurologic sequelae. However, HSCT is expensive, requires an HLA-matched donor and is associated with significant morbidity and mortality. Recent gene therapy studies suggest that LSDs are good targets for this type of treatment.

Compositions and methods for targeted gene therapy using triplex-forming oligonucleotides and peptide nucleic acids for treating lysosomal storage diseases are described in WO 2011/133802, which provides sequences of triplex-forming molecules, target sequences, and donor oligonucleotides that can be utilized in the compositions and methods provided herein.

For example, the compositions and methods can be are employed to treat Gaucher's disease (GD). Gaucher's disease, also known as Gaucher syndrome, is the most common lysosomal storage disease. Gaucher's disease is an inherited genetic disease in which lipid accumulates in cells and certain organs due to deficiency of the enzyme glucocerebrosidase (also known as acid β-glucosidase) in lysosomes. Glucocerebrosidase enzyme contributes to the degradation of the fatty substance glucocerebroside (also known as glucosylceramide) by cleaving b-glycoside into b-glucose and ceramide residues (Scriver C R, Beaudet A L, Valle D, Sly W S. The metabolic and molecular basis of inherited disease. 8th ed. New York: McGraw-Hill Pub, 2001: 3635-3668). When the enzyme is defective, the substance accumulates, particularly in cells of the mononuclear cell lineage, and organs and tissues including the spleen, liver, kidneys, lungs, brain and bone marrow.

There are two major forms: non-neuropathic (type 1, most commonly observed type in adulthood) and neuropathic (type 2 and 3). GBA (GBA glucosidase, beta, acid), the only known human gene responsible for glucosidase-mediated GD, is located on chromosome 1, location 1q21. More than 200 mutations have been defined within the known genomic sequence of this single gene (NCBI Reference Sequence: NG_009783.1). The most commonly observed mutations are N370S, L444P, RecNciI, 84GG, R463C, recTL and 84 GG is a null mutation in which there is no capacity to synthesize enzyme. However, N370S mutation is almost always related with type 1 disease and milder forms of disease. Very rarely, deficiency of sphingolipid activator protein (Gaucher factor, SAP-2, saposin C) may result in GD. In some embodiments, triplex-forming molecules are used to induce recombination of donor oligonucleotides designed to correct mutations in GBA.

In another embodiment, compositions and the methods herein are used to treat Fabry disease (also known as Fabry's disease, Anderson-Fabry disease, angiokeratoma corporis diffusum and alpha-galactosidase A deficiency), a rare X-linked recessive disordered, resulting from a deficiency of the enzyme alpha galactosidase A (a-GAL A, encoded by GLA). The human gene encoding GLA has a known genomic sequence (NCBI Reference Sequence: NG_007119.1) and is located at Xp22 of the X chromosome. Mutations in GLA result in accumulation of the glycolipid globotriaosylceramide (abbreviated as Gb3, GL-3, or ceramide trihexoside) within the blood vessels, other tissues, and organs, resulting in impairment of their proper function (Karen, et al., Dermatol. Online J., 11 (4): 8 (2005)). The condition affects hemizygous males (i.e. all males), as well as homozygous, and potentially heterozygous (carrier), females. Males typically experience severe symptoms, while women can range from being asymptomatic to having severe symptoms. This variability is thought to be due to X-inactivation patterns during embryonic development of the female. In some embodiments, triplex-forming molecules are used to induce recombination of donor oligonucleotides designed to correct mutations in GLA.

In preferred embodiments, the compositions and methods are used to treat Hurler syndrome (HS). Hurler syndrome, also known as mucopolysaccharidosis type I (MPS I), α-L-iduronidase deficiency, and Hurler's disease, is a genetic disorder that results in the buildup of mucopolysaccharides due to a deficiency of α-L iduronidase, an enzyme responsible for the degradation of mucopolysaccharides in lysosomes (Dib and Pastories, Genet. Mol. Res., 6(3):667-74 (2007)). MPS I is divided into three subtypes based on severity of symptoms. All three types result from an absence of, or insufficient levels of, the enzyme α-L-iduronidase. MPS I H or Hurler syndrome is the most severe of the MPS I subtypes. The other two types are MPS I S or Scheie syndrome and MPS I H-S or Hurler-Scheie syndrome. Without α-L-iduronidase, heparan sulfate and dermatan sulfate, the main components of connective tissues, build-up in the body. Excessive amounts of glycosaminoglycans (GAGs) pass into the blood circulation and are stored throughout the body, with some excreted in the urine. Symptoms appear during childhood, and can include developmental delay as early as the first year of age. Patients usually reach a plateau in their development between the ages of two and four years, followed by progressive mental decline and loss of physical skills (Scott et al., Hum. Mutat. 6: 288-302 (1995)). Language may be limited due to hearing loss and an enlarged tongue, and eventually sight impairment can result from clouding of cornea and retinal degeneration. Carpal tunnel syndrome (or similar compression of nerves elsewhere in the body) and restricted joint movement are also common.

a. Exemplary Target Sites

The human gene encoding alpha-L-iduronidase (α-L-iduronidase; IDUA) is found on chromosome 4, location 4p16.3, and has a known genomic sequence (NCBI Reference Sequence: NG_008103.1). Two of the most common mutations in IDUA contributing to Hurler syndrome are the Q70X and the W420X, non-sense point mutations found in exon 2 (nucleotide 774 of genomic DNA relative to first nucleotide of start codon) and exon 9 (nucleotide 15663 of genomic DNA relative to first nucleotide of start codon) of IDUA respectively. These mutations cause dysfunction alpha-L-iduronidase enzyme. Two triplex-forming molecule target sequences including a polypurine:polypyrimidine stretches have been identified within the IDUA gene. One target site with the polypurine sequence 5′ CTGCTCGGAAGA 3′ (SEQ ID NO:61) and the complementary polypyrimidine sequence 5′ TCTTCCGAGCAG 3′ (SEQ ID NO:62) is located 170 base pairs downstream of the Q70X mutation. A second target site with the polypurine sequence 5′ CCTTCACCAAGGGGA 3′ (SEQ ID NO:63) and the complementary polypyrimidine sequence 5′ TCCCCTTGGTGAAGG 3′ (SEQ ID NO:64) is located 100 base pairs upstream of the W402X mutation. In preferred embodiments, triplex-forming molecules are designed to bind/hybridize in or near these target locations.

b. Exemplary Triplex-Forming Sequences and Donors

i. W402X Mutation

In some embodiments, a triplex-forming molecule binds to the target sequence upstream of the W402X mutation includes the nucleic acid sequence TTCCCCT, preferably includes the sequence TTCCCCT linked to the sequence TCCCCTT, or more preferably includes the sequence TTCCCCT linked to the sequence TCCCCTTGGTGAAGG (SEQ ID NO:64).

In some preferred embodiments, the triplex-forming nucleic acid is a peptide nucleic acid that binds to the target sequence upstream of the W402X mutation including the sequence TTJJJJT, linked to the sequence TCCCCTT or TCCCCTTGGTGAAGG (SEQ ID NO:64), optionally, but preferably wherein one or more of the PNA residues is a γPNA.

In specific embodiments, the triplex-forming nucleic acid is a peptide nucleic acid having the sequence Lys-Lys-Lys-TTJJJJT-OOO-TCCCCTTGGTGAAGG-Lys-Lys-Lys (SEQ ID NO:91) (IDUA402tc715) optionally, but preferably wherein one or more of the PNA residues is a γPNA. In even more specific embodiments, the bolded and underlined residues are miniPEG-containing γPNA.

In the most preferred embodiments, triplex-forming molecules are administered according to the methods in combination with one or more donor oligonucleotides designed to correct the point mutations at Q70X or W402X mutations sites. In some embodiments, in addition to containing sequence designed to correct the point mutation at Q70X or W402X mutation, the donor oligonuclotides may also contain 7 to 10 additional, synonymous (silent) mutations. The additional silent mutations can facilitate detection of the corrected target sequence using allele-specific PCR of genomic DNA isolated from treated cells.

In some embodiments, the donor oligonucleotide with the sequence 5′ AGGACGGTCCCGGCCTGCGACACTTCCGCCCATAATTGTTCTTCAT CTGCGGGGCGGGGGGGGG 3′ (SEQ ID NO:107), or a functional fragment thereof that is suitable and sufficient to correct the W402X mutation is administered with triplex-forming molecules designed to target the binding site upstream of W402X to correct the W402X mutation in cells.

ii. Q70X Mutation

In some embodiments, a triplex-forming molecule that binds to the target sequence downstream of the Q70X mutation includes the nucleic acid sequence CCTTCT, preferably includes the sequence CCTTCT linked to the sequence TCTTCC, or more preferably includes the sequence CCTTCT linked to the sequence TCTTCCGAGCAG (SEQ ID NO:62).

In preferred embodiments, the triplex-forming nucleic acid is a peptide nucleic acid that binds to the target sequence downstream of the Q70X mutation including the sequence JJTTJT linked to the sequence TCTTCC or TCTTCCGAGCAG (SEQ ID NO:62) optionally, but preferably wherein one or more of the PNA residues is a γPNA.

In a specific embodiment, a tcPNA with a sequence of Lys-Lys-Lys-JJTTJT-OOO-TCTTCCGAGCAG-Lys-Lys-Lys (SEQ ID NO:92) (IDUA402tc715) optionally, but preferably wherein one or more of the PNA residues is a γPNA. In even more specific embodiments, the bolded and underlined residues are miniPEG-containing γPNA.

A donor oligonucleotide can have the sequence 5′GGGACGGCGCCCACATAGGCCAAATTCAATTGCTGATCCCAGCT TAAGACGTACTGGTCAGCCTGGC 3′ (SEQ ID NO:108), or a functional fragment thereof that is suitable and sufficient to correct the Q70X mutation is administered with triplex-forming molecules designed to target the binding site downstream of Q70X to correct the of Q70X mutation in cells.

### XI. Combination Therapies

The compositions can be used in combination with other mutagenic agents. In a preferred embodiment, the additional mutagenic agents are conjugated or linked to gene editing technology or a delivery vehicle (such as a nanoparticle or microparticle) thereof. Additional mutagenic agents that can be used in combination with gene editing technology, particularly triplex-forming molecules, include agents that are capable of directing mutagenesis, nucleic acid crosslinkers, radioactive agents, or alkylating groups, or molecules that can recruit DNA-damaging cellular enzymes. Other suitable mutagenic agents include, but are not limited to, chemical mutagenic agents such as alkylating, bialkylating or intercalating agents. A preferred agent for co-administration is psoralen-linked molecules as described in PCT/US/94/07234 by Yale University.

It may also be desirable to administer gene editing compositions in combination with agents that further enhance the frequency of gene modification in cells. For example, the compositions can be administered in combination with a histone deacetylase (HDAC) inhibitor, such as suberoylanilide hydroxamic acid (SAHA), which has been found to promote increased levels of gene targeting in asynchronous cells.

The nucleotide excision repair pathway is also known to facilitate triplex-forming molecule-mediated recombination. Therefore, the compositions can be administered in combination with an agent that enhances or increases the nucleotide excision repair pathway, for example an agent that increases the expression, or activity, or localization to the target site, of the endogenous damage recognition factor XPA.

Compositions may also be administered in combination with a second active agent that enhances uptake or delivery of the gene editing technology. For example, the lysosomotropic agent chloroquine has been shown to enhance delivery of PNAs into cells (Abes, et al., J. Controll. Rel., 110:595-604 (2006). Agents that improve the frequency of gene modification are particularly useful for in vitro and ex vivo application, for example ex vivo modification of hematopoietic stem cells for therapeutic use.

### XII. Methods for Determining Triplex Formation and Gene Modification

A. Methods for Determining Triplex Formation

A useful measure of triple helix formation is the equilibrium dissociation constant, K_{d}, of the triplex, which can be estimated as the concentration of triplex-forming molecules at which triplex formation is half-maximal. Preferably, the molecules have a binding affinity for the target sequence in the range of physiologic interactions. Preferred triplex-forming molecules have a K_{d} less than or equal to approximately 10⁻⁷ M. Most preferably, the K_{d} is less than or equal to 2×10⁻⁸ M in order to achieve significant intramolecular interactions. A variety of methods are available to determine the K_{d} of triplex-forming molecules with the target duplex. In the examples which follow, the K_{d} was estimated using a gel mobility shift assay (R. H. Durland et al., Biochemistry 30, 9246 (1991)). The dissociation constant (K_{d}) can be determined as the concentration of triplex-forming molecules in which half was bound to the target sequence and half was unbound.

B. Methods for Determining Gene Modification

Sequencing, allele-specific PCR, and droplet digital PCR, are preferred methods for determining if gene modification has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, droplet digital PCR (ddPCR), or restriction endonuclease selective PCR (REMS-PCR); analysis of mRNA transcribed from the target gene for example by Northern blot, in situ hybridization, real-time or quantitative reverse transcriptase (RT) PCT; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

### XIII. Kits

Medical kits are also disclosed. The medical kits can include, for example, a dosage supply of gene editing technology active agent(s) optionally particle-loaded, a potentiating agent thereof, or a combination thereof separately or together in the same admixture. The active agents can be supplied alone (e.g., lyophilized), or in a pharmaceutical composition. The active agents can be in a unit dosage, or in a stock that should be diluted prior to administration. In some embodiments, the kit includes a supply of pharmaceutically acceptable carrier. The kit can also include devices for administration of the active agents or compositions, for example, syringes. The kits can include printed instructions for administering the compound in a use as described above.

### EXAMPLES

### Example 1: Synthetic Nanoparticle Delivery to Mouse Embryos for Site-Specific Genome Editing

The following experiments exemplify the use of synthetic nanoparticles to deliver peptide nucleic acids and donor DNA to achieve site-specific non-enzymatic gene editing in murine embryos. The approach uses nanoparticles fabricated from a biodegradable polymer, poly(lactic-co-glycolic acid) (PLGA), which has been used in humans for over 40 years and is well known to be safe. To achieve gene correction, two active agents are loaded into the nanoparticles: a triplex-forming peptide nucleic acid (PNA) that induces recombination at a specific, targeted site on a chromosome and a short donor DNA molecule that contains the desired gene sequence. Once administered, PLGA nanoparticles (NPs) are taken up by cells where they degrade, releasing the loaded PNA and DNA. Once inside a cell, the engineered PNA binds to a specific genomic target site, forming a triplex, which induces DNA repair mechanisms that stimulate the recombination of the short, single stranded donor DNA molecule containing the correct sequence, resulting in site-specific gene correction. The genetic corrections achieved after nanoparticle delivery are permanent and heritable; genes corrected in stem cells are passed down to progeny cells as they differentiate.

### Materials and Methods

Mouse Model

All animal use was in accordance with the guidelines of the Animal Care and Use Committee of Yale University and conformed to the recommendations in the Guide for the Care and Use of Laboratory Animals (Institute of Laboratory Animal Resources, National Research Council, National Academy of Sciences, 1996). IVS2-654 β-thalassemia mice were used (Lewis et al., Blood, 91(6): p. 2152-6 (1998)).

Superovulation and Embryo Harvest

To obtain single cell fertilized embryos, 5-8 week old female mice are superovulated by intraperitoneal (IP) injection with 5 IU pregnant mare's serum gonadotropin (PMSG). After 48 hours, 5 IU of human chorionic gonadotropin (HCG) is administered via IP injection. The hormone treated females are mated with stud mice overnight and examined for sperm plugs the next morning; plugged females are euthanized. Their ovaries are dissected and are placed into a petri dish containing light mineral oil and a 50 μl droplet of hyaluronidase solution (Irvine Scientific #90101) and three 50 μl droplets of Embryomax M2 media (Sigma, #M7167). The ampulla of the oviduct is torn using a 31G needle to release the fertilized embryos. Harvested embryos are immediately transferred to a drop of hyaluronidase solution to digest off the cumulus cells for thirty seconds. Embryos are washed with fresh hyaluronidase-free M2 media three times and then transferred to 72-well 10 μl tissue culture dish (1-4 embryos per well) containing KSOM media (EMD Millipore, #MR-106-D) pre-equilibrated for an hour in a 37° C. incubator with 5% CO2. The 72 well plate containing embryos is covered in 7 ml of light mineral oil (Sigma, #M5310) prior to transfer to the 37° C. incubator (Behringer, New York: Cold Spring Harbor Laboratory Press. xxii, 814 pages, Gilbert et al., Reprod Biol Endocrinol, 14: p. 13 (2016)).

Oligonucleotides

Mini-PEG γPNA monomers were prepared from Boc-(2-(2-methoxyethoxy)ethyl)-L-serine as a starting material by a series of multistep synthetic procedures including reduction, mitsunobu reaction, nucleobase (A, C, G and T) conjugation and then ester cleavage (Bahal et al., Chembiochem, 13(1): p. 56-60 (2012)). At each step the respective product was purified by column chromatography (Bahal et al., Chembiochem, 13(1): p. 56-60 (2012)) PNA oligomers were synthesized on solid support using Boc chemistry (Bahal et al., Chembiochem, 13(1): p. 56-60 (2012)). The oligomers were synthesized on MBHA (4-methylbenzhydrylamine) resin according to standard procedures of Boc chemistry. A kaiser test was performed at each step to measure complete coupling and double coupling was performed if it was required. The oligomers were cleaved from the resin using an m-cresol/thioanisole/TFMSA/TFA (1:1:2:6) cocktail, and the resulting mixtures were precipitated with ethyl ether, purified by reversed phase-high-performance liquid chromatography (acetonitrile:water) and characterized with a matrix-assisted laser desorption/ionization time-of-flight mass spectrometer (Bahal et al., Chembiochem, 13(1): p. 56-60 (2012)). The sequence of γPNA used in this study is H-KKK-JTTTJTTTJTJT-OOO-TCTCTTTCTTTCAGGGCA-KKK-NH₂ (SEQ ID NO:66). Underlined indicates γPNA residues; K, lysine; J, pseudoisocytosine; 0, 8-amino-2,6,10-trioxaoctanoic acid linkers connecting the Hoogsteen and Watson-Crick domains of the tcPNA. During PNA synthesis, the fluorescent dye 5-Carboxytetramethylrhodamine (TAMRA; Biotium) was conjugated to the N-terminus of a fraction of the synthesized PNA prior to cleavage with m-cresol/thioanisole/TFMSA/TFA. The single-stranded donor DNA oligomer was prepared by standard DNA synthesis except for the inclusion of three phosphorothioate internucleoside linkages at each end to protect against nuclease degradation. The 60 bp donor DNA matches positions 624-684 in β-globin intron 2, with the correcting IVS2-654 nucleotide underlined:

(SEQ ID NO: 93)
5′AAAGAATAACAGTGATAATTTCTGGGTTAAGGCAATAGCAATA
TCTCTGCATATAAATAT3′.

Nanoparticle Fabrication

The PLGA used for these studies is as follows: 50:50 ester-terminated, 0.95-1.2 g dl⁻¹ (LACTEL absorbable polymers; Birmingham, Ala.). NPs containing C6 (Sigma; St Louis, Mo.) were formulated using a single-emulsion solvent evaporation technique (McNeer et al., Mol Ther, 19(1): p. 172-80 (2011)). C6 was added to the polymer solution at a 0.2% wt:wt dye:polymer ratio. C6 and 80 mg of PLGA were dissolved in 800 μl dichloromethane (DCM) overnight. This mixture was added dropwise to 1.6 mL 5% aqueous polyvinyl alcohol (PVA), then ultrasonicated (3×10 s) to formulate a single emulsion. This mixture was poured into 20 ml of 0.3% aqueous PVA and stirred for 3 hours at room temperature. Nanoparticles were then thoroughly washed with 20 ml water (3×) and further collected each time by centrifugation (25,644×g for 10 min at 4° C.). Nanoparticles were resuspended in water, frozen at −80° C., and then lyophilized. Nanoparticles were stored at −20° C. after lyophilization (Bahal et al., Nat Commun, 7: p. 13304 (2016)).

PNA/DNA PLGA NPs were formulated using a double-emulsion solvent evaporation technique modified to encapsulate PNA and DNA oligomers (Bahal et al., Nat Commun, 7: p. 13304 (2016), Ricciardi et al., Methods Mol Biol, 1176: p. 89-106 (2014)). PNAs and donor DNAs were dissolved in 60.8 μl DNAse-free water. All nanoparticle batches had 2 nmole mg⁻¹ of γPNA and 1 nmole mg⁻¹ of donor DNA. The encapsulant was added dropwise to a polymer solution containing 80 mg 50:50 ester-terminated PLGA dissolved in dichloromethane (800 μl), then ultrasonicated (3×10 s) to formulate the first emulsion. To form the second emulsion, the first emulsion was added slowly dropwise to 1.6 ml of 5% aqueous polyvinyl alcohol and then ultrasonicated (3×10 s). This mixture was finally poured into 20 ml of 0.3% aqueous polyvinyl alcohol and stirred for 3 hours at room temperature. Nanoparticles were then thoroughly washed with 20 ml water (3×) and further collected each time by centrifugation (25,644×g for 10 min at 4° C.). Nanoparticles were resuspended in water, frozen at −80° C., and then lyophilized Nanoparticles were stored at −20° C. after lyophilization (Bahal et al., Nat Commun, 7: p. 13304 (2016)). Dynamic light scattering (DLS) was performed to measure the NPs size (hydrodynamic diameter) and surface charge (zeta potential) using a Malvern Nano-ZS (Malvern Instruments, UK).

Embryo Treatment

Embryos are allowed to equilibrate in the KSOM medium within the cell incubator for an hour prior to NP treatment. NPs are resuspended in KSOM prior to NP administration. Embryos were treated with a range of NP concentrations from 0-500 μg ml⁻¹. One hour after NP treatment, embryos were washed three times and then transferred to a well containing fresh KSOM medium. Prior to imaging, some embryos were stained with Hoescht 33342 to visualize the nuclei. Imaging of live embryos was performed using an EVOS FL Cell Imaging System (Thermo Fischer Scientific, Waltham, Mass., USA).

Isolation of gDNA from Blastocysts

Blastocyst lysis buffer is made as follows for 10 mL: 500 μl 1M Tris, 20 μl 0.5M EDTA, 500 μl of 10% Tween-20, and 9 ml dH₂O. Blastocyst lysis buffer has the final concentration: 50 mM Tris pH 8.8, 1 mM EDTA pH 8.0, and 0.5% Tween-20. Proteinase K is added prior to use: 1.5 μl of proteinase K (20 mg ml⁻¹) in 50 μl of lysis buffer. One blastocyst is incubated in 10 μl of lysis buffer at 55° C. overnight. In the morning the sample is heat inactivated at 95° C. for 15 minutes and then cooled to room temperature prior to use.

Whole Genome Amplification

2.5 μl of gDNA from the blastocyst digestion as described above was used for whole genome amplification (Lucigen, Sygnis TruePrime WGA Kit #SYG380100). Kit instructions were as follows: 2.5 μl of buffer D was added to each gDNA sample and allowed to incubate for three minutes to denature the gDNA. 2.5 μl of buffer N was then added to neutralize the denaturation reaction. Amplification mix was then added as follows: 26.8 μl of dH₂O, 5 μl reaction buffer, 5 μl dNTPs, 5 μl enzyme 1, and 0.7 μl enzyme 2. Samples were then incubated at 30° C. for 6 hours then heat inactivated at 65° C. for 10 minutes. Samples were purified using MicroBio-Spin P-30 Tris Chromatography columns (BioRad, Hercules, Calif.). Columns were inverted to resuspend the gel matrix, then centrifuged at 1000×(g) for two minutes to remove the matrix. Columns were placed in 1.5 mL microcentrifuge tubes and 20 μL of WGA sample was applied to the center of the column. Sample was centrifuged for four minutes at 1000×(g). Samples were then placed in −20° C. freezer until further use. To check the fidelity of the WGA, a standard curve was constructed from known amounts of gDNA (Supplementary FIG. 1) and the template appears to amplify with high fidelity (R²=0.9996).

Droplet digital PCR

1-10 μl of the whole genome amplified gDNA or gDNA from reimplanted mice was used for droplet digital PCR. PCR reactions were set up as followed: 11 μl 2× ddPCR™ supermix for probes (no dUTP) (Bio-Rad, Hercules, Calif.), 0.2 μl forward primer (100 μM), 0.2 μl reverse primer (100 μM), 0.053 μl β-thal probe (100 μM), 0.053 μl wild-type probe (100 μM) (Integrated DNA Technologies, Coralville, Iowa), 0.5 μl EcoR1, 10 μl gDNA and dH₂O. Droplets were generated using the Automated Droplet Generator (AutoDG™) (Bio-Rad). Thermocycling conditions were as follows: 95° C. 10 min, (94° C. 30 s, 55.3° C. 5 min−ramp 2° C./s)×40 cycles, 98° C. 10 min, hold at 4° C. Droplets were allowed to rest at 4° C. for at least 30 minutes after cycling and were then read using the QX200™ Droplet Reader (Bio-Rad). Data were analyzed using QuantaSoft™ software. The primers used for ddPCR were as follows: forward: 5′-ACCATTCTAAAGAATAACAGTGA-3′ (SEQ ID NO:113), reverse: 5′-CCTCTTACATCAGTTACAATTT-3′ (SEQ ID NO:114). The probes used for ddPCR were as follows: wild-type (FAM): 5′ TGGGTTAAGGCAATAGCAA 3′ (SEQ ID NO:115), β-thal (HEX): 5′ TCTGGGTTAAGGTAATAGCAAT 3′ (SEQ ID NO:65).

### Results

Mammalian oocytes are protected by a glycoprotein coating called the zona pellucida (ZP). The mouse ZP is a thick extracellular coat with an average diameter of 6.5 μm and an average of 3.5 ng of glycoprotein (Wassarman et al., Cytogenet Genome Res, 105(2-4): p. 228-34 (2004)). The ZP is an extremely porous coat that can be penetrated by large macromolecules, such as antibodies and enzymes, as well as by small viruses (Wassarman et al., Cytogenet Genome Res, 105(2-4): p. 228-34 (2004)). Antibodies are typically 10 nm and most viruses range from 20-200 nm. The PLGA NPs used for delivery have an average hydrodynamic diameter of 250 nm. To investigate if the NPs could penetrate the ZP of fertilized mouse oocytes, NPs loaded with coumarin-6 dye were pipetted into embryo medium. After an hour of NP treatment, all tested doses (12.5-500 μg/mL) resulted in NP accumulation within the embryo, indicating that the 250 nm PLGA NPs can traverse the ZP.

Although NPs can penetrate the ZP, to achieve gene editing, reagents such as PNA and donor DNA must reach the nucleus. One advantage of embryo editing with PNA/DNA NPs is the elimination of the technically challenging pronuclear microinjection step that is used for gene editing with CRISPR/Cas9. Experiments were designed to investigate NP nuclear delivery. To determine if NPs were reaching the nuclei of treated embryos, cells were stained with Hoescht 33342, a nuclear stain that allows for live cell imaging, and imaged one hour after fluorescent NP delivery. Nuclear clearing, or a region of the cell that contains fewer fluorescent NPs and coincides with the nuclear staining, was observed one hour after NP treatment. At the beginning of mitosis the nuclear envelope disassembles, allowing for mixing of the nuclear and cytoplasmic contents. During telophase, the nuclear envelope reforms around the condensed chromosomes at either pole of the cell. Nuclear envelope break-down and reassembly could allow for the re-localization of NPs that were initially in the cytoplasm to the nucleus, and this could potentially allow for high levels of editing even if NPs are not initially in high concentrations within the nucleus.

To examine the delivery of PNA to embryos, PLGA NPs containing fluorescently labeled PNA were fabricated. The size, zeta potential, and loading were similar to NPs loaded with unlabeled oligomers (Table 2).

TABLE 2
PNA/DNA PLGA NP Characterization

| | | | zeta | Loading |
|---|---|---|---|---|
| | | | potential | (OD |
| | z-avg (nm) | PDI | (mV) | mg⁻¹ ml⁻¹) |
| γPNA-TAMRA/DNA- | 238 +/− 3.4 | 0.026 | −24.7 +/− 3 | 0.46 |
| Dylight 488 NPs | | | | |
| γPNA/DNA NPs | 239.4 +/− 4.7 | 0.040 | −25.6 +/− 6 | 0.42 |

PNA was labeled with tetramethylrhodamine(TAMRA). The labeled-oligomer NPs are much dimmer than the fluorescent dye NPs, since the weight percentage of dye in the NPs (0.3%) is much higher than the weight percentage of the labeled oligos in the NPs (˜0.02%).

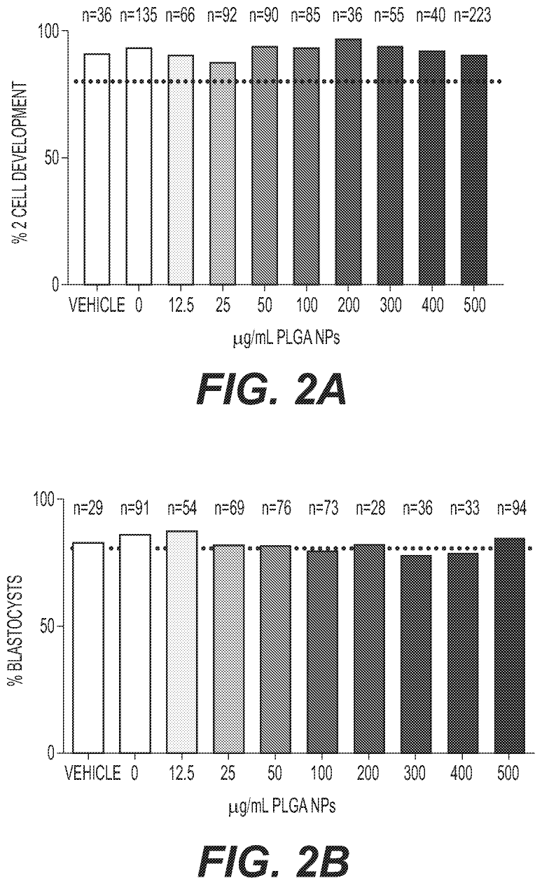

One day after NP treatment, a signal from the TAMRA-PNA can be observed uniformly throughout the treated embryos. After 5 days, a strong signal from the TAMRA-PNA is still detected, indicating that the PNA persists within the embryonic cells throughout multiple cell divisions. NP treatment with a variety of encapsulants, including PNA and DNA, did not impede further cell division. Two measures of embryo health in culture are development to the two-cell stage and blastocyst hatching from the ZP (Nagy et al., Fertil Steril, 80(1): p. 67-74 (2003)). The ZP serves many functions, but during the early stages of embryo development, the ZP prevents the embryo from implanting into the fallopian tube of a human, or the oviduct of a mouse. Once the embryo traverses the oviduct and reaches the uterus, a healthily dividing embryo should hatch from the ZP to allow for implantation into the uterine wall. It was observed that the NP treated embryos hatch from the ZP in culture and that NP treatment does not negatively impact the percentage of embryos reaching the two-cell or blastocyst stage for any of the NP doses tested (FIGS. 2A and 2B).

Single-cell embryos were harvested from a hemizygous 654-eGFP reporter mouse model. Each embryo has one copy of a GFP-beta globin fusion transgene with a IVS2-654 (C to T) mutation causing an aberrantly expressed intron; correction results in expression of a functional GFP mRNA transcript (Lewis et al., Blood, 91(6): p. 2152-6 (1998)). PNA/DNA containing NPs were pipetted onto media containing healthy single-cell fertilized embryos. After one hour of treatment, embryos were washed and transferred to fresh media. Editing was evaluated five days post NP treatment. Genomic DNA was isolated from single blastocysts, when an embryo contains approximately 100 cells. Since the 654-eGFP cells each have one transgene, this results in approximately 100 detectable alleles. To be able to quantify editing with more accuracy, gDNA samples then underwent whole genome amplification and efficiency of gene editing was measured by droplet digital PCR (ddPCR) (FIG. 1A-1B).

A range of doses (12.5-500 μg/mL) was investigated. The mean level of editing increased with increasing NP dosage (FIG. 1A and Table 3)), which may be due to increased delivery of PNA leading to more potential binding and editing events in treated embryos. However, at all NP doses tested, embryonic gene editing was statistically significant (p<0.0001) when analyzed by a one-way ANOVA test comparing each treatment group to untreated controls (Table 3) and even the lowest tested dose of 12.5 μg/mL resulted in an average level of editing of 67% (Table 3). At the highest dose tested of 500 μg/mL, a 94% average editing of each blastocyst was achieved. 19 of the 21 blastocysts investigated at the 500 μg/mL dose exhibited very high levels of editing approaching 100% and 2 embryos exhibited editing around 50% (FIG. 1A and Table 3).

TABLE 3
a quantification of embryonic editing as analyzed by ddPCR (FIG. 1B).
At the highest tested dose of 500 μg/mL an average of 94% editing was
obtained with 19/21 embryos tested at nearly 100% editing. P-values
were calculated by one-way ANOVA. Treated and vehicle groups were
compared to untreated controls. The p-value of the blank nanoparticle
treated group was >0.9999 relative to the untreated control (no
statistically significant difference between vehicle and control groups).
The p-values of all treated groups indicate there are statistically
significant differences between treated groups and controls (p < 0.0001).

| Treatment | | | |
|---|---|---|---|
| Group | Mean % | | Number of |
| (μg/mL) | Editing | p-vaiue | Blastocysts |
| 12.5 | 67.408 | <0.0001 | 20 |
| 25 | 68.934 | <0.0001 | 20 |
| 50 | 70.272 | <0.0001 | 20 |
| 100 | 77.567 | <0.0001 | 23 |
| 200 | 80.665 | <0.0001 | 21 |
| 300 | 81.319 | <0.0001 | 20 |
| 400 | 83.842 | <0.0001 | 20 |
| 500 | 93.588 | <0.0001 | 21 |

Most treated embryos showed editing rates between 0-100%, indicating that gene editing is occurring later than the single-cell stage, since the mouse model is hemizygous and therefore has one eGFP allele at the single-cell stage.

NPs delivered to the cytoplasm may enter the nucleus during mitosis and nuclear envelope breakdown. This can result in intra-nuclear delivery of PNA at different developmental stages. Furthermore, PNA binding events may or may not lead to editing depending on kinetics and availability of donor DNA template.

Additionally, during early embryonic development, the genome is transcriptionally silenced and contains all the necessary signals for early cell divisions without any genomic transcription (Jukam et al., Dev Cell, 42(4): p. 316-332 (2017)). The embryo then undergoes the maternal-to-zygotic transition (MZT) when maternal mRNAs are degraded, and the genome then becomes transcriptionally active (Jukam et al., Dev Cell, 42(4): p. 316-332 (2017)). Prior to the MZT, genomic DNA exists in a repressed state that is incompatible with transcription (Langley et al., Development, 141(20): p. 3834-41(2014)). PNA-mediated editing acts through binding and formation of a PNA/DNA/PNA triplex that induces homologous recombination and genome editing (Ricciardi et al., Molecules, 23(3) (2018)). Since DNA methylation and tight chromatin organization contributes to transcriptional repression in the early embryo (Langley et al., Development, 141(20): p. 3834-41 (2014)), this could block or inhibit the ability of the PNA to bind and induce editing. The MZT does not occur until after the 2-cell stage in mice and after the 4-cell stage in humans (Jukam et al., Dev Cell, 42(4): p. 316-332 (2017)). This mechanism is supported by monitoring the eGFP fluorescence of the treated embryos. Embryos begin to express eGFP at measurable levels past e4.5 (at the blastocyst stage). If PNA-mediated editing does not occur until the MZT occurs when the embryo becomes transcriptionally active, PNA binding and editing would not be thought to begin until after the two-cell stage in murine embryos. PNA binding and gene editing in vitro, followed by eGFP expression to measurable levels takes less than 48 hours (Bahal et al., Nat Commun, 7: p. 13304 (2016)). Thus, if there is no interference in PNA binding and editing from the transcriptional repression of the early embryo, there should be measurable eGFP expression at or before e3.5. However, since eGFP expression is not seen until the blastocyst stage (e4.5/e5.5), genomic silencing and transcriptional repression until after the two-cell stage (e2.5) in murine embryos may inhibit PNA-binding and/or PNA-mediated gene editing. These factors together may explain why some embryos exhibit a mosaic phenotype.

### Example 2: Embryos can be Safely and Effectively Reimplanted

### Materials and Methods

Reimplantation Studies

Embryos were harvested from female donor mice according to previously discussed methods. For embryos reimplanted at the single cell stage, embryos were treated for one hour in 50 μL KSOM-AA media containing 500 μg/mL PNA/DNA NPs before transfer to surrogate moms. Embryos implanted at later developmental timepoints were cultured according to above protocols then transferred to a 50 μL droplet of KSOM-AA just prior to reimplantation. Embryos at varying ages were transferred to pseudopregnant recipient females that had been previously mated with vasectomized males. All embryo transfers were done in accordance with the protocols of the Yale Genome Editing Center. Fetal and postnatal development were observed. Genomic DNA for analysis by ddPCR and sequencing methods was isolated using the Reliaprep™ gDNA Tissue Miniprep System (Promega; Madison, Wis.).

### Results

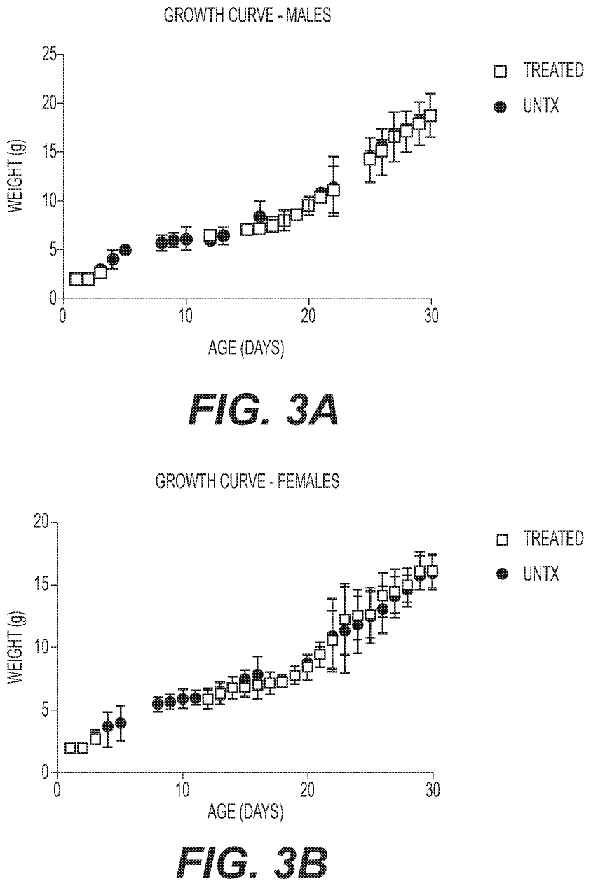

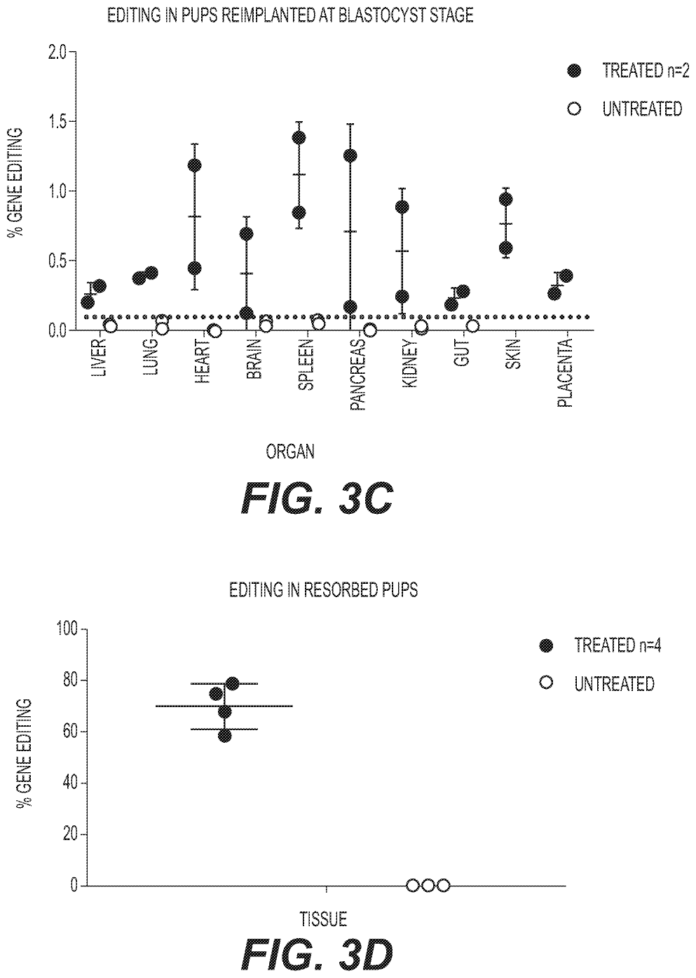

Embryos were treated for one hour with PLGA PNA/DNA NPs and then cultured for varying time-points before transfer to surrogate moms. All pregnancies resulted in fetuses that appeared healthy and exhibited normal morphology. Neonates were healthy with no visible defects and normal growth and development (FIGS. 3A and 3B).

Treated embryos reimplanted at the single-cell and two-cell stage had a development rate after reimplantation similar to that expected for untreated embryos. Reimplantation rates of untreated embryos can range from 0-75% resultant in successful implantation with an average survival rate to birth of approximately 50% (Sarvari et al., Med Biotechnol, 5(1): p. 62-5 (2013), Itoi et al., PLoS One, 7(10): p. e47512 (2012)). 64 of 139 embryos treated at either the one or two cell stage successfully implanted and developed (46%) compared to 27 of 53 of untreated single-cell reimplanted embryos (51%). Pronuclear injection of CRISPR/Cas9 results in an expected development rate after reimplantation of approximately 30% (Doe & Boroviak, Methods and Protocols, 1(1) (2018)), a significant embryo loss. PNA/DNA NP treatment may therefore indicate a gentler method for achieving site-specific embryonic gene editing.

Single and two-cell reimplanted embryos analyzed as fetuses on embryonic day e18.5 or as pups on post-natal day p30 exhibited low-levels of editing (less than 3%).

Based on the results of studies with dye loaded nanoparticles, nanoparticles are present within the cell after one hour and PNA is widespread throughout the embryo one day after treatment at the 2-cell stage. This is further indicative that PNA binding and editing is occurring later than the two-cell stage since reimplanted embryos at these stages exhibit little editing. The reimplantation process may encourage NP and/or PNA clearing from the embryo, due to the differences in the in vivo environment of the oviduct and uterus. In vivo, oviductal fluid nourishes the early embryo as it journeys to the uterus and within the uterus the embryo is bathed in endometrial secretions (Ezzati et al., J Assist Reprod Genet, 31(10): p. 1337-47 (2014)). PNA persists within the embryo in in vitro culture for at least 5 days.

However, there may be increased fluid exchange in vivo which could lead to clearing of PNA not observed in vitro. Additionally, the oviduct contains many cilia which serve to move the embryo along. Thus, if PNA is cleared in vitro, it can easily be taken back up by the stationary embryo and thus can bind within the nucleus and induce editing once again. However, in vivo, the embryo travels down the fallopian tube into the uterus, and thus if PNA is cleared it is less likely to be taken up again. Therefore, there may be clearing of NPs and/or PNA from the embryo in vivo before they are able to initiate gene editing, due to blocking of PNA binding and/or editing before the MZT occurs, which could explain the lack of editing in reimplanted single-cell and two-cell pups.

Furthermore, editing may be assisted by NPs associated with the zona pellucida or within the perivitelline space of the embryo that have not yet entered into the cell. These NPs may enter later than one hour, allowing for additional PNA delivery at later time-points, which could occur after transcriptional activation of the embryo. As the oviduct contains many ciliary cells for transport of gametes and embryos (Lyons et al., Hum Reprod Update, 12(4): p. 363-72 (2006)), these could potentially disrupt particles associated around the developing embryo, thus inhibiting their entry to the zygote.

Additionally, the protein milieu of the oviduct and uterus are considerably different than that of cellular growth media or blood (Lyons et al., Hum Reprod Update, 12(4): p. 363-72 (2006)), where PLGA NPs have been shown to be stable (Bahal et al., Nat Commun, 7: p. 13304 (2016)). Therefore, the protein corona formed by NPs in the oviduct and uterus is likely quite different than the protein corona formed by NPs introduced to blood or media. This could destabilize particles within the embryo before reimplantation occurs, since there is fluid exchange with the surrounding environment, or could destabilize NPs associated with the ZP or the perivitelline space, leading to reduced delivery of PNA/DNA editing reagents.

Experiments were designed to determine if implantations performed at later stages of embryogenesis would result in higher editing without significantly reduced viability or impaired development.

Embryos were then treated at the single-cell stage, cultured to the blastocyst stage, and then reimplanted to surrogate moms. Pups exhibited normal growth and morphology, and no gross developmental defects, again indicating that NP treatment is not cytotoxic.

However, healthy pups exhibited low levels of editing (FIG. 3C). Pups in the process of spontaneous resorption in the uterus of the surrogate mom, however, exhibited high levels of editing, with an average of 70.3% (FIG. 3D). Successful editing results in a functional eGFP gene, and so successfully edited cells will begin expressing eGFP within 48 hours. Since this is a large protein and takes energy for the cell to express, this likely gives non-edited cells a selection advantage, encouraging chimeras to proliferate unedited cells over edited cells. Furthermore, it is known that stably expressed GFP can induce proteomic changes and impair multiple cellular activities including cellular growth and contraction, (Coumans et al., Exp Cell Res, 320(1): p. 33-45 (2014)) and even induce apoptosis (Ansari et al., Stem Cell Rev, 12(5): p. 553-559 (2016)). High levels of stably expressed GFP have been shown to impair embryonic development, worsen the developmental outcomes of offspring, and to reduce the number of embryos that successfully develop to term (Bordignon et al., Biol Reprod, 68(6): p. 2013-23 (2003)). Highly edited blastocysts therefore potentially face developmental delays and an overall reduction in their developmental potential (Devgan et al., Biochem Biophys Res Commun, 313(4): p. 1030-6 (2004)). Early fetuses that are highly edited are also more likely to exhibit cardiac defects associated with eGFP over-expression (Bordignon et al., Biol Reprod, 68(6): p. 2013-23 (2003), Devgan et al., Biochem Biophys Res Commun, 313(4): p. 1030-6 (2004)). If a conceptus is developmentally delayed as exhibited by reduced growth, or has cardiac defects, maternal immune cells are more likely to induce resorption of the offspring (Flores et al., Reprod Biol Endocrinol, 12: p. 38 (2014)). This may explain the spontaneous resorption of highly edited pups seen. (FIG. 3D).

The experiments above exemplify a technique to deliver polymeric nanoparticles (NPs) loaded with peptide nucleic acids (PNAs) and donor DNA to achieve site-specific, non-enzymatic genome editing in single-cell fertilized mouse embryos. PNA/DNA NP treatment did not cause significant cytotoxicity and allowed for normal cell morphology, cellular division, and zona hatching at the correct developmental stage. Addition of dye-loaded NPs to culture medium results in NP uptake with widespread distribution across the embryo after one hour, including distribution to the nucleus of the embryo. Administration of NPs containing PNA conjugated to a TAMRA fluorophore indicates there is widespread distribution of PNA across the embryo, including the nucleus, and the PNA persists up to at least five days. PNA/DNA NP treatment resulted in 94% gene correction at a dose 500 μg/mL when measured at the blastocyst stage. Embryos reimplanted at varying stages of development also exhibit normal growth and morphology with little to no embryo loss due to treatment, making PNA/DNA NP treatment an effective and gentler alternative to CRISPR microinjections.

However, embryos reimplanted at the single-cell stage show low levels of editing. It may be that NP treatment could be administered later during embryogenesis and the same editing results could be achieved, if the PNA is unable to bind and induce editing before the MZT occurs. Furthermore, the maternal to zygotic transition occurs later in humans than in murine embryos (4-cell vs. 2-cell stage stage).

Editing may be occurring at later stages due to NPs internalized, associated with the zona pellucida, or NPs in the media which are somehow disrupted during the implantation process. Different polymers may be more stable in the fallopian tube due to formation of differing protein coronas or differing reactions to the dynamic flow and beating cilia of the fallopian tube.

In addition to human clinical implications including, for example, in vitro fertilization (IVF) for the treatment or prevention of human diseases, this PNA/DNA NP editing technique may have applications to the creation of genetic mouse models and to improving the output of livestock products, implantations performed as early during embryogenesis as possible could result in higher livestock/rodent offspring yield. The technique may be particularly effective in the treatment of human monogenetic disorders such as cystic fibrosis where both parents may be homozygous for a mutation, the treatment of autosomal dominant disorders such as Huntington's disease, or to treat recessive X-linked disorders such as Duchenne muscular dystrophy or Hemophilia A. Embryonic gene editing may also have applications in the prevention of diseases such as Alzheimer's disease, by modifying the ApoE4 risk allele, or in reduction in susceptibility to HIV infection, by editing the CCR5 receptor.

In conclusion, this approach may represent an improvement over other techniques for editing fertilized mouse embryos that involve pronuclear microinjection or electroporation of engineered nucleases, i.e. CRISPR/Cas9. These procedures are labor intensive and can result in significant embryo loss. In contrast, embryonic gene editing with PNA/DNA NPs could be a safe and relatively undemanding alternative that simply involves pipetting NPs into the embryo culture media.

### Example 3: Naked PNA and Donor DNA have an Effect, but that NPs with the Same PNAs and DNA are More Effective

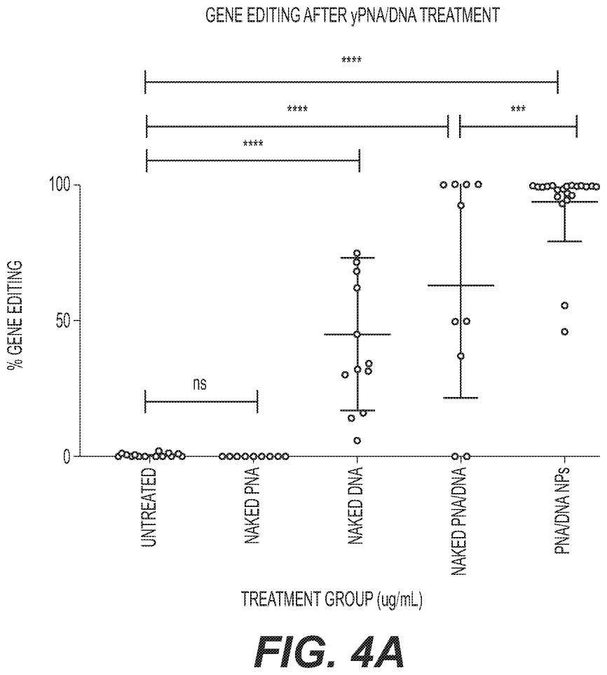

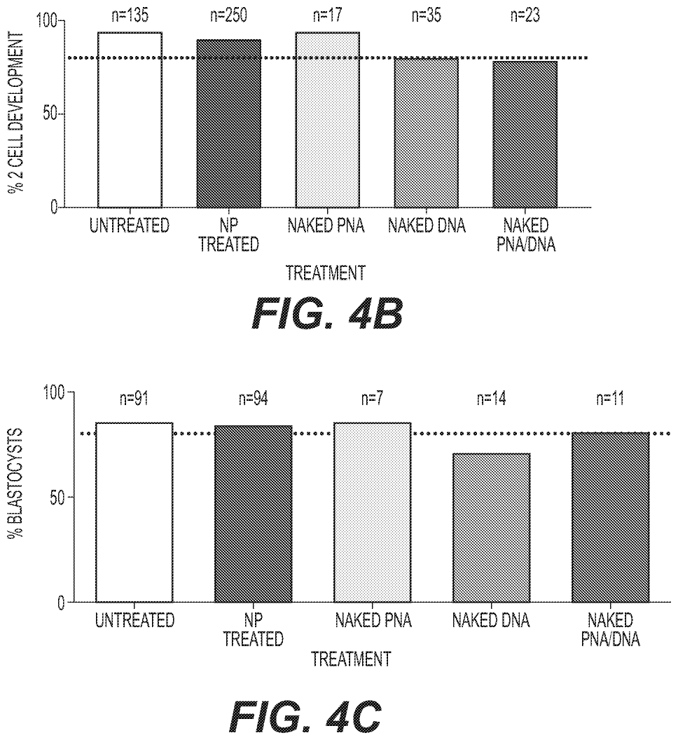

Embryos were treated at the single-cell stage with naked PNA and/or DNA oligonucleotides, which were delivered by pipetting directly onto embryo culture media. Naked oligonucleotides were given at the maximum theoretical dose of oligonucleotides encapsulated in PLGA NPs, (assuming 100% encapsulation efficiency). Embryos were analyzed by ddPCR at the blastocyst stage. Naked PNA alone does not result in any editing. Editing is significantly improved with administration of nanoparticles with PNA/DNA encapsulated over naked DNA or naked PNA and DNA alone. The results are illustrated in FIG. 4A and Table 4.

TABLE 4
Quantification of ddPCR editing data for treatment with naked
oligonucleotides. Naked PNA editing is non-significant from control
untreated editing data. Naked DNA, naked PNA/DNA, and PNA/DNA
NPs result in significant editing compared to untreated controls.
PNA/DNA NPs also result in significantly improved editing compared
to naked oligonucleotides alone (p < 0.0001 for naked DNA, and
p = 0.001 for naked PNA/DNA).

| | Mean % | | Number of |
|---|---|---|---|
| Treatment Group | Editing | p-value | Blastocysts |
| Naked PNA | 0.076 | >0.9999 | 9 |
| Naked DNA | 45.115 | <0.0001 | 13 |
| Naked PNA/DNA | 62.880 | <0.0001 | 10 |
| 500 μg/mL PLGA NPs | 93.588 | <0.0001 | 21 |

Safety is illustrated by embryo survival in FIGS. 4B and 4C. Survival of embryos to the 2-cell stage is shown FIG. 4B. Untreated embryos are expected to have greater than 80% development to the 2-cell stage. Treatment with naked oligos does not impair development to the 2-cell stage.

Survival of embryos to the blastocyst stage is shown in FIG. 4C. Untreated embryos are expected to have greater than 80% development to the blastocyst stage. Treatment with naked oligos does not appear to significantly impair development to the blastocyst stage. Some slight cytotoxicity may be caused by administration of naked DNA oligonucleotides (71.4% survival to blastocyst stage with naked DNA treatment, and 81.0% survival to blastocyst stage with naked PNA and DNA treatment, but this is more likely to be due to the small sample size, n=14 and n=11 respectively).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method comprising contacting an embryo, male gametes, female gametes, or a combination thereof in vitro with an effective amount of non-enzymatic gene editing active agent(s) to induce at least one alteration in the genome of the embryo, male gametes, female gametes, or combination thereof.

2. The method of claim 1, wherein the effective amount of non-enzymatic gene editing active agent(s) is encapsulated, entrapped, complexed to or dispersed in polymeric particles.

3. The method of claim 1, wherein the embryo is 1, 2, 4, 8, or 16 cells.

4. The method of claim 1, wherein the embryo is a zygote, morula, or blastocyst.

5. The method of claim 1, wherein the embryo, male gametes, female gametes, or a combination thereof is contacted with the particles on culture days 0-6 during or following in vitro fertilization.

6. The method of claim 5, wherein the in vitro fertilization comprises co-incubation of male and female gametes on or prior to culture day 0.

7. The method of claim 1, wherein the contacting comprises adding the particles or active agents to liquid media bathing the embryo and/or gametes.

8. The method of claim 1, wherein the embryo is a single cell embryo.

9. The method of claim 1, wherein the alteration corrects a mutation associated with a genetic disease or disorder.

10. The method of claim 9, wherein the genetic disease or disorder is a monogenic disease or disorder.

11. The method of claim 9, wherein the disease or disorder is a hemophilia, a hemoglobinopathy, cystic fibrosis, xeroderma pigmentosum, a lysosomal storage disease, Huntington's disease, Duchenne muscular dystrophy or Alzheimer's disease.

12. The method of claim 2, wherein the particles are selected from the group consisting of nanoparticles, microparticles, and mixtures thereof.

13. The method of claim 12, wherein the particles are formed of a polymer, copolymer or polymer blend selected from the group consisting of poly(lactic acid) (PLA), poly(glycolic acid) (PGA), poly(lactide-co-glycolide) (PLGA), polyesters, polyanhydrides, poly(ortho)esters, poly(butic acid), poly(valeric acid), poly(caprolactone), poly(hydroxyalkanoates), poly(lactide-co-caprolactone), poly(beta-amino) esters (PBAEs) and poly(amine-co-ester) polymers (PACE).

14. The method of claim 1, wherein the particles are nanoparticles comprising PLGA.

15. The method of claim 1, wherein the gene editing active agent(s) comprises a peptide nucleic acid (PNA) tail clamp that enhances recombination of a donor oligonucleotide in the embryonic genome relative to the donor oligonucleotide alone.

16. The method of claim 15, wherein the gene editing active agent(s) further comprises the donor oligonucleotide.

17. The method of claim 13, wherein the PNA tail clamp comprises at least one PNA oligomer.

18. The method of claim 17, wherein the at least one PNA oligomer of the PNA tail clamp is a modified PNA oligomer comprising at least one modification at a gamma position of a backbone carbon.

19. The method of claim 18, wherein the modified PNA oligomer comprises at least one miniPEG modification at a gamma position of a backbone carbon.

20. The method of claim 1, further comprising transferring the embryo into the uterus a female.

21. The method of claim 20, wherein the contacting occurs with the embryo after the maternal-to-zygotic transition (MZT), and the transferring occurs thereafter before or during the blastocyst stage.

22. The method of claim 20, wherein the contacting occurs with the embryo during the one cell stage, and the transferring occurs thereafter before or during the blastocyst stage.

23. The method of claim 1, wherein the embryo is a mammalian embryo.

24. The method of claim 23, wherein the mammal is a human.

25. The method of claim 1, wherein the embryo is an embryo of a domesticated, agricultural, or wild animal.

26. The method of claim 25, wherein the agricultural animal is a horse, cow, bull, steer, heifer, pig, sheep, rabbit, chicken, or goat.
